(19) United States
(12) Patent Application Publication
(10) Pub. No.: US 2018/0023199 A1
(43) Pub. Date: Jan. 25, 2018

(54) ELECTROCATALYTIC HYDROGEN EVOLUTION AND BIOMASS UPGRADING

(71) Applicant: Yujie Sun, North Logan, UT (US)

(72) Inventor: Yujie Sun, North Logan, UT (US)

(73) Assignee: UTAH STATE UNIVERSITY, North Logan, UT (US)

(21) Appl. No.: 15/650,884

(22) Filed: Jul. 15, 2017

Related U.S. Application Data
(60) Provisional application No. 62/364,231, filed on Jul. 19, 2016.

(51) Int. Cl. C25B 1/02; C07C 31/08; C07D 307/48; H01M 4/58; C07C 33/22

(52) U.S. Cl. CPC C25B 1/02; H01M 4/5805; H01M 4/5815; C07C 33/22; C07D 307/48; C07C 31/08

(57) ABSTRACT

Disclosed are systems for producing hydrogen gas and upgrading biomass reactants. The systems are able to couple the oxidation of the biomass reactant to hydrogen gas evolution using catalysts that include a metal component and a non-metal component. Also disclosed are methods of using the systems for producing hydrogen gas and upgrading a biomass reactant.

### CROSS-REFERENCE TO RELATED APPLICATIONS

This application claims priority to U.S. Provisional Application No. 62/364,231 filed on July 19, 2016, which is incorporated fully herein by reference.

### BACKGROUND

As a practical and environmentally friendly approach to generate clean H₂, electrocatalytic water splitting has attracted worldwide interest. However, its broad employment has been inhibited by costly and scarce catalysts and the low energy conversion efficiency mainly due to the sluggish anodic half reaction of O₂ evolution, whose product O₂ is not of significant value. Accordingly, improved systems and methods are needed for efficient and cost-effective generation of hydrogen gas.

### SUMMARY

In some aspects, disclosed are systems for producing hydrogen gas and upgrading a biomass reactant, the system comprising an anode compartment comprising an anode, and an anode solution comprising water, a first electrolyte, and an alcohol or an aldehyde derived from a lignocellulosic biomass; and a cathode compartment comprising a cathode, and a cathode solution comprising water and a second electrolyte; wherein the anode and the cathode each independently comprise a catalyst loaded onto a conductive substrate, the catalyst having a metal component and a non-metal component, wherein the metal component is selected from the group consisting of cobalt, nickel, iron, copper, manganese and a combination thereof; and the non-metal component is selected from the group consisting of phosphorous, sulfur, nitrogen, oxygen and a combination thereof.

In some aspects, disclosed are methods for producing hydrogen gas and upgrading a biomass reactant, the method comprising applying a voltage to the cathode and the anode of the systems disclosed herein, whereupon applying the voltage, the alcohol or the aldehyde derived from a lignocellulosic biomass is oxidized in the anode compartment to provide an aldehyde or a carboxylic acid biomass product and H⁺ is reduced in the cathode compartment to provide hydrogen gas.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 is an x-ray diffraction (XRD) pattern of Ni₂P nanoparticle arrays on nickel foam (Ni₂P NPA/NF) with corresponding standard patterns of Ni₂P and Ni.

FIG. 2 is a scanning electron microscopy (SEM) image of Ni₂P NPA/NF.

FIG. 3 is an SEM image of Ni₂P NPA/NF at a higher magnification than FIG. 2.

FIG. 4 includes an SEM image and corresponding elemental mapping images of Ni₂P NPA/NF.

FIG. 5 is a graph showing linear sweep voltammetry (LSV) curves of Ni₂P NPA/NF at a scan rate of about 2 mV s⁻¹ in about 1.0 M KOH with and without about 10 mM 5-hydroxymethylfrufural (HMF).

FIG. 6 is a graph showing conversion and yield (%) changes of HMF and its oxidation products during electrochemical oxidation of HMF at about 1.423 V vs RHE in about 1.0 M KOH with about 10 mM HMF.

FIG. 7 is a bar chart showing Faradaic efficiencies of Ni₂P NPA/NF for 2,5-furandicarboxylic acid (FDCA) production under three successive electrolysis cycles.

FIG. 8 is a graph showing LSV curves of Ni₂P NPA/NF at a scan rate of about 2 mV s⁻¹ in about 1.0 M KOH with and without about 10 mM HMF.

FIG. 9 is a graph showing Tafel plots corresponding to FIG. 8 of Ni₂P NPA/NF at a scan rate of about 2 mV s⁻¹ in about 1.0 M KOH with and without about 10 mM HMF.

FIG. 10 is a graph showing the chronopotentiometric curve of Ni₂P NPA/NF at about 10 mA cm⁻² in about 1.0 M KOH containing about 10 mM HMF. The inset shows the expanded zigzag chronopotentiometric curve due to growth and release of H₂ bubbles on the catalyst surface.

FIG. 11 is a graph showing LSV curves for a Ni₂P NPA/NF catalyst couple in about 1.0 M KOH with and without about 10 mM HMF.

FIG. 12 is a bar chart showing a comparison of the overpotentials to achieve benchmark current densities (about 10, about 20, and about 50 mA cm⁻²) for a Ni₂P NPA/NF catalyst couple in about 1.0 M KOH with and without about 10 mM HMF.

FIG. 13 is a graph showing the gas chromatography (GC)-measured H₂ quantity compared with theoretically calculated H₂ quantity assuming a 100% Faradaic efficiency for the H₂ evolution catalyzed by a Ni₂P NPA/NF catalyst couple in about 1.0 M KOH solution with about 10 mM HMF.

FIG. 14 is a bar chart showing Faradaic efficiencies of Ni₂P NPA/NF catalyst couple for simultaneous H₂ and FDCA generation in about 1.0 M KOH solution with about 10 mM HMF for three successive electrolysis cycles.

FIG. 15 is an SEM image of pristine Ni foam (NF) at low magnification.

FIG. 16 is an SEM image of pristine Ni foam (NF) at high magnification.

FIG. 17 is a high-resolution XPS spectra of Ni 2_{3/2} for fresh Ni₂P NPA/NF samples.

FIG. 18 is a high-resolution XPS spectra of P 2p for fresh Ni₂P NPA/NF samples.

FIG. 19 is a graph showing LSV curves of Ni₂p NPF/NF (red) and Ni foam (blue) for HMF (about 10 mM) oxidation in about 1.0 M KOH (scan rate about 2 mV s⁻¹). The blank control of Ni foam was water oxidation is also included for comparison.

FIG. 20 is a graph showing a comparison of charge vs time curves for Ni₂P NPA/NF (red) and Ni foam (blue) during controlled potential electrolysis (applied potential about 1.423 V vs RHE) in about 1.0 M KOH with about 10 mM HMF.

FIG. 21 is a graph showing a comparison of current vs time curves for Ni₂P NPA/NF (red) and Ni foam (blue) during controlled potential electrolysis (applied potential about 1.423 V vs RHE) in about 1.0 M KOH with about 10 mM HMF.

FIG. 22 is a graph showing controlled potential electrolysis of Ni₂P NPA/NF in about 1.0 M KOH containing about 10 mM HMF at a potential of about 1.423 V vs RHE.

FIG. 23 is a series of chromatograms for HMF oxidation catalyzed by Ni₂P NPA/NF at about 1.423 V vs RHE over passed charge.

FIG. 24 is a series of chromatograms for HMF oxidation catalyzed by Ni₂P NPA/NF at about 1.423 V vs RHE over passed charge.

FIG. 25 is a series of chromatograms for HMF oxidation catalyzed by Ni₂P NPA/NF at about 1.423 V vs RHE over passed charge. The inset is a picture of the electrolyte solution at about 0 C and about 58.99 C showing the color change from yellow to transparent.

FIG. 26 is a series of SEM images of post-HMF Ni₂P NPA/NF sample.

FIG. 27 is an SEM image and the corresponding elemental mapping images of post-HMF Ni₂P NPA/NF sample.

FIG. 28 is a graph showing the XRD patterns of fresh (black), post-HER with HMF (red), and post-HMF (blue) Ni₂P NPA/NF samples.

FIG. 29 is a graph showing high-resolution Ni 2p_{3/2} spectra for fresh, post-HER with HMF, and post-HMF Ni2P NPA/NF samples.

FIG. 30 is a graph showing high-resolution P 2p XPS spectra for fresh, post-HER with HMF, and post-HMF Ni₂P NPA/NF samples.

FIG. 31 is an SEM image of post-HER with HMF Ni₂P NPA/NF sample.

FIG. 32 is an SEM image of post-HER with HMF Ni₂P NPA/NF sample.

FIG. 33 is an SEM image and the corresponding elemental mapping images of post-HER with HMF Ni₂P NPA/NF sample.

FIG. 34 is a graph showing linear sweep voltammograms of Ni₃S₂/NF in about 1.0 M KOH with (red) and without (black) about 10 mM HMF.

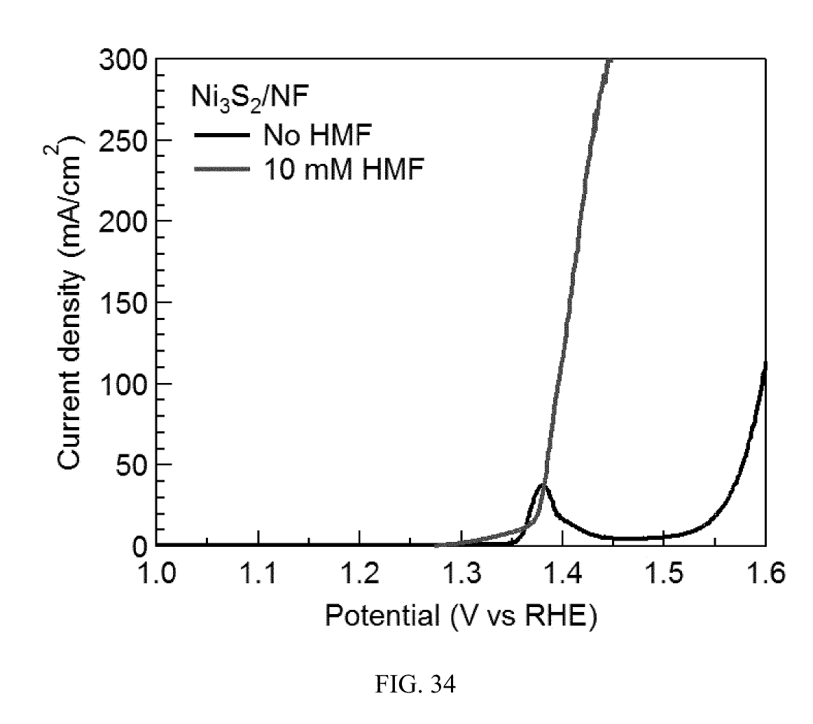

FIG. 35 is a graph showing linear sweep voltammograms of Ni/NF in about 1.0 M KOH with (red) and without (black) about 10 mM HMF.

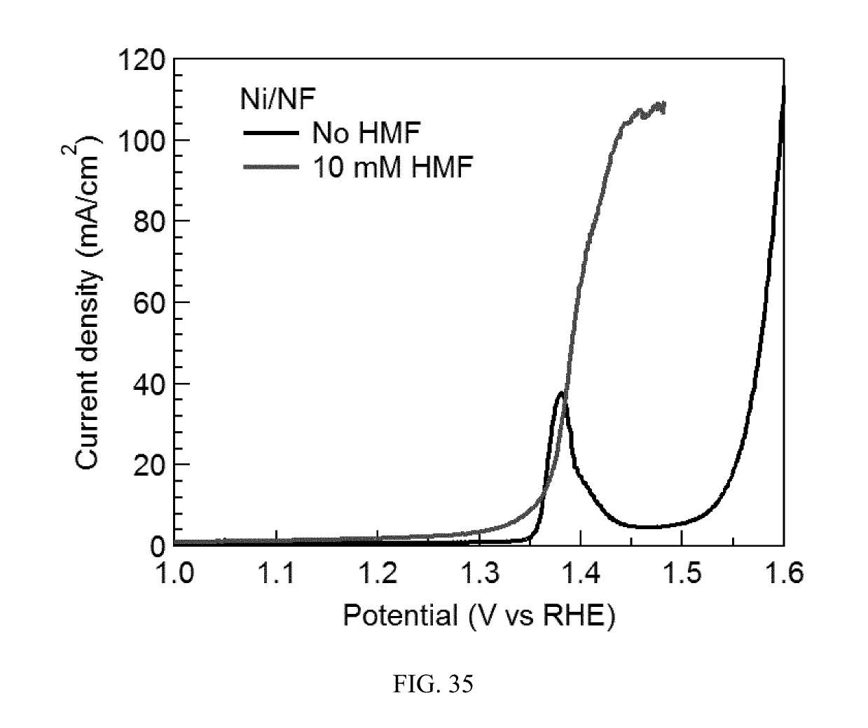

FIG. 36 is a graph showing linear sweep voltammograms of Ni₂P/NF in about 1.0 M KOH with (red) and without (black) about 10 mM HMF.

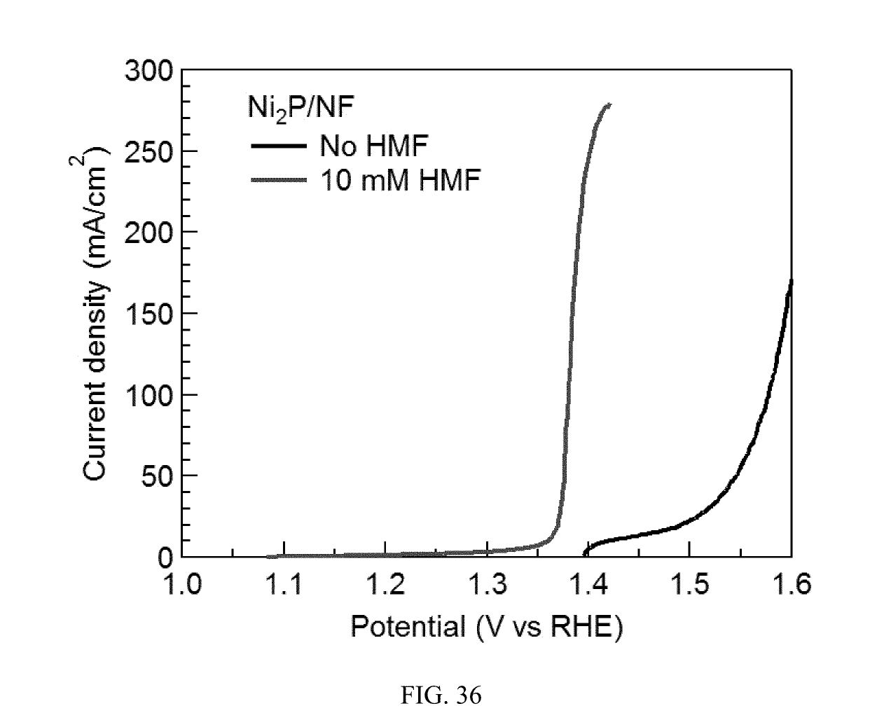

FIG. 37 is a graph showing linear sweep voltammograms of Co—P/CF in about 1.0 M KOH with (red) and without (black) about 50 mM HMF.

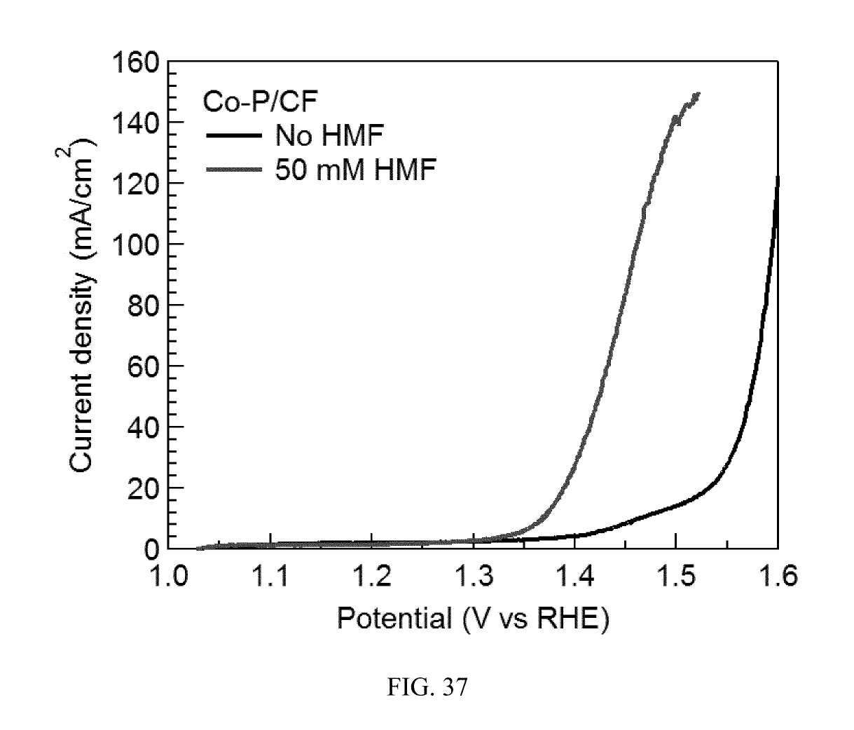

FIG. 38 is a graph showing linear sweep voltammograms of Ni₂P/NF in about 1.0 M KOH with (red) and without (black) about 30 mM furfural.

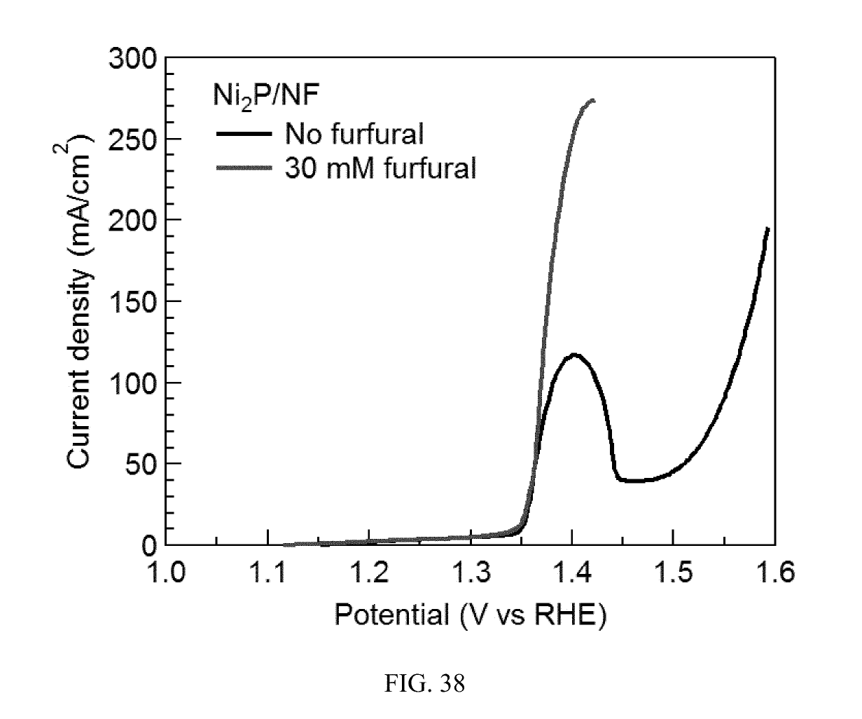

FIG. 39 is a graph showing linear sweep voltammograms of Ni₃N/NF in about 1.0 M KOH with (red) and without (black) about 30 mM furfural.

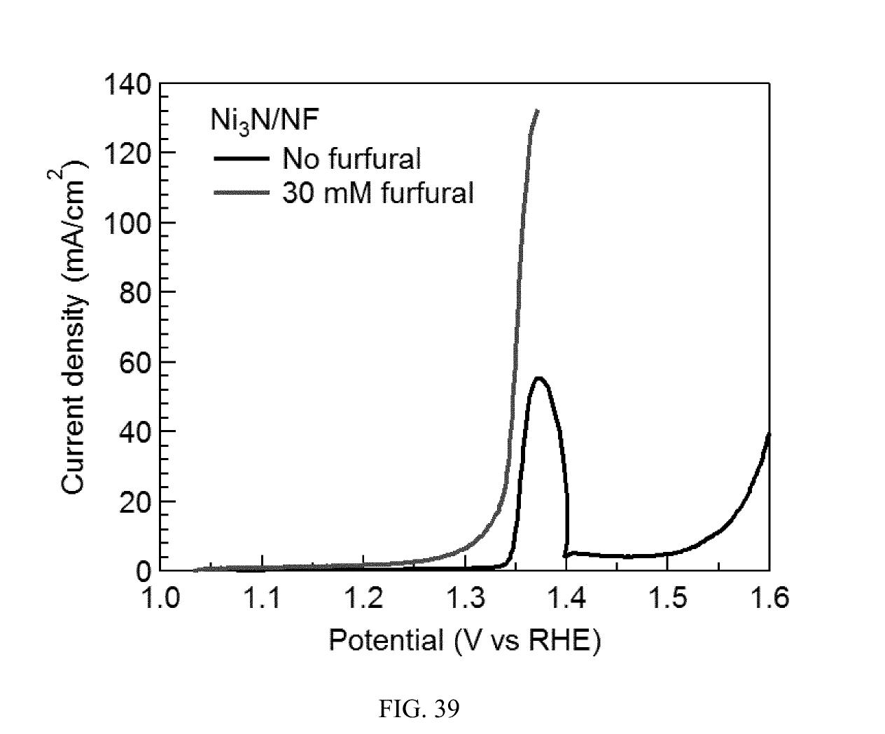

FIG. 40 is a series of SEM images of (a) NF and (b-c) Ni/NF at different magnifications.

FIG. 41 is a series of SEM images of Ni₂P/Ni/NF at different magnifications.

FIG. 42 is an SEM image and the elemental mapping images of Ni₂P/Ni/NF showing the distributions of Ni and P.

FIG. 43 is a graph showing cathodic scans of cyclic voltammograms of Ni₂P/Ni/NF with (red) and without (black) about 30 mM furfural at a scan rate of about 2 mV/s in about 1.0 M KOH.

FIG. 44 is a bar chart showing a comparison of the current density of Ni₂P/NUNF and NF at about 1.40, about 1.42, and about 1.44 V vs RHE in about 1.0 M KOH with about 30 mM furfural.

FIG. 45 is a graph showing yield curves of 2-furoic acid at about 1.423 V vs RHE in about 1.0 M KOH with about 30 mM furfural.

FIG. 46 is a graph showing the Ni 2p region for Ni₂P/NUNF after (red) and before (black) electrocatalytic furfural oxidation.

FIG. 47 is a graph showing LSV curves of Ni₂P/NUNF couple with (red) and without (black) about 30 mM furfural in about 1.0 M KOH.

FIG. 48 is a bar chart showing a comparison of the current densities of Ni₂P/NUNF couple at about 1.50, about 1.55, and about 1.60 V vs RHE in about 1.0 M KOH with (red) and without (black) about 30 mM furfural.

FIG. 49 is a bar chart showing the yield of 2-furoic acid catalyzed by the same Ni₂P/NUNF catalyst couple used in FIG. 48 in about 1.0 M KOH for five successive electrolysis cycles with about 30 mM furfural for each cycle.

FIG. 50 is a graph showing the GC-measured H₂ quantity (red) compared with the calculated H₂ quantity (black) based on passed charge during electrocatalysis with a Ni₂P/NUNF catalyst couple in about 1.0 M KOH solution.

FIG. 51 is a series of schematics showing chemical reactions for integrated H₂ production and alcohol oxidation in alkaline media.

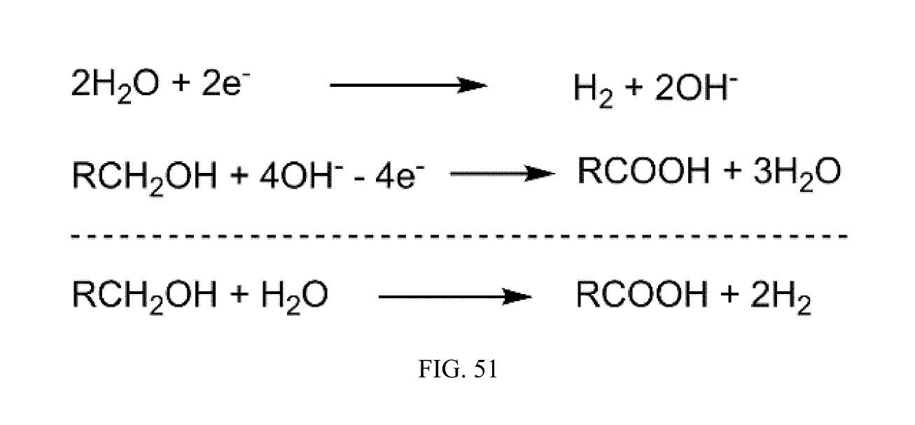

FIG. 52 is a graph showing the XRD pattern of porous Ni microspheres on a nickel foam (hp-Ni) and the standard pattern of metallic Ni.

FIG. 53 is an SEM image of hp-Ni.

FIG. 54 is an SEM image of hp-Ni at higher magnifications than in FIG. 53.

FIG. 55 is a graph showing linear sweep voltammograms of hp-Ni at about 2 mV s⁻¹ in about 1.0 M KOH containing about 0 and about 10 mM benzyl alcohol (BA).

FIG. 56 is a graph showing conversion or yield (%) dependence of BA and its oxidation products on passed charges during electrochemical oxidation at about 1.423 V vs. RHE in about 10 mL, about 1.0 M KOH with about 10 mM BA (Ph-CHO: benzyl aldehyde; Ph-COOH: benzoic acid).

FIG. 57 is a bar chart showing Faradaic efficiencies of benzoic acid production for controlled potential electrolysis with hp-Ni in about 10 mL, about 1.0 M KOH with about 10 mM BA at about 1.423 V vs. RHE under five successive cycles.

FIG. 58 is a graph showing LSV curves of hp-Ni at about 2 mV s⁻¹ in about 1.0 M KOH with about 10 mM BA, 4-methylbenzyl alcohol (MBA), or 4-nitrobenzyl alcohol (NBA).

FIG. 59 is a schematic showing (a) the pathway of benzyl alcohol (BA) oxidation to benzoic acid and (b) the chemical structures of MBA and NBA.

FIG. 60 is a pair of SEM images of post-BA hp-Ni at different magnifications.

FIG. 61 is an SEM image and the corresponding elemental mapping of post-BA hp-Ni.

FIG. 62 is a pair of graphs showing high-resolution XPS spectra of (d) Ni 2p_{3/2} and (e) O 1s is for fresh and post-BA hp-Ni electrocatalysts.

FIG. 63 is a schematic and a graph showing oxidation of ethanol to value-added acids and the corresponding linear sweep voltammograms of hp-Ni at about 2 mV s⁻¹ in about 1.0 M KOH in the presence (red) and absence (black) of about 10 mM alcohol substrates.

FIG. 64 is a schematic and a graph showing oxidation of HMF to value-added acids and the corresponding linear sweep voltammograms of hp-Ni at about 2 mV s⁻¹ in about 1.0 M KOH in the presence (red) and absence (black) of about 10 mM alcohol substrates.

FIG. 65 is a graph showing high-performance liquid chromatography (HPLC) traces of electrocatalytic HMF oxidation catalyzed by hp-Ni at about 1.423 V vs. RHE in about 10 mL, about 1.0 M KOH with about 10 mM HMF.

FIG. 66 is a graph showing conversion of HMF or yield of the oxidation products during electrolysis.

FIG. 67 is a bar chart showing Faradaic efficiencies of FDCA production using the same hp-Ni catalyst for five successive cycles.

FIG. 68 is a graph showing linear sweep voltammograms of hp-Ni couple in about 1.0 M KOH containing about 0 and about 10 mM BA.

FIG. 69 is a bar chart showing the comparison of overpotential at various current densities utilizing the hp-Ni couple in about 1.0 M KOH containing about 0 and about 10 mM BA.

FIG. 70 is a graph showing experimental H₂ quantity compared with the theoretical quantity during HER catalyzed by hp-Ni couple.

FIG. 71 is an SEM image of Co—P/CF before electrocatalytic HMF oxidation. The inset shows the SEM image of an as-prepared Co—P film at a higher magnification.

FIG. 72 is an SEM image of Co—P/CF after electrocatalytic HMF oxidation.

FIG. 73 is a graph showing Co 2p regions for Co—P/CF before (top) and after (bottom) electrocatalytic HMF oxidation.

FIG. 74 is a graph showing P 2p regions for Co—P/CF before (top) and after (bottom) electrocatalytic HMF oxidation.

FIG. 75 is a graph showing linear sweep voltammograms of Co—P/CF in about 1.0 M KOH in the absence (black) and presence of about 30 mM HMF (red) at a scan rate of about 2 mV/s.

FIG. 76 is a graph showing the conversion of HMF and yields of those oxidation products over passed charge during a chronoamperometry experiment conducted at about 1.423 V vs RHE in about 1.0 M KOH containing about 50 mM HMF.

FIG. 77 is a graph showing linear sweep voltammograms of a Co—P film obtained in about 1.0 M KOH with the addition of about 30 mM DFF (purple), about 30 mM HMFCA (green), about 30 mM FFCA (blue), or no organic substrates (black).

FIG. 78 is a graph showing accumulated charge over time of a chronoamperometry experiment at about 1.423 V vs RHE for Co—P in about 1.0 M KOH containing about 10 mM HMF and with the continuous addition of about 10 mM HMF twice. Inset shows the corresponding current change over time.

FIG. 79 is a graph showing linear sweep voltammograms of a Co—P/Co—P catalyst couple obtained in the absence (black) and presence (red) of about 50 mM HMF and about 1.0 M KOH (scan rate about 2 mV/s).

FIG. 80 is a graph showing GC-measured H₂ quantity (red) compared with theoretically calculated H₂ quantity (black) assuming a 100% Faradaic efficiency for the H₂ evolution of a Co—P/Co—P catalyst couple in about 1.0 M KOH solution with about 50 mM HMF.

FIG. 81 is an illustration of a traditional electrolyzer.

FIG. 82 is an illustration of an ECPB-mediated electrolyzer.

FIG. 83 is an illustration of an electrolyzer embodiment disclosed herein.

FIG. 84 is an SEM image of as-prepared Ni₃S₂/NF.

FIG. 85 is an SEM image of as-prepared Ni₃S₂/NF.

FIG. 86 is an SEM image of as-prepared Ni₃S₂/NF.

FIG. 87 is an SEM image and the element mapping images of as-prepared Ni₃S₂/NF showing the element distributions of Ni and S.

FIG. 88 is a series of schematics and graphs showing oxidation of selected organics to value-added products and the corresponding LSV curves of Ni₃S₂/NF at a scan rate of about 2 mV s⁻¹ in about 1.0 M KOH with and without about 10 mM organic substrates (BA: benzyl alcohol; FFA: furfuryl alcohol; FF: furfural; HMF: 5-hydroxymethylfurfural).

FIG. 89 is a schematic showing two possible pathways of HMF oxidation to FDCA.

FIG. 90 is a graph showing HPLC traces of electrolysis of HMF oxidation catalyzed by Ni₃S₂/NF at about 1.423 V vs RHE in about 10 mL, about 1.0 M KOH with about 10 mM HMF.

FIG. 91 is a graph showing conversion and yield (%) changes of HMF and its oxidation products during electrochemical oxidation of HMF.

FIG. 92 is a bar chart showing Faradic efficiencies of Ni₃S₂/NF for FDCA production under five successive cycles.

FIG. 93 is a series of SEM images of post-HMF Ni₃S₂/NF at different magnifications.

FIG. 94 is an SEM image and the element mapping images of post-HMF Ni₃S₂/NF showing the element distributions of Ni and S, plus a large amount of O.

FIG. 95 is a graph showing LSV curves of Ni₃S₂/NF for H₂ evolution at a scan rate of about 2 mV s⁻¹ in about 1.0 M KOH with and without about 10 mM HMF.

FIG. 96 is a graph showing Tafel plots corresponding with FIG. 95 of Ni₃S₂/NF for H₂ evolution at a scan rate of about 2 mV s⁻¹ in about 1.0 M KOH with and without about 10 mM HMF.

FIG. 97 is a graph showing a chronopotentiometric curve of Ni₃S₂/NF for H₂ evolution at about −10 mA cm⁻² in about 1.0 M KOH containing about 10 mM HMF. The inset shows the expanded zigzag chronopotentiometric curve due to the growth and release of H₂ bubbles on the catalyst surface.

FIG. 98 is an SEM image of post-HER with HMF Ni₃S₂/NF.

FIG. 99 is an SEM image of post-HER with HMF Ni₃S₂/NF.

FIG. 100 is an SEM image of post-HER with HMF Ni₃S₂/NF.

FIG. 101 is an SEM image and the element mapping images of post-HER with HMF Ni₃S₂/NF showing the element distributions of Ni and S.

FIG. 102 is a graph showing LSV curves for Ni₃S₂/NF couple in about 1.0 M KOH with and without about 10 mM HMF.

FIG. 103 is a bar chart showing a comparison of the overpotentials to achieve benchmark current densities for Ni₃S₂/NF couple in about 1.0 M KOH with and without about 10 mM HMF.

FIG. 104 is a graph showing GC-measured H₂ quantity compared with theoretically calculated H₂ quantity assuming a 100% Faradaic efficiency for the H₂ evolution catalyzed by a Ni₃S₂/NF catalyst couple in about 1.0 M KOH solution with about 10 mM HMF.

### DETAILED DESCRIPTION

Disclosed herein are systems and methods that can address the various needs and problems regarding the generation of hydrogen gas by providing an efficient strategy to replace O₂ evolution reaction (OER) with a thermodynamically more favorable reaction, such as the oxidation of alcohols or aldehydes derived from lignocellulosic biomass to a corresponding aldehyde or carboxylic acid biomass product (e.g. upgrading biomass) through the use of bifunctional catalysts. Alcohols or aldehydes derived from a lignocellulosic biomass, such as 5-hydroxymethylfurfural (RMF), primarily arise as dehydration intermediates of raw biomass, and when oxidized to its corresponding aldehyde or carboxylic acid biomass product (e.g., HMF oxidized to 2,5-furandicarboxylic acid (FDCA)), the oxidized product may be used in a number of industrial applications. For example, FDCA may be a substitute of terephthalic acid to produce polyamides, polyesters, and polyurethanes.

The disclosed systems using bifunctional catalysts can couple the OER reaction of the alcohols or aldehyde derived from lignocellulosic biomass with the H₂ evolution reaction (HER) for hydrogen gas evolution. By coupling these two reactions using the disclosed catalysts, the disclosed systems surprisingly require significantly less voltage (e.g., at least less than 200 mV) compared to pure water splitting systems to achieve similar current densities (e.g., 50 mA/cm²), as well as exhibiting robust stability and nearly unity Faradaic efficiencies for both H₂ evolution and biomass upgrading. Such a low-cost and energy-efficient strategy is potentially a new platform for sustainable energy conversion technologies.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of and “consisting essentially of,” the embodiments or elements presented herein, whether explicitly set forth or not.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrases “at least one of A, B, . . . and N” or “at least one of A, B, . . . N, or combinations thereof' are defined in the broadest sense to mean one or more elements selected from the group comprising A, B, . . . and N, that is to say, any combination of one or more of the elements A, B, . . . or N including any one element alone or in combination with one or more of the other elements which may also include, in combination, additional elements not listed.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “alcohol or aldehyde derived from a lignocellulosic biomass” as used herein refers to an alcohol (e.g., a compound having at least one —OH group) or an aldehyde (e.g., a compound having at least one —C(O)H group) that has been derived from a lignocellulosic biomass, and can also be referred to as a biomass reactant.

Biomass refers to biological material from living, or recently living organisms, and in particular lignocellulosic biomass refers to plant biomass, and can include cellulose, hemicelluose, and/or lignin. Alcohol and aldehyde compound reactants that are derived from lignocellulosic biomass refers to lignocellulosic biomass that has been broken down to C₅ and C₆ sugars and then the alcohols or aldehydes compound reactants are derived from these C₅ and C₆ sugars through biomass pretreatment processes like pyrolysis, hydrolysis, etc.

Such techniques of breaking down lignocellulosic biomass and the products that arise are further described in “Lignocellulosic biomass: a sustainable platform for the production of bio-based chemicals and polymers” (Polym. Chem., 2015, 6, 4497-4559), which is incorporated herein by reference in its entirety. Examples of alcohols and aldehydes derived from lignocellulosic biomass include, but are not limited to, 5-hydroxymethylfurfural, 3-hydroxypropionic acid, glycerol, sorbitol, xylitol, lactic acid, ethanol, butanol, benzyl alcohol, furfural, arabinitol, xylose, methanol, and cinnamaldehyde.

The term “aldehyde biomass product,” as used herein, refers to the aldehyde product (e.g., a compound having at least one —C(O)H group) that arises from the oxidation of an alcohol derived from a lignocellulosic biomass. The term “carboxylic acid biomass product,” as used herein, refers to the carboxylic acid product (e.g., a compound having at least one —C(O)OH group) that arises from the oxidation of an alcohol or an aldehyde derived from a lignocellulosic biomass. An aldehyde biomass product or carboxylic acid biomass product also may be referred to herein simply as a “biomass product” and/or an “upgraded biomass product.” An alcohol derived from a lignocellulosic biomass, when oxidized using the disclosed systems and methods, may be converted to its corresponding aldehyde biomass product or its corresponding carboxylic acid biomass product. An aldehyde derived from a lignocellulosic biomass (which may include an aldehyde biomass product formed from oxidation of an alcohol derived from a lignocellulosic biomass) may be oxidized to its corresponding carboxylic acid biomass product. Examples of aldehyde or carboxylic acid biomass products include, but are not limited to, FDCA, acetic acid, benzoic acid, and furoic acid.

### 2. SYSTEMS FOR HYDROGEN EVOLUTION AND BIOMASS UPGRADING

Disclosed herein are systems that can produce hydrogen gas while also upgrading biomass reactant. The system may include an anode compartment and a cathode compartment.

The anode compartment includes an anode and an anode solution. The cathode compartment includes a cathode and a cathode solution. The anode compartment and the cathode compartment can be electrically connected as to form an electrochemical cell, where oxidation reactions may take place in the anode compartment and reduction reactions may take place in the cathode compartment. For example, the oxidation half reaction can take place in the anode compartment, while the reduction half reaction can take place in the cathode compartment. Or in other words, the oxidation of the alcohol or the aldehyde derived from lignocellulosic biomass can take place in the anode compartment, thereby upgrading the biomass reactant to the aldehyde or carboxylic acid biomass product. Accordingly, the reduction of H⁻ can take place in the cathode compartment, thereby producing hydrogen gas. Accordingly, the system may be configured to have a voltage applied to it, and in in some embodiments, the anode and the cathode are configured to have a voltage applied to it.

A. Anode Solution

The anode solution may include water, a first electrolyte, and an alcohol or an aldehyde derived from a lignocellulosic biomass. The first electrolyte may include potassium hydroxide, sodium hydroxide, sodium perchlorate, borate buffer, phosphate buffer, or a combination thereof. The first electrolyte may be present at a concentration of from about 0.1 M to about 5 M, such as from about 0.5 M to about 4 M or from about 1 M to about 3 M. In some embodiments, the first electrolyte is present at a concentration of greater than or equal to 0.1 M, greater than or equal to 0.2 M, greater than or equal to 0.3 M, greater than or equal to 0.4 M, or greater than or equal to 0.5 M. In some embodiments, the first electrolyte may be present at a concentration of less than or equal to 5 M, less than or equal to 4.5 M, less than or equal to 4 M, less than or equal to 3.5 M, or less than or equal to 3 M.

The alcohol or the aldehyde derived from a lignocellulosic biomass may include, but is not limited to 5-hydroxymethylfurfural (HMF), 3-hydroxypropionic acid, glycerol, sorbitol, xylitol, lactic acid, ethanol, butanol, benzyl alcohol, furfural, arabinitol, xylose, methanol, cinnamaldehyde, or combinations thereof. In some embodiments, the alcohol or the aldehyde derived from a lignocellulosic biomass may include HMF, ethanol, benzyl alcohol, furfural or combinations thereof.

The alcohol or the aldehyde derived from a lignocellulosic biomass may be included at varying concentrations in the anode solution. For example, the alcohol or the aldehyde derived from a lignocellulosic biomass may be present at a concentration of from about 1 mM to about 100 mM, such as from about 5 mM to about 75 mM or from about 1 mM to about 50 mM. In some embodiments, the alcohol or the aldehyde derived from a lignocellulosic biomass may be present at a concentration of greater than or equal to 1 mM, greater than or equal to 2 mM, greater than or equal to 3 mM, greater than or equal to 4 mM, or greater than or equal to 5 mM. In some embodiments, the alcohol or the aldehyde derived from a lignocellulosic biomass may be present at a concentration of less than or equal to 100 mM, less than or equal to 90 mM, less than or equal to 80 mM, less than or equal to 70 mM, or less than or equal to 60 mM.

B. Cathode Solution

The cathode solution may include water and a second electrolyte. Generally, the description regarding the first electrolyte can be applied to the second electrolyte. For the purposes of brevity, this description will not be repeated here. In some embodiments, the second electrolyte is the same electrolyte as the first electrolyte.

C. Catalyst

The anode and the cathode may each independently include a catalyst loaded onto a conductive substrate. The catalyst may serve to catalyze the redox half-reactions taking place in the anode and cathode compartments. The catalyst may be electrodeposited onto the conductive substrate. In some embodiments, the catalyst may be annealed onto the conductive substrate. In some embodiments the catalyst may be bifunctional, and may be used in both the anode and the cathode. In other words, the anode and the cathode may include the same catalyst and conductive substrate.

The catalyst may have a metal component and a non-metal component. Examples of the metal component include, but are not limited to, cobalt, nickel, iron, copper, manganese and combinations thereof. Examples of the non-metal component include, but are not limited to, phosphorous, sulfur, nitrogen, oxygen and combinations thereof. The metal component may be doped with the non-metal component. Examples of the conductive substrate include, but are not limited to, copper, nickel, stainless steel, glassy carbon, nickel foam, stainless steel foam, titanium, fluorine-doped tin oxide, indium-doped tin oxide, and combinations thereof.

In some embodiments, the catalyst may be selected from the group consisting of a phosphorus-doped metal, a sulfur-doped metal, and a nitrogen-doped metal. In some embodiments, the catalyst may be selected from the group consisting of cobalt phosphide, nickel phosphide, cobalt sulfide, nickel sulfide, nickel nitride, cobalt oxide, nickel oxide, and a combination thereof.

In some embodiments, the catalyst does not include any noble metals. In some embodiments, the conductive substrate does not include any noble metals. Accordingly, in some embodiments, the anode and cathode each independently do not include any noble metals.

The metal and non-metal components may be included in the catalyst at varying amounts. For example, the non-metal component and the metal component may be included at a molar ratio of from about 1:9 to about 3:2 (non-metal:metal).

The catalyst and conductive substrate may include catalytic nanoparticles (or microparticles) associated with the conductive substrate. For example, the catalyst may be Ni₂P nanoparticles associated with a conductive substrate that may be nickel foam.

The catalyst and conductive substrate may include a catalyst coating on the conductive substrate. In some embodiments, the catalyst and conductive substrate may be CoP coated onto a metal foam, such as copper foam. In some embodiments, the catalyst and conductive substrate may be Ni₂P coated on nickel foam. Other catalysts coated on nickel foam may include, but are not limited to, Ni₃S₂ and Ni₃N with sulfur or ammonia as the nonmetal element source.

In some embodiments, the anode and cathode chambers may be separated by a separator. The separator may be a porous separator. In some embodiments, the term separator is synonymous with membrane. Separators may be classified as permeable, semi-permeable, or non-permeable. The degree of permeability is dependent on the size of pores in the separator, the character (e.g., charge, hydrophobicity) of the pores, and the character of the electrolyte or electrolyte component which is to be transported across the separator. A porous separator is considered permeable to all electrolyte components, though the degree of permeability may differ for different component species of the electrolyte (e.g., based on size). A semi-permeable separator typically is selectively permeable to certain materials (e.g., small cations, small anions, H₂O) while being substantially non-permeable to other materials (e.g., large molecules, neutral species, a type of redox active material). In some embodiments, the separator is a non-porous separator permeable to ions.

The separator may be ion permeable. In some embodiments, the separator is selectively permeable to permit the flux of cations with low resistance, and may be termed “cation permeable” or “cation conductive”. In some embodiments, the separator is selectively permeable to permit the flux of anions with low resistance, and may be termed “anion permeable” or “anion conductive”. Accordingly, the separator may be cation permeable or anion permeable. An ion selective separator may comprise functional groups of opposite charge to the permitted ion, such that the charge of the functional group repels ions of like charge. In some embodiments, the separator is a cation exchange membrane. In some embodiments, the separator is an anion exchange membrane. In some embodiments, the separator is functionalized with ammonium, SO₃H, OH, COOH or a combination thereof.

The separator may have a thickness of ≦200 microns, ≦100 microns, ≦50 microns, or ≦25 microns. In some embodiments, the separator has a thickness of ≦10 microns, ≧15 microns, ≧25 microns or ≧50 microns. In some embodiments, the separator has a thickness of from about 10 microns to about 200 microns, such as from about 10 microns to about 100 microns or from about 25 microns to about 100 microns.

The system may include a third electrode, such as a reference electrode. In some embodiments, the reference electrode may be Ag/AgCl.

### 3. METHODS OF PRODUCING HYDROGEN GAS AND UPGRADING BIOMASS

Also disclosed herein are methods of producing hydrogen gas and upgrading a biomass reactant. The method may use any of the systems for hydrogen evolution and biomass upgrading as described above. In particular, the method may include applying a voltage to the cathode and the anode of any system as described above. When an electrical input, such as an applied voltage, is applied to the anode and the cathode of the system, the alcohol or the aldehyde derived from lignocellulosic biomass may be oxidized in the anode compartment to provide an aldehyde or a carboxylic acid biomass product and H⁺ may be reduced in the cathode compartment to provide hydrogen gas.

As mentioned above, by coupling HER with OER associated with the disclosed biomass upgrading may allow for a relatively low application of voltage to the system, while still providing a useful current density. For example, the applied voltage may be from about 0.1 V to about 3 V, such as from about 0.2 V to about 2.5 V or from about 0.5 V to about 1.8 V. In some embodiments, the applied voltage is less than or equal to 3 V, less than or equal to 2.5 V, less than or equal to 2 V, less than or equal to 1.8 V, or less than or equal to 1.5 V.

In addition, the applied voltages listed above may provide an effective and efficient current density. For example, the applied voltage can provide a current density of from about 5 mA/cm² to about 200 mA/cm², such as from about 10 mA/cm² to about 150 mA/cm² or from about 10 mA/cm² to about 100 mA/cm². In some embodiments, the applied voltage may provide a current density of greater than or equal to 5 mA/cm², greater than or equal to 10 mA/cm², greater than or equal to 25 mA/cm², greater than or equal to 50 mA/cm², or greater than or equal to 100 mA/cm². In some embodiments, the applied voltage may provide a current density of less than or equal to 200 mA/cm², less than or equal to 175 mA/cm², less than or equal to 150 mA/cm², less than or equal to 125 mA/cm², or less than or equal to 100 mA/cm².

The disclosed method may provide hydrogen gas and the upgraded biomass product at enhanced efficiencies. For example, the method may provide hydrogen gas at a Faradaic efficiency of greater than or equal to 95%, greater than or equal to 96%, greater than or equal to 97%, greater than or equal to 98%, or greater than or equal to 99%. In some embodiments, the method provides hydrogen gas at about 100% Faradaic efficiency.

In addition, the method may provide the aldehyde or the carboxylic acid biomass product at a Faradaic efficiency of greater than or equal to 95%, greater than or equal to 96%, greater than or equal to 97%, greater than or equal to 98%, or greater than or equal to 99%. In some embodiments, the method provides the aldehyde or the carboxylic acid biomass product at about 100% Faradaic efficiency.

The method include the anode and cathode solutions each independently at a pH from about 5 to about 10, such as from about 5 to about 9 or from about 6 to about 9. For example, the method may include the anode and cathode solutions each independently at a pH of about 5, about, 5.5, about 6, about 6.5, about 7, about 7.5, about 8, about 8.5, about 9, about 9.5, or about 10. In some embodiments, the method may include the anode and cathode solutions each independently at a pH of greater than 5, greater than 6, greater than 7, or greater than 8. In some embodiments, the method may include the anode and cathode solutions each independently at a pH of less than 10, less than 9.5, or less than 9.

The method may include the anode and the cathode solutions each independently at a temperature of from about 10° C. to about 40° C., such as from about 15° C. to about 35° C. or from about 15° C. to about 30° C. In some embodiments, the method may include the anode and cathode solutions each independently at a temperature of greater than 10° C., greater than 15° C., greater than 20° C., or greater than 25° C. In some embodiments, the method may include the anode and cathode solutions each independently at a temperature of less than 40° C., less than 35° C., less than 30° C., or less than 25° C.

The methods may include numerous types of alcohols or aldehydes derived from lignocellulosic biomass, such as (but not limited to) 5-hydroxymethylfurfural (HMF), 3-hydroxypropionic acid, glycerol, sorbitol, xylitol, lactic acid, ethanol, butanol, benzyl alcohol, furfural, arabinitol, xylose, methanol, cinnamaldehyde, or combinations thereof. As described above, these biomass reactants can be oxidized to provide an aldehyde or a carboxylic acid biomass product. In some exemplary embodiments, the alcohol or the aldehyde derived from a lignocellulosic biomass and the aldehyde or the carboxylic acid biomass product may be selected from one or a combination of the following: the alcohol or the aldehyde derived from a lignocellulosic biomass may be HMF and the aldehyde or the carboxylic acid biomass product may be 2,5-furandicarboxylic acid (FDCA); the alcohol or the aldehyde derived from lignocellulosic biomass may be ethanol and the aldehyde or the carboxylic acid biomass product may be acetic acid; the alcohol or the aldehyde derived from lignocellulosic biomass may be benzyl alcohol and the aldehyde or the carboxylic acid biomass product may be benzoic acid; the alcohol or the aldehyde derived from lignocellulosic biomass may be furfural and the aldehyde or the carboxylic acid biomass product may be furoic acid.

The production of hydrogen gas (in the cathode compartment) and the aldehyde or the carboxylic acid biomass product (in the anode compartment) can be used in a number of different applications, e.g., fuel cell, ammonia synthesis, hydrogenation in petroleum refining, polymers, drug and fine chemical synthesis. Accordingly, the disclosed methods may also include steps to separate the hydrogen gas from the cathode compartment and the aldehyde or the carboxylic acid biomass product from the anode compartment.

### 4. EXAMPLES

### Example 1

### Simultaneous H₂ Generation and Biomass Upgrading in Water via an Efficient Noble-Metal-Free Bifunctional Electrocatalyst

H₂, when generated from water splitting with renewable energy input, is a green energy carrier. Unfortunately, the sluggish kinetics of the two half-reactions of water splitting, H₂ and O₂ evolution reactions (HER and OER), requires high overpotential to achieve appreciable catalytic current density, resulting in relatively low energy conversion efficiency. In spite of certain catalyst known in the art, OER is still the bottle neck of overall water splitting and demands much higher overpotential to match the rate of HER. In addition, the product of OER, O₂, is not of significant value and the potential mixing of H₂ and O₂ in the headspace of an electrolyzer poses safety concerns, requiring costly gas separation steps. Therefore, replacing OER with thermodynamically more favorable biomass oxidation reactions would not only generate value-added products at both electrodes (H₂ and upgraded bioproducts) but also increase the energy conversion efficiency of an electrolyzer.

Biorefinery, referring to conversion of biomass into fuels and chemicals, is a complementary alternative to petroleum refining, in part because biomass consists of contemporary carbon and its utilization will not alter the current ecosystem. Among many biomass-derived intermediates, 5-hydroxymethylfrufural (HMF) can be used as a versatile precursor for the production of fine chemicals, plastics, pharmaceuticals, and liquid fuels. For instance, one of its oxidation products, 2,5-furandicarboxylic acid (FDCA, Scheme 1), can serve as a monomer to produce polyamides, polyesters, and polyurethanes, being a replacement of terephthalic acid. Most previous catalytic systems were conducted under high-pressure O₂ or air at elevated temperatures and catalyzed by precious metals, such as Au, Pd, Pt or their alloys. In this regard, electrocatalytic oxidation offers an alternative greener strategy as the conversion will be driven by electricity and no chemical oxidants are necessary.

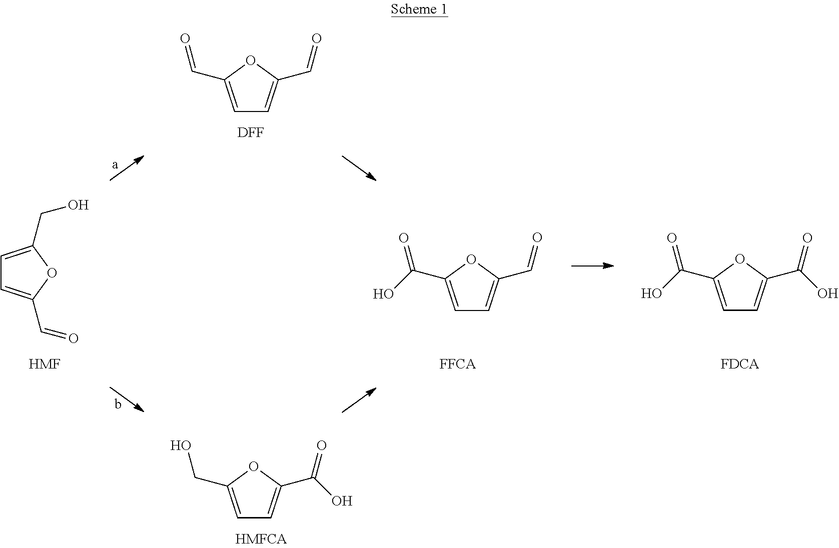

In this Example, a bifunctional electrocatalyst of 3D Ni₂P nanoparticle arrays on nickel foam (Ni₂P NPA/NF), which can couple HMF oxidation and H₂ evolution in alkaline media, is described. In this exemplary embodiment, Ni₂P NPA/NF was prepared by a scalable and lost-cost method, phosphidation of commercial nickel foam. Owing to the catalytic performance of Ni₂P NPA/NF for HMF oxidation relative to OER, a two-electrode electrolyzer employing the Ni₂P NPA/NF catalyst couple on both cathode and anode was able to produce high current density (e.g., 50 mA cm⁻²) with a voltage at least 200 mV less than that of pure water splitting electrolysis. In addition, nearly unity Faradaic efficiencies were achieved for both H₂ (100%) and FDCA (98%) generation, together with robust stability. The low-cost composite and preparation method of Ni₂P NPA/NF, as well as its markedly improved performance for H₂ and FDCA production render the disclosed catalytic systems appealing for sustainable energy conversion technologies, among other applications.

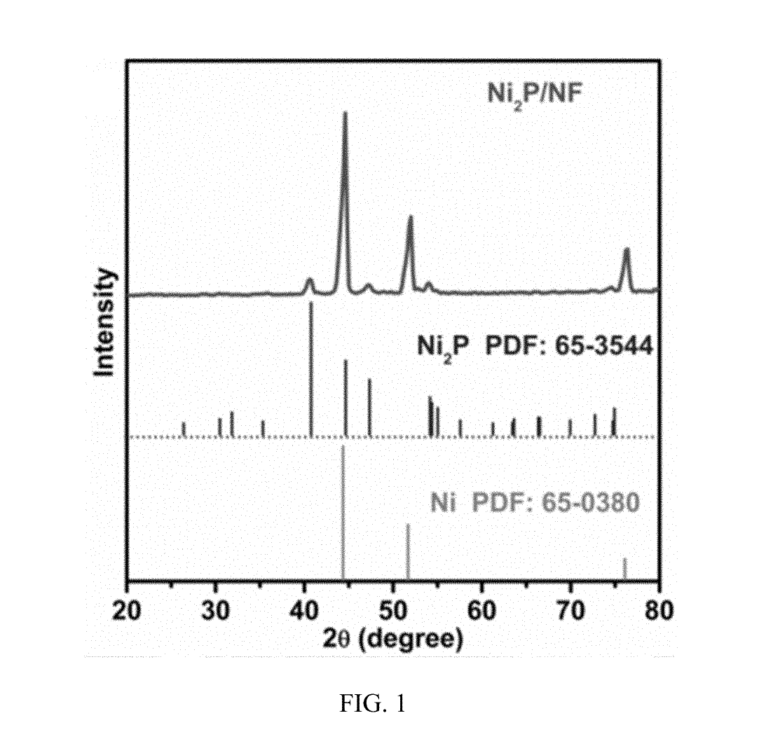

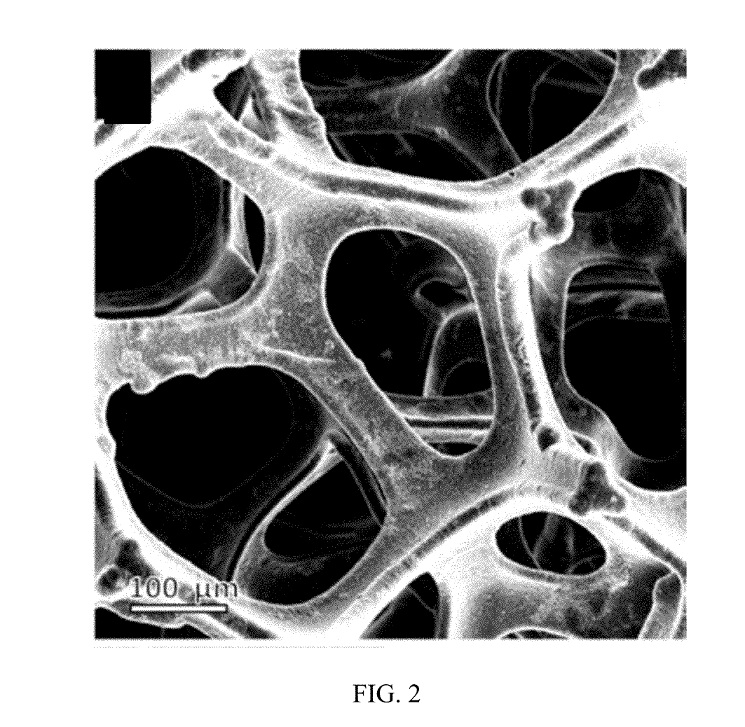

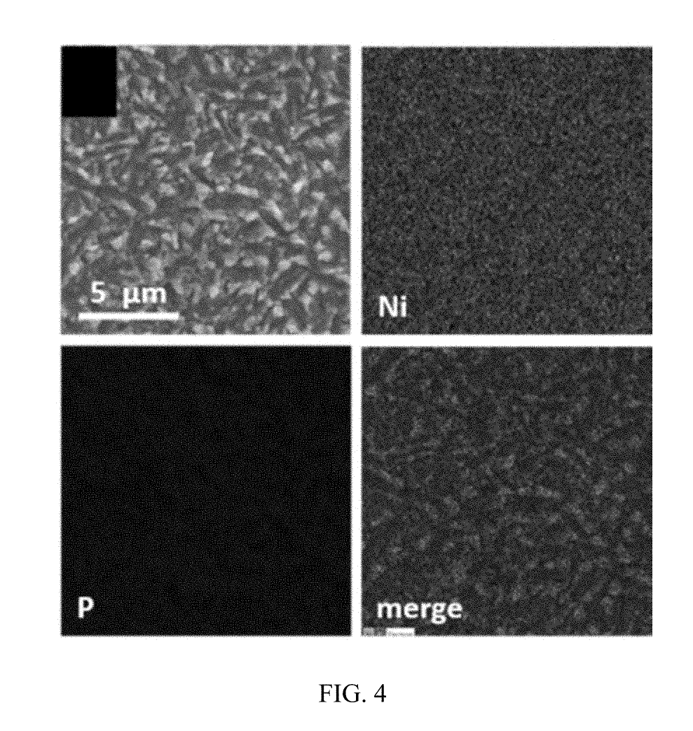

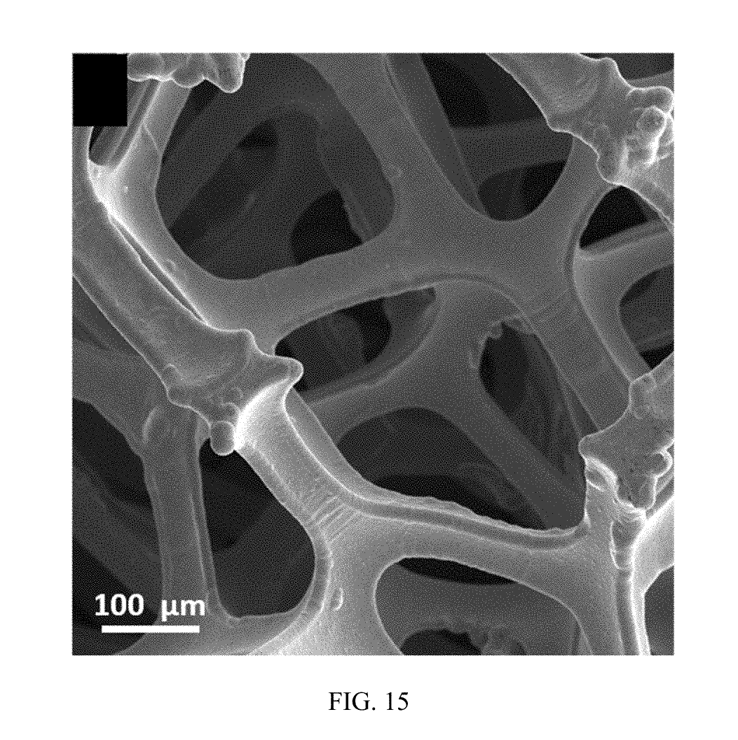

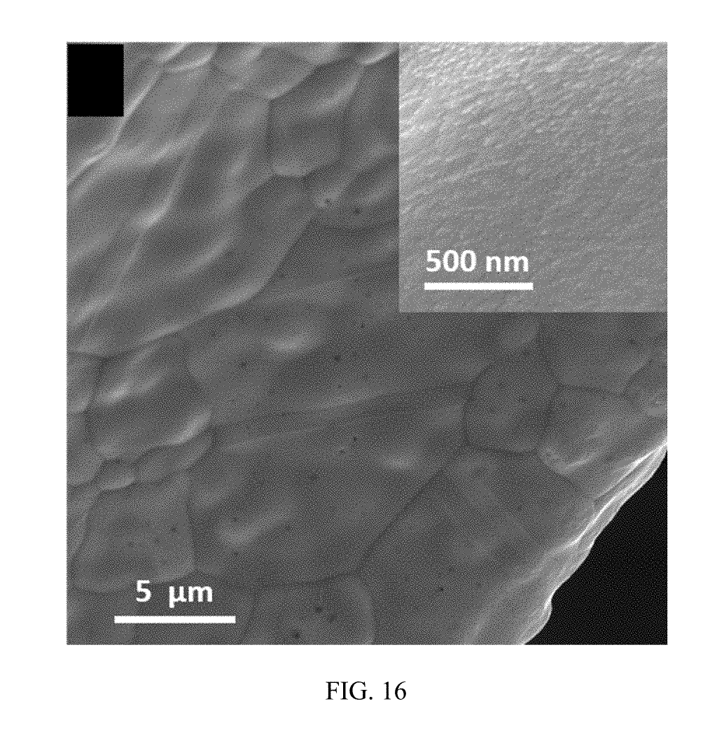

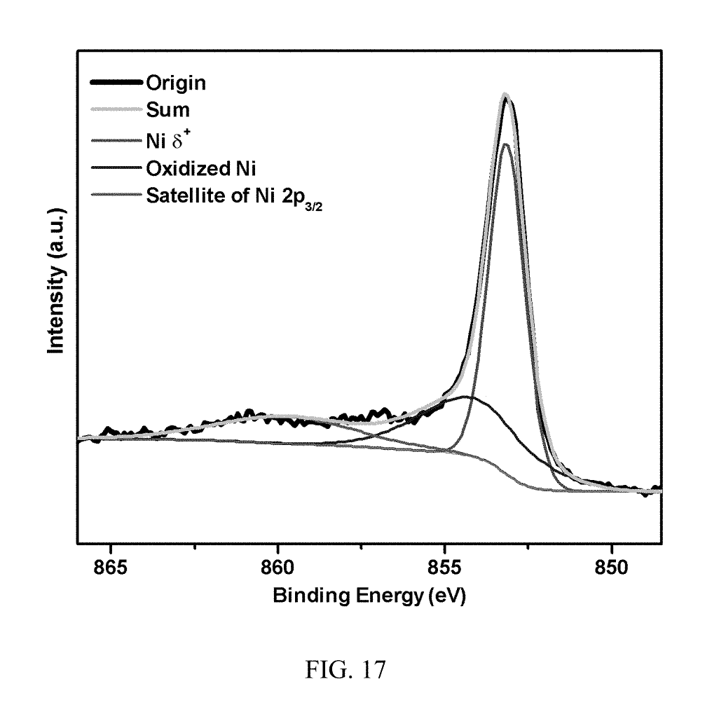

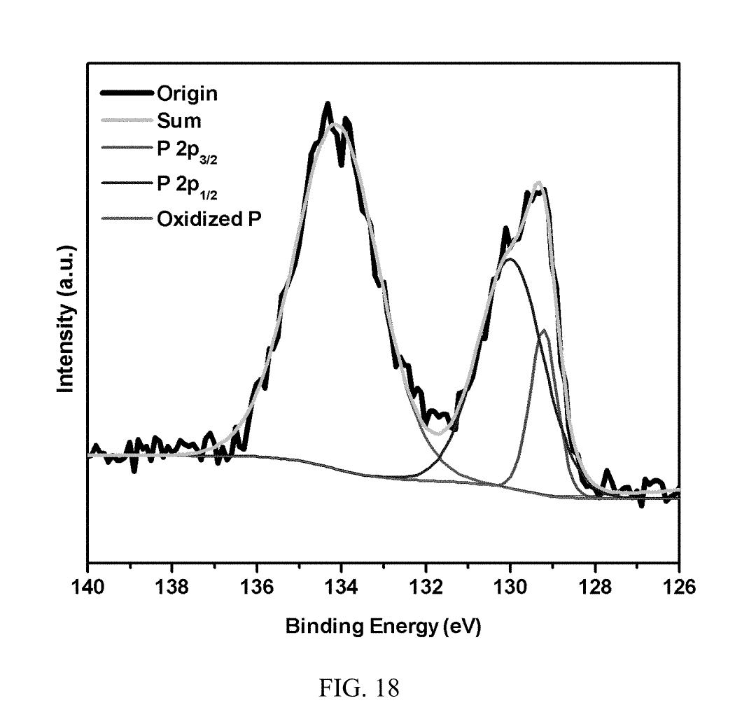

The 3D bifunctional Ni₂P NPA/NF electrocatalyst was prepared by a facile phosphidation of commercially available nickel foam as described below. X-ray diffraction (XRD) pattern (FIG. 1) confirmed the partial transformation of metallic Ni to Ni₂P (PDF: 65-3544). Scanning electron microscopy (SEM) imaging (FIG. 2) indicated that Ni₂P NPA/NF can maintain the 3D hierarchically macroporous framework of the pristine nickel foam (FIG. 15). While, the high-magnification SEM image of Ni₂P NPA/NF revealed a rough and porous morphology composed of numerous Ni₂P nanoparticles, in sharp contrast to a smooth surface of the original nickel foam (FIG. 16). FIG. 4 shows corresponding elemental mapping images of Ni and P in Ni₂P NPA/NF and indicates that both Ni and P may be uniformly distributed throughout the whole sample, in agreement with the successful chemical conversion of metallic Ni to Ni₂P via the low temperature phosphidation. X-ray photoelectron spectroscopy (XPS) analysis corroborated the presence of Ni and P in Ni₂P NPA/NF (FIGS. 17 and 18), in line with elemental mapping results (FIG. 4). The high-resolution Ni 2p_{3/2} spectrum was fitted by three sub-peaks at binding energies of about 853.1, 854.4, and about 860.4 eV (FIG. 17), which is assigned to Ni^{δ+} in Ni₂P, oxidized Ni species, and the Ni 2p_{3/2} satellite peak of Ni₂P, respectively. Similarly, the high-resolution P 2p XPS spectrum (FIG. 18) could be deconvoluted into three sub-peaks at about 129.3, 130.0, and 134.3 eV, which correspond to P 2p_{3/2}, P 2_{1/2}, and oxidized P species (possibly arising from superficial oxidation due to exposure in air), respectively. The Ni 2p peak at about 853.1 eV was positively shifted compared to that of metallic Ni (about 852.5 eV) and the P 2p peak at 129.3 eV was negatively shifted relative to that of elemental P (about 130.2 eV). These binding energy shifts imply that Ni in Ni₂P NPA/NF has a partial positive charge (δ⁺) while P has a partial negative charge (ED, indicative of charge transfer from Ni to P.

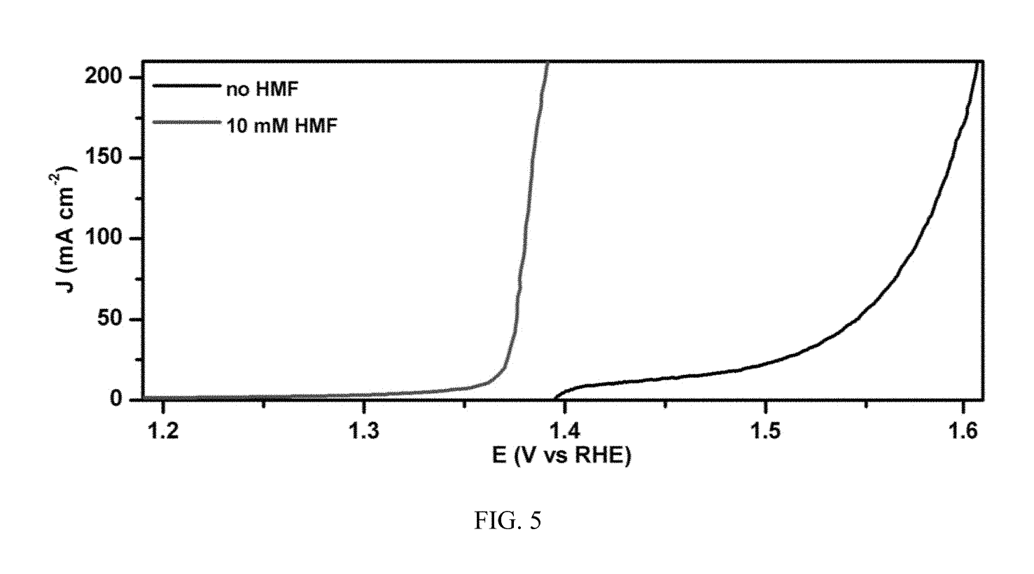

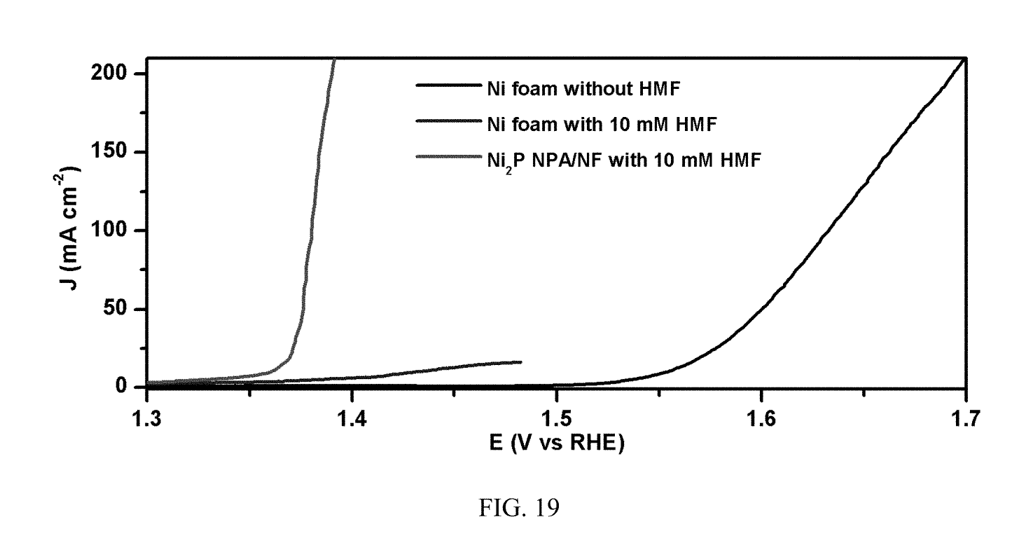

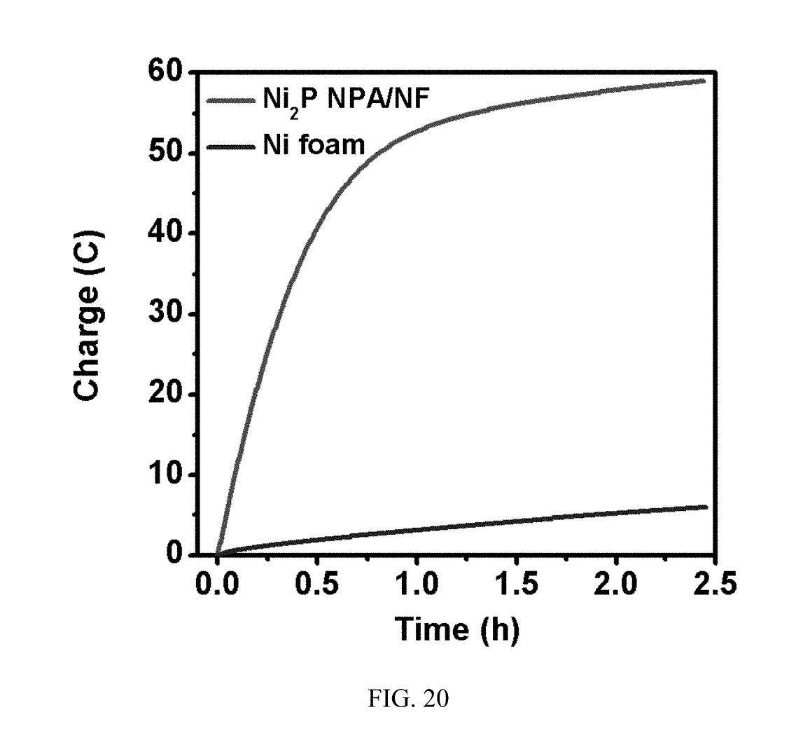

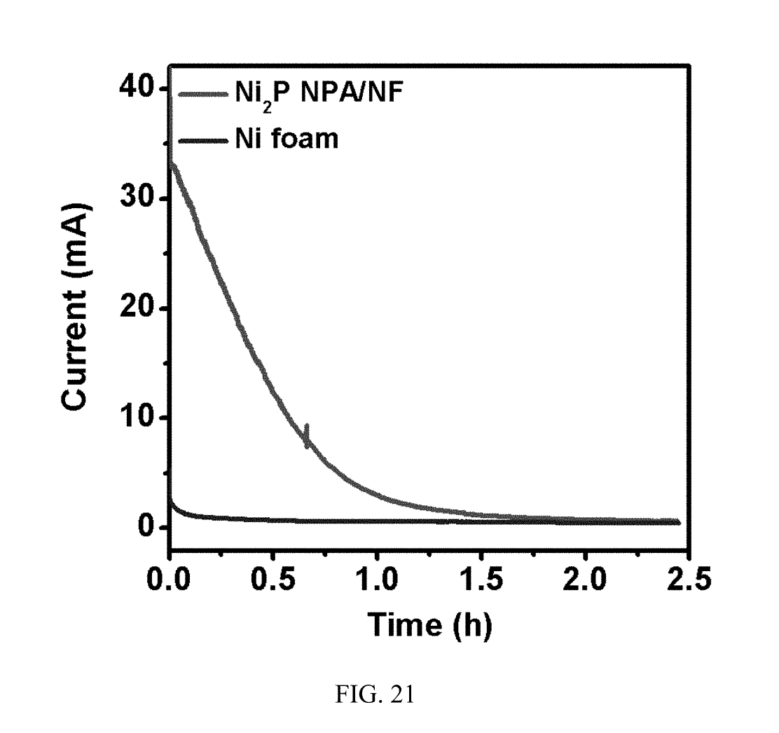

For the electrocatalytic oxidation of HMF in an aqueous electrolyte (about 1.0 M KOH), OER may be the major competing reaction. Therefore, the electrochemical HMF oxidation and OER catalyzed by Ni₂P NPA/NF were first compared via linear sweep voltammetry (LSV) in FIG. 5. In the absence of HMF, Ni₂P NPA/NF exhibited a catalytic onset potential of about 1.50 V vs RHE (reversible hydrogen electrode) and high catalytic current density beyond about 1.60 V vs RHE, implying an excellent OER activity. Upon the addition of about 10 mM HMF, the onset potential shifted to about 1.35 V vs RHE, and a rapid current density rise could be observed within about 1.40 V vs RHE, which indicates that the oxidation of HMF was significantly easier than OER catalyzed by Ni₂P NPA/NF. In this Example, pristine nickel foam showed inferior performance for both OER and HMF oxidation (FIGS. 19-21), highlighting the important role of Ni₂P on nickel foam in this case.

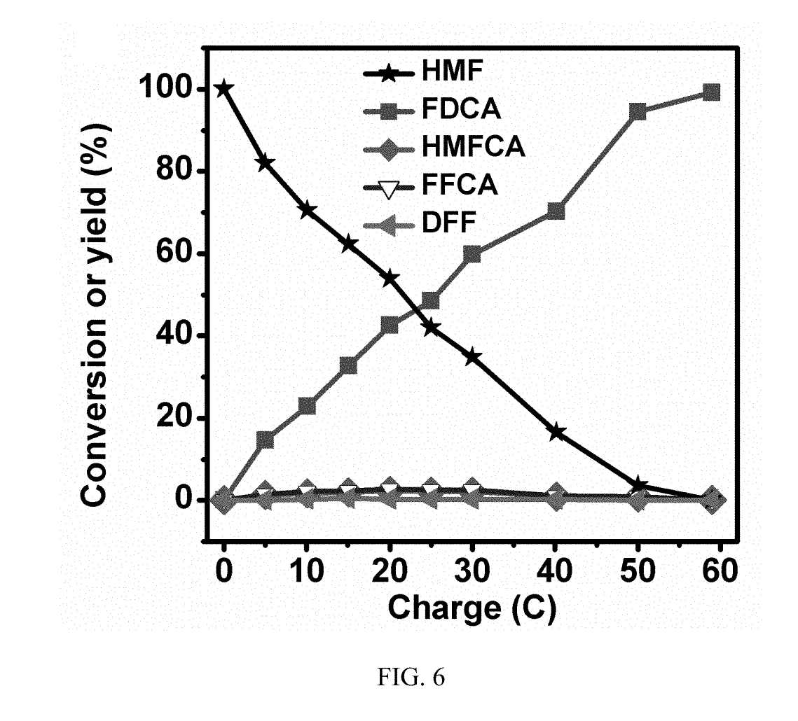

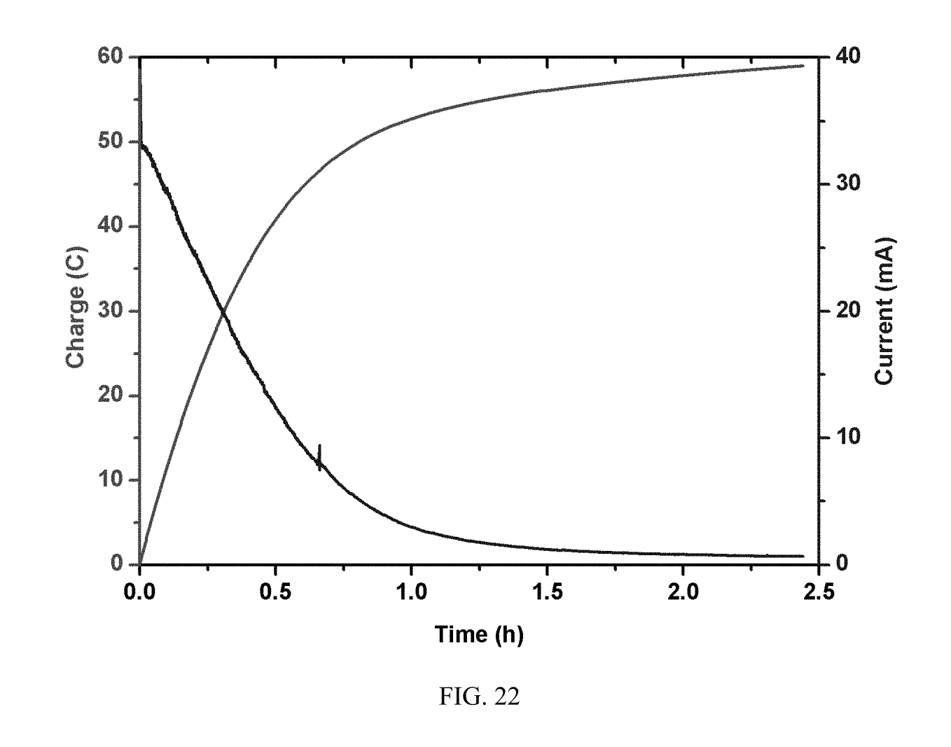

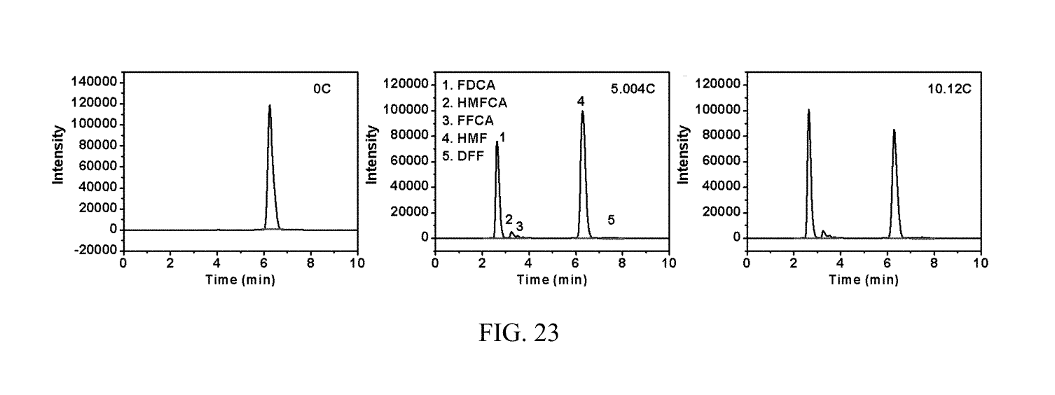

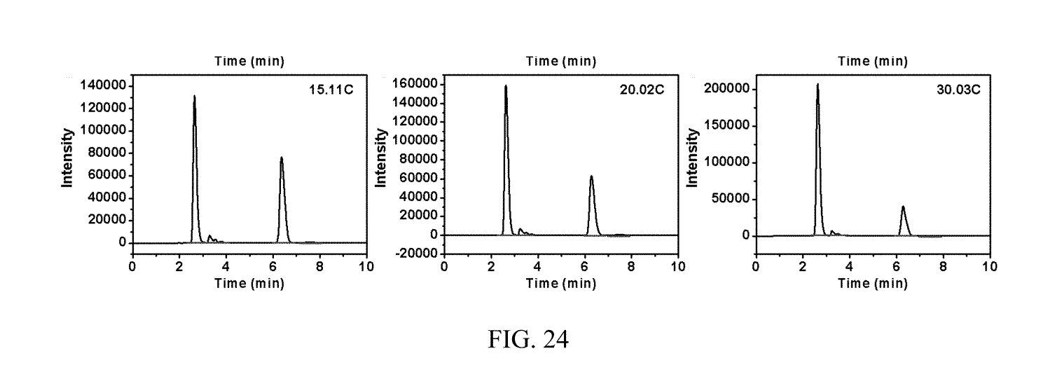

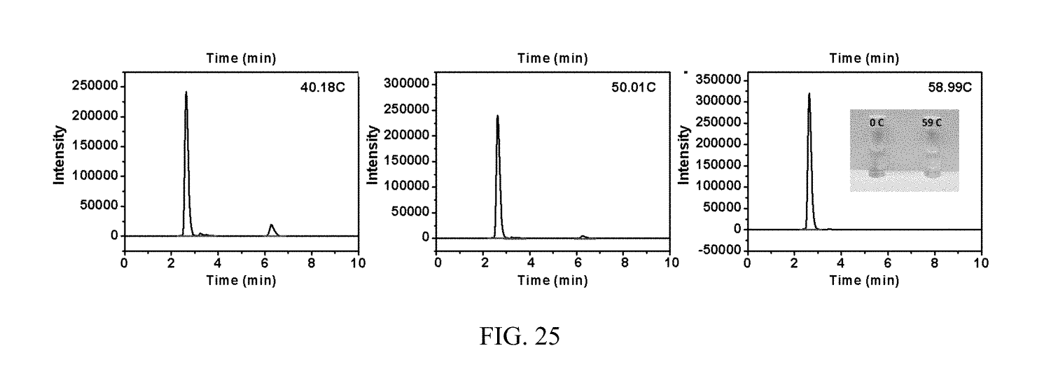

In order to identify and quantify the oxidation products as well as calculate the corresponding Faradaic efficiencies, Ni₂P NPA/NF-catalyzed HMF oxidation was performed by applying a constant potential of about 1.423 V vs RHE and passing charge of about 59 C (FIG. 22). According to calculation, about 59 C was corresponding to complete HMF oxidation to FDCA if an about 100% Faradaic efficiency could be achieved. As shown in FIG. 5, no appreciable water oxidation could occur at about 1.423 V, hence there may be a high Faradaic efficiency for HMF oxidation. High-performance liquid chromatography (HPLC) was used to monitor the concentration changes of HMF and its oxidation products during electrolysis (see below for details), and the resulting chromatograms (FIGS. 23-25) show the concentration decrease and rise of HMF and FDCA, respectively, over time, suggesting a conversion of HMF into FDCA (FIG. 6). After passing charge of about 59 C, the peak of HMF completely disappeared and the color of the electrolyte solution changed from pale yellow to colorless (FIG. 25 inset). The concentration changes of HMF, its oxidation intermediates, and FDCA during the electrolysis are plotted in FIG. 6, resulting in near unity Faradaic efficiencies for both HMF conversion and FDCA production.

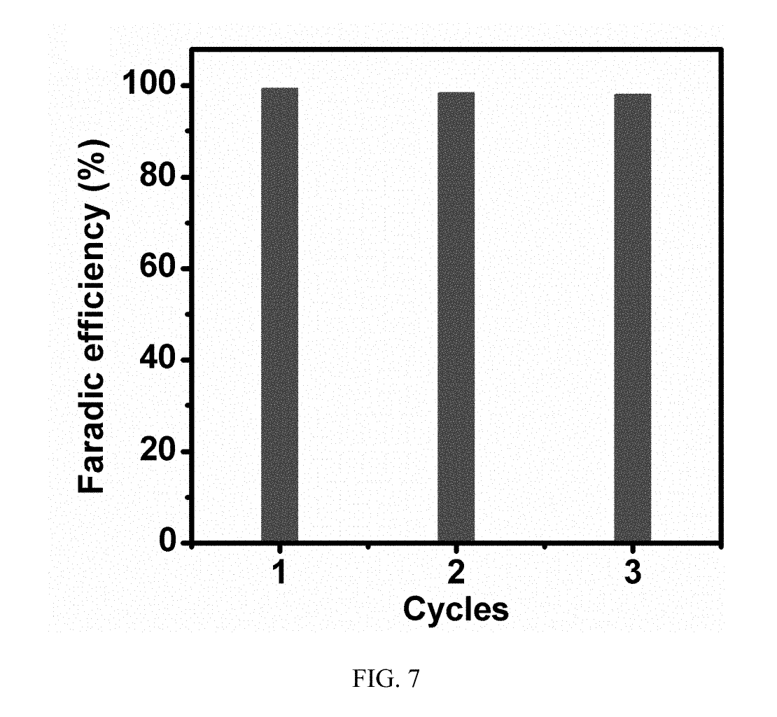

Generally, it is thought that there are two pathways for HMF oxidation: one is through an initial alcohol oxidation to form DFF as the intermediate (Scheme 1a), while the other is through an initial aldehyde oxidation to form HMFCA as the intermediate (Scheme 1b). Both pathways converge at the formation of FFCA prior to FDCA. In the present case, HMF oxidation catalyzed by Ni₂P NPA/NF likely followed the HMFCA route, as revealed by the relatively higher concentration of HMFCA compared to that of DFF (FIG. 6). This pathway is similar to the most reported aerobic oxidation reactions. However, the DFF route may be possible as DFF was also detected during electrolysis. The stability of Ni₂P NPA/NF for HMF oxidation was also investigated by performing three successive cycles of the above constant potential electrolysis utilizing the same Ni₂P NPA/NF. As shown in FIG. 7, the calculated Faradaic efficiencies of FDCA formation for these electrolysis trials were in the range of about 98-100%, illustrating the robust stability of Ni₂P NPA/NF for HMF oxidation.

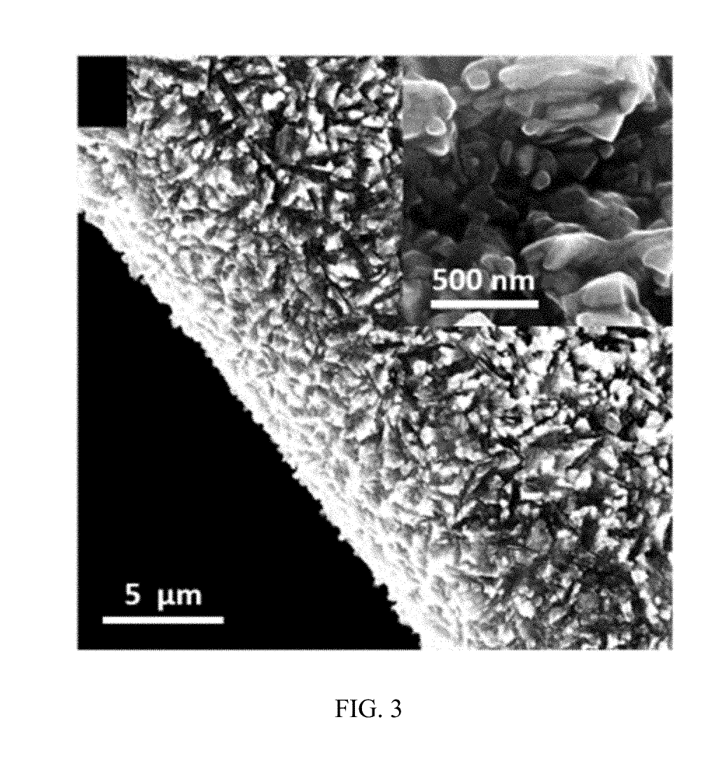

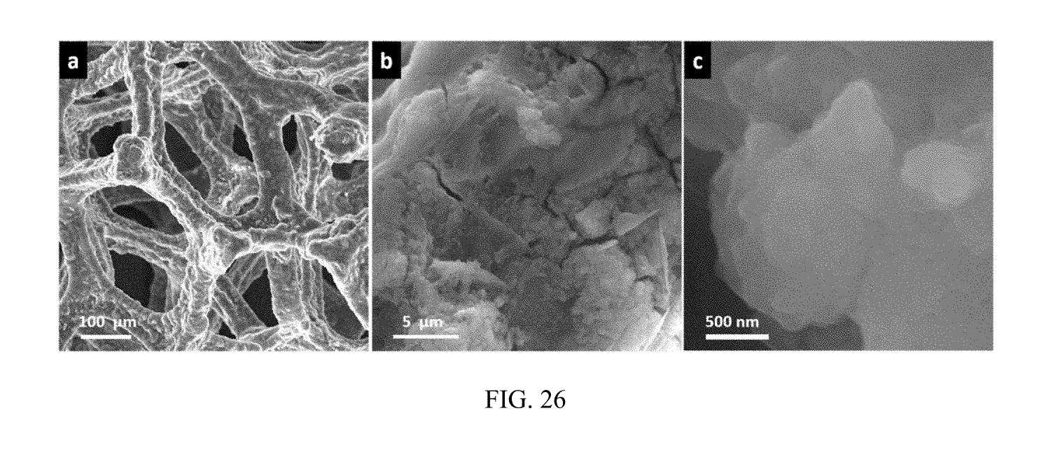

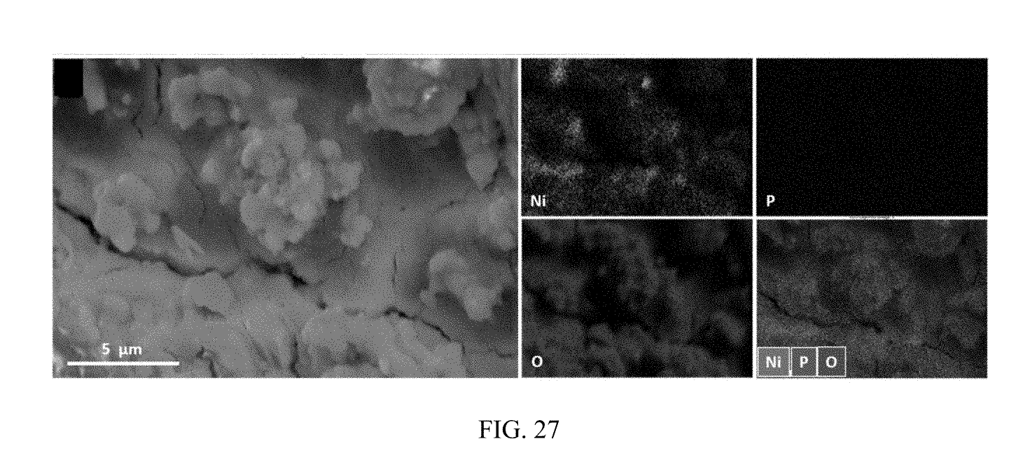

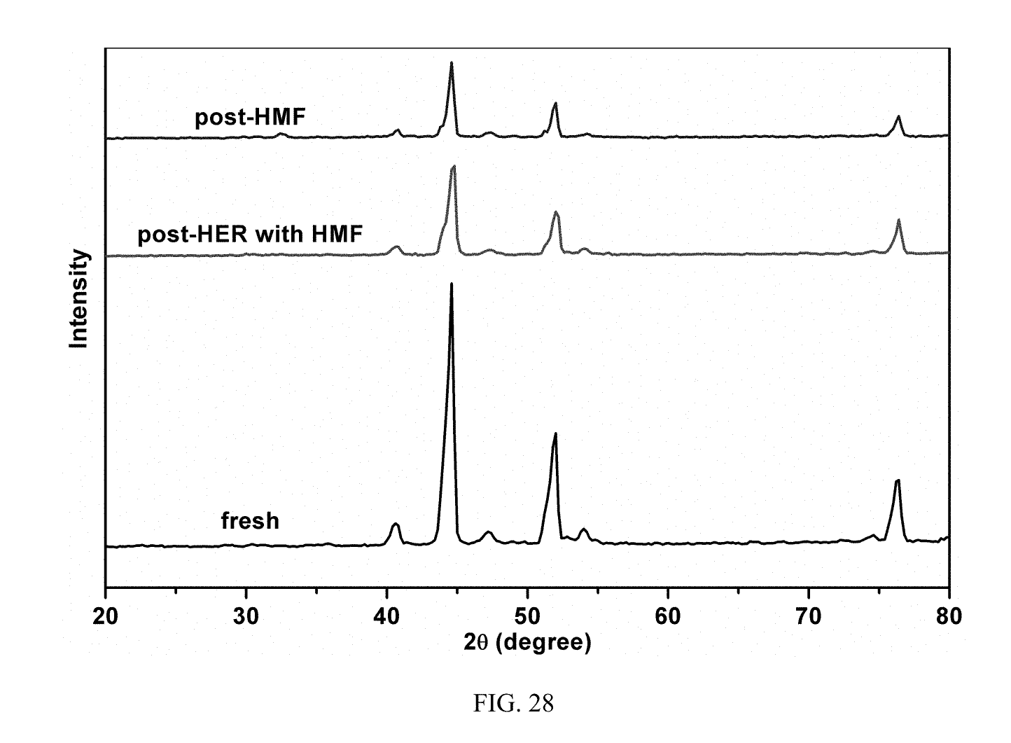

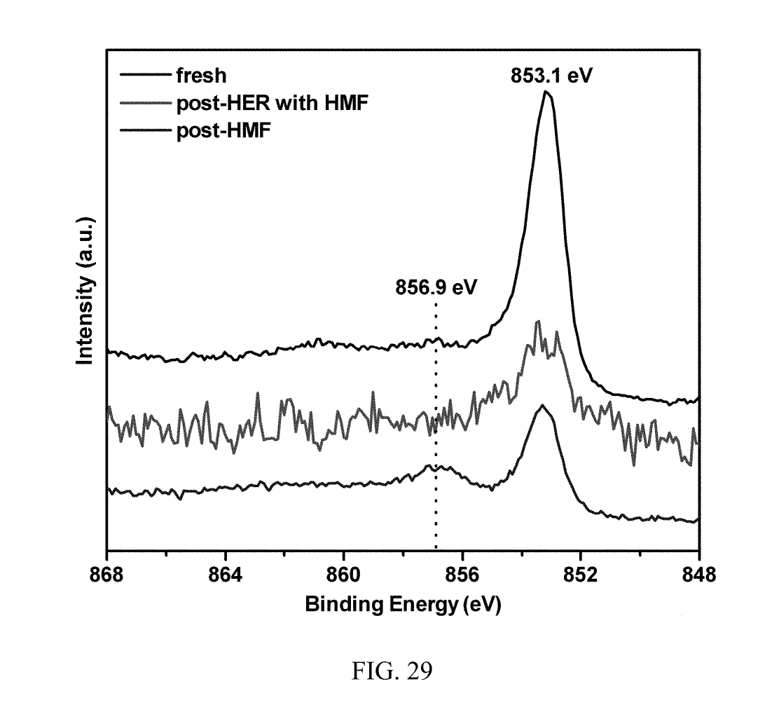

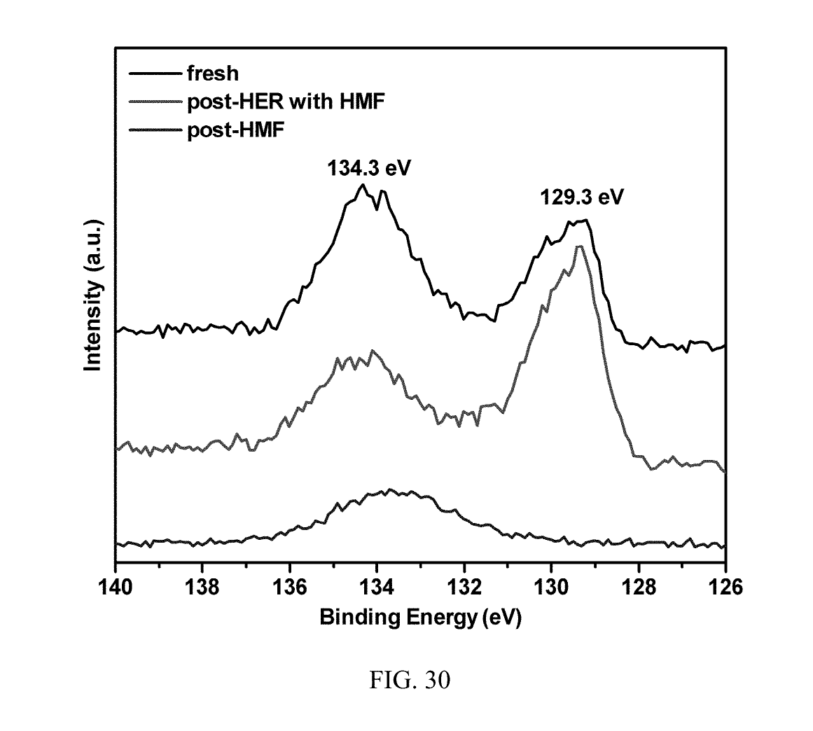

Although a low-magnified SEM image (FIG. 26a) and XRD pattern (FIG. 28) of Ni₂P NPA/NF after HMF oxidation (named as post-HMF Ni₂P NPA/NF) indicates inheritance of the overall 3D hierarchically porous configuration and primary Ni₂P phase, a close inspection of the post-HMF Ni₂P NPA/NF in high-magnified SEM images (FIG. 26b-c and FIG. 27) reveals the presence of featureless monoliths and cracks, different from the fresh sample (FIG. 3). Elemental mapping results (FIG. 27) demonstrate that the post-HMF Ni₂P NPA/NF was mainly composed of Ni and P, plus a large concentration of O over the newly formed monoliths. On the other hand, the high-resolution Ni 2P_{3/2} XPS spectrum of the post-HMF Ni₂P NPA/NF sample displayed an intensity decrease at about 853.1 eV (assignable to Ni δ⁺ in Ni₂P) while an increase at about 856.9 eV (corresponding to oxidized Ni species), confirming partial oxidation of Ni₂P (FIG. 29). This oxidation phenomenon was also revealed by the increased intensity of the peak ascribed to oxidized P species in the high-resolution P 2p XPS spectrum (FIG. 30). Taken together, it may be concluded that the real catalytically active sites for HMF oxidation reaction are the oxidized Ni species.

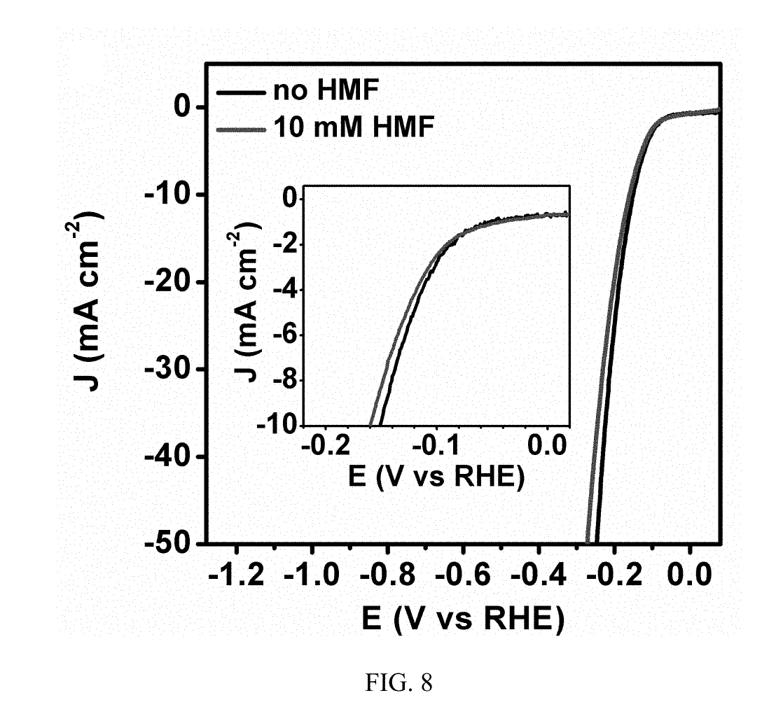

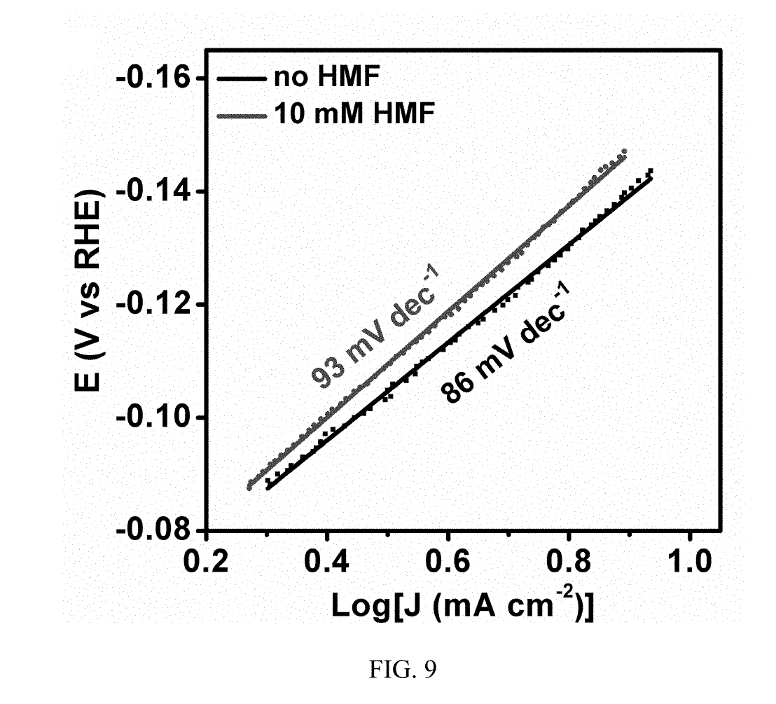

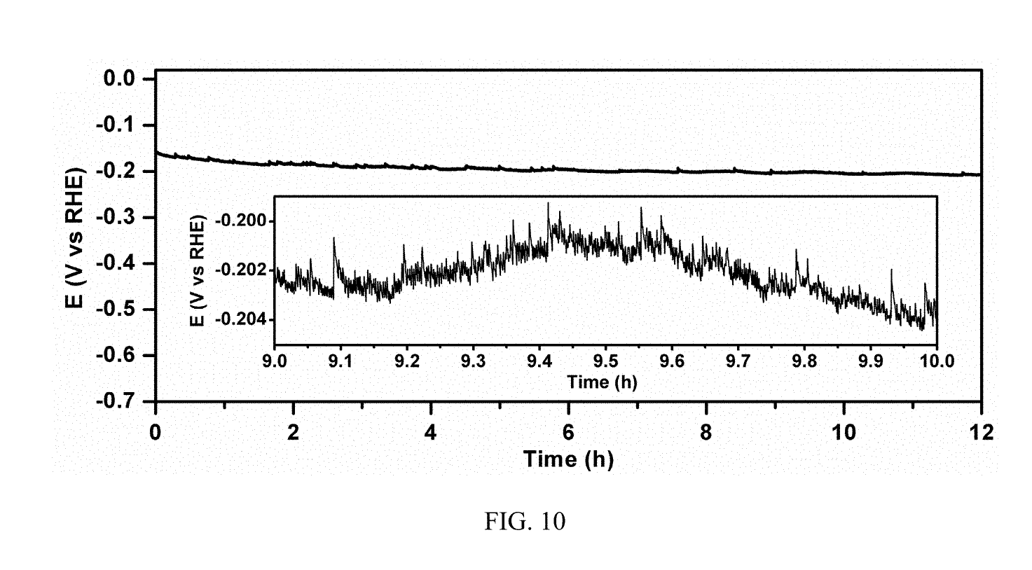

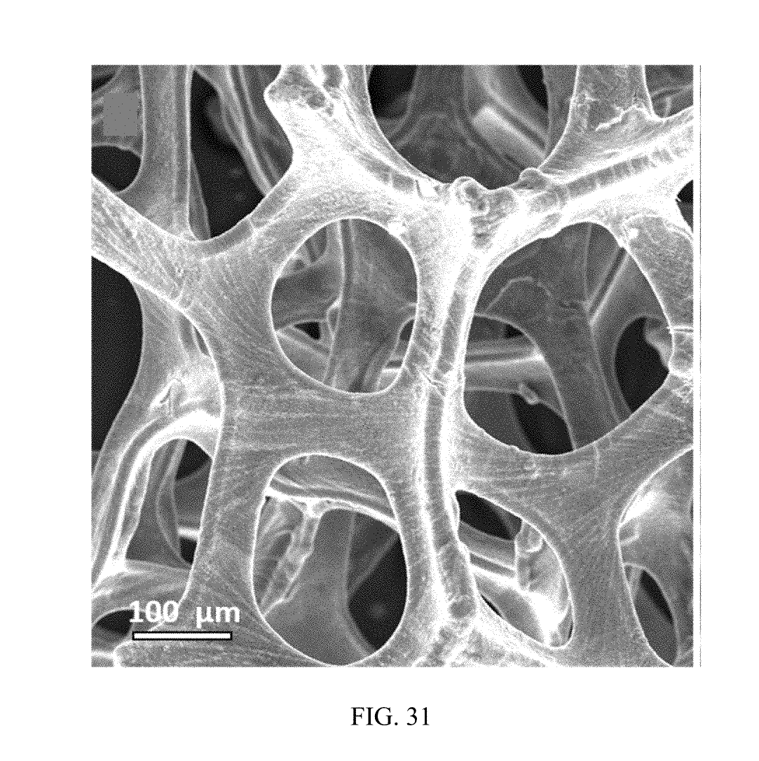

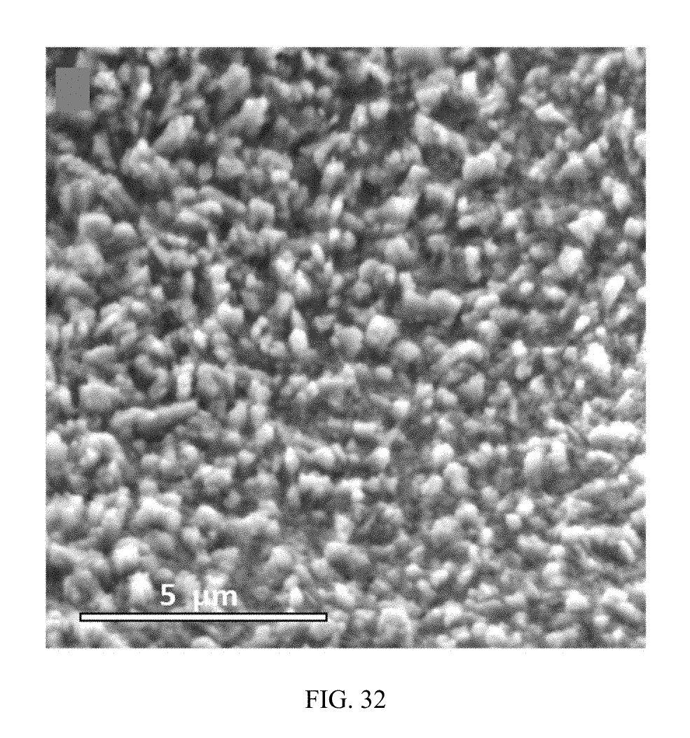

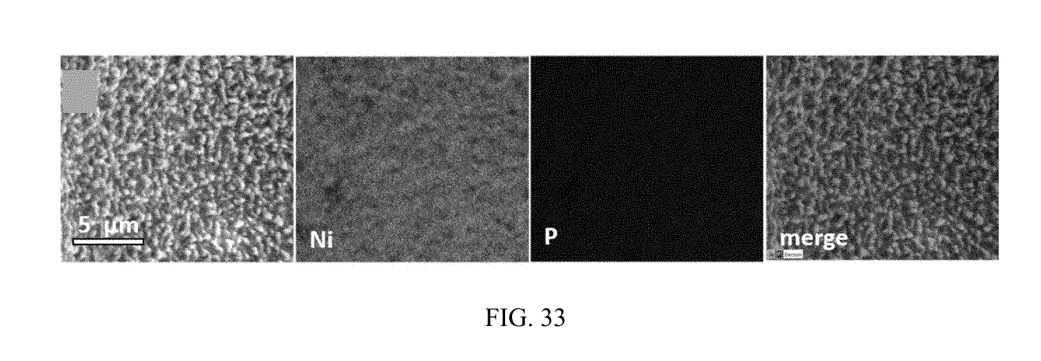

To couple HER and HMF oxidation for simultaneous H₂ and FDCA production, the Ni₂P NPA/NF electrocatalyst has to maintain excellent HER performance in the presence of HMF due to the potential permeation of HMF across the membrane from the anode compartment to the cathode site. Therefore, the impact of HMF on the HER activity of Ni₂P NPA/NF under the harshest condition (assuming all the HMF were present in the cathode compartment) was evaluated. As demonstrated in FIGS. 8 and 9, the HER LSV curves of Ni₂P NPA/NF in about 1.0 M KOH before and after the addition of about 10 mM HMF exhibited a small cathodic shift of about 9 mV to reach about −10 mA cm⁻², and the calculated Tafel slope only increased from about 86 to about 93 mV dec⁻¹ (FIG. 9). Besides, an about 12-h chronopotentiometry experiment conducted at a current density of about −10 mA cm⁻² in about 1.0 M KOH containing about 10 mM HMF demonstrated that the overpotential required to afford about −10 mA cm⁻² increased by less than about 45 mV (FIG. 10). The squiggle of an expanded chronopotentiometric curve may also imply the formation and release of H₂ bubbles on the catalyst surface (FIG. 10 inset). XRD (FIG. 28), XPS (FIGS. 29 and 30), and SEM (FIGS. 31-33) results of Ni₂P NPA/NF after the about 12-h HER stability test in about 1.0 M KOH with about 10 mM HMF (named as post-HER with HMF Ni₂P NPA/NF) confirms the retention of its morphology and composition. These results strongly suggest the negligible influence of HMF on the HER activity and the stability of Ni₂P NPA/NF under the current condition.

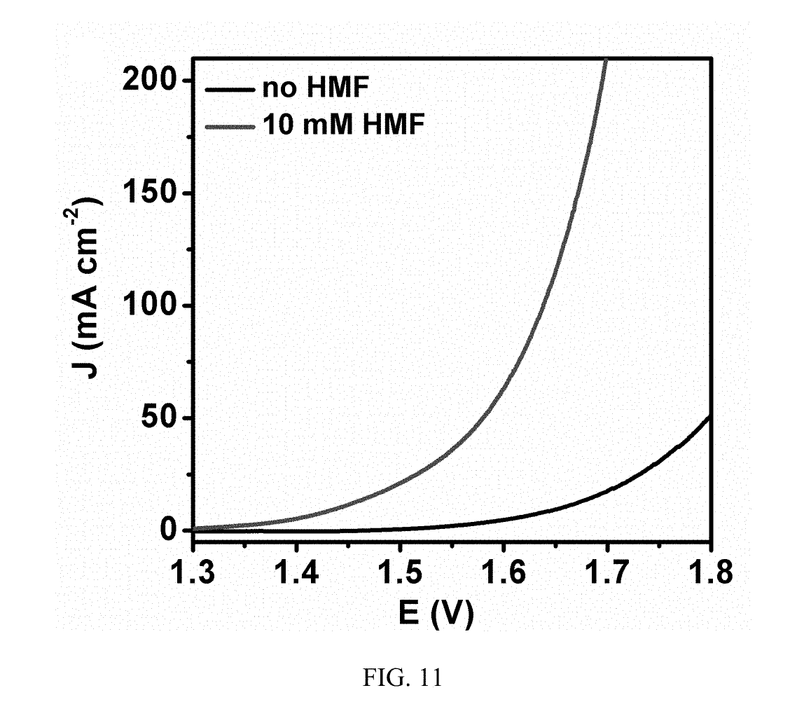

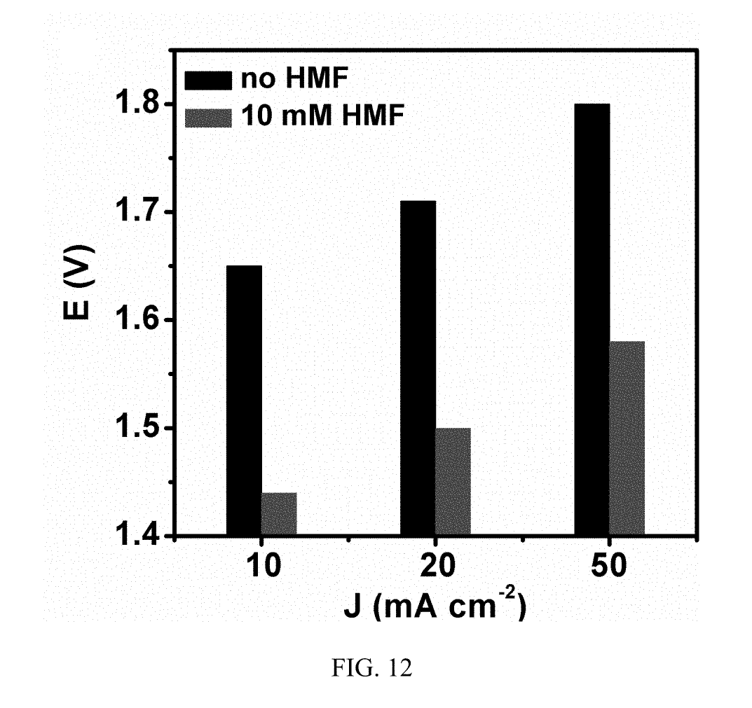

Given the aforementioned excellent electrocatalytic HER and HMF oxidation performance of Ni₂P NPA/NF in the same electrolyte (about 1.0 M KOH with about 10 mM HMF), an electrolyzer in a two-electrode configuration using Ni₂P NPA/NF as both anode and cathode electrocatalysts was assembled to achieve simultaneous H₂ and FDCA generation. For comparison, overall water splitting was also tested for a Ni₂P NPA/NF catalyst couple in the absence of HMF. As shown in FIG. 11, the Ni₂P NPA/NF couple needed cell voltages of only about 1.65 and about 1.80 V to afford about 10 and about 50 mA cm⁻², respectively, lower than or comparable to those of other nonprecious overall water splitting electrocatalysts, including Co—P (1.64 V for 10 mA cm⁻²), NiFe LDH/NF (1.70 V for 10 mA cm⁻²), Ni₃S₂/NF (1.76 V for 13 mA cm⁻²), and Ni₅P₄/NF (1.70 V for 10 mA cm⁻²), suggesting its exceptional performance for overall water splitting. Remarkably, after introducing about 10 mM HMF, the cell voltages to reach about 10 and about 50 mA cm⁻² were dramatically reduced to about 1.44 and about 1.58 V, respectively (FIG. 12), implying much better energy conversion efficiency of Ni₂P NPA/NF-catalyzed HER and HMF oxidation relative to sole water splitting.

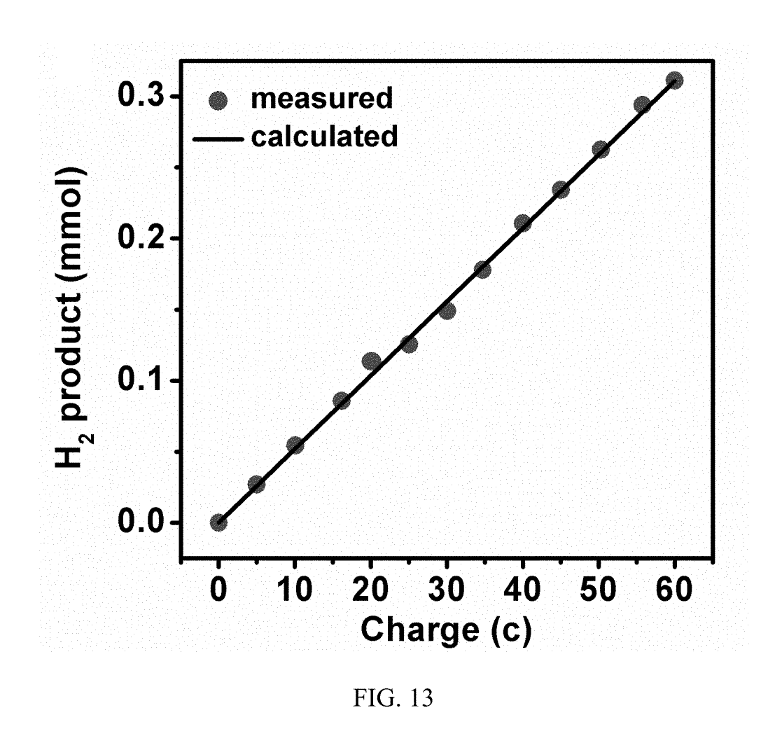

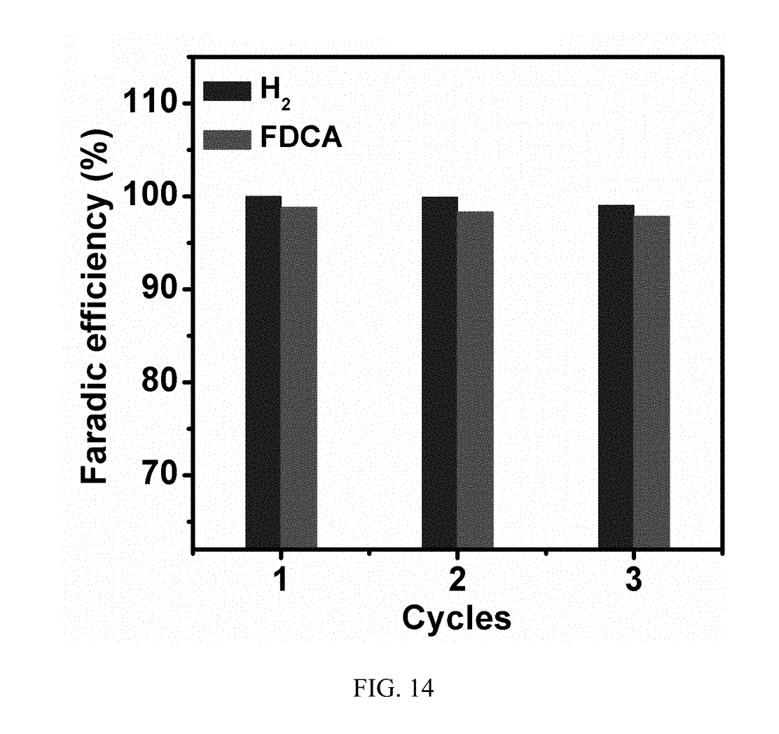

To quantify the produced H₂ and FDCA under a two-electrode configuration, a long-term electrolysis at a constant cell voltage of about 1.50 V vs RHE was performed to pass the charge of about 59 C. As shown in FIG. 13, the generated H₂ quantified by gas chromatography (GC) matched the calculated amount based on passed charge very well, leading to a Faradaic efficiency of about 100%. Analysis of the resulting electrolyte by HPLC resulted in an about 98% Faradaic efficiency for FDCA production. It should be noted that such a long-term controlled potential electrolysis was repeated three times for the same Ni₂P NPA/NF catalyst couple and no apparent decrease in Faradaic efficiencies was observed (FIG. 14), implying outstanding stability of Ni₂P NPA/NF for integrated HER and HMF oxidation.

In this exemplary example, a novel, facile, and efficient strategy is demonstrated for simultaneous H₂ production and biomass upgrading with Faradaic efficiencies of about 100% and about 98%, respectively, which may be achieved via a bifunctional Ni₂P NPA/NF electrocatalyst. Likely owing to more favorable thermodynamics and kinetics of HMF oxidation than OER catalyzed by Ni₂P NPA/NF, cell voltage to reach benchmark current densities (about 10, about 20, and about 50 mA cm⁻²) for H₂ production was significantly reduced by more than 200 mV, and concomitantly the oxidation product FDCA was more economically valuable than raw HMF as well as O₂ from pure water splitting. Taking into account the low cost for catalyst preparation, the high efficiency for the production of both H₂ and FDCA, as well as the abundance of substrates (H₂O and biomass), this strategy may be practical for future energy conversion technologies.

Chemicals: 5-Hydroxymethylfurfural (HMF) and 2,5-furandicarboxylic acid (FDCA) were purchased commercially. 2,5-Diformylfuran (DFF) and 2-formyl-5-furancarboxylic acid (FFCA) were purchased commercially. 5-Hydroxymethyl-2-furan-carboxylic acid (HMFCA) was purchased commercially. Potassium hydroxide and sodium hypophosphite monohydrate were purchased commercially. Nickel foam with purity greater than about 99.99% was purchased commercially. All chemicals may be used as received without any further purification. Water deionized (about 18 MΩcm⁻¹) with a Barnstead E-Pure system may be used in all experiments.

Synthesis of Ni₂P NPA/NF: A convenient and elegant approach was used to synthesize Ni₂P NPA/NF by direct phosphidation of commercially available nickel foam using sodium hypophosphite monohydrate (NaH₂PO₂.H₂O) as the phosphorous source. Typically, a piece of commercial nickel foam with a size of about 0.5 cm×about 0.5 cm was placed at the center of a tube furnace, and about 1.0 g NaH₂PO₂.H₂O was placed at the upstream side and near to the nickel foam. After flushed with Ar for about 30 min, the center of the furnace was heated to about 300° C. with a ramping rate of about 10° C. min⁻¹ and kept at about 300° C. for about 1 h to partially convert the metallic nickel to nickel phosphide. After the system was cooled to room temperature, the final product was obtained. Many variations on this procedure will be readily apparent to one of skill in the art.

Physical methods: The X-ray photoelectron spectroscopy samples were affixed on a stainless steel Kratos sample bar, loaded into the instrument's load lock chamber, and evacuated to about 5×10⁻⁸ torr before they were transferred into a sample analysis chamber under ultrahigh vacuum conditions (about 10⁻¹⁰ torr). X-ray photoelectron spectra were collected using monochromatic Al Kα source (about 1486.7 eV) at about a 300×700 μm spot size. Low resolution survey and high resolution region scans at the binding energy of interest were collected for each sample. To minimize charging, all samples were flooded with low-energy electrons and ions from the instrument's built-in charge neutralizer. The samples were also sputter cleaned inside the analysis chamber with about 1 keV Ar⁺ ions for about 30 seconds to remove adventitious contaminants and surface oxides. XPS energy corrections on high resolution scans were calibrated by referencing the C 1s peak of adventitious carbon to about 284.5 eV. The generated H₂ during electrolysis was quantified with an SRI gas chromatography system 8610C equipped with a Molecular Sieve 13×packed column, a HayesSep D packed column, and a thermal conductivity detector. The oven temperature was maintained at about 60° C. and argon was used as the carrier gas.

Electrocatalytic measurements: Electrochemical HER, OER, and HMF oxidation measurements were typically performed with a three-electrode cell configuration. The as-prepared Ni₂P NPA/NF was directly used as the working electrode, a Ag/AgCl (sat. KCl) electrode as the reference electrode, and a carbon rod as the counter electrode. All the potentials were quoted with respect to reversible hydrogen electrode (RHE) through RHE calibration according to the reported method (Nat. Mater. 2011, 10, 780, which is incorporated by reference herein in its entirety). The calibration was performed in the high-purity hydrogen saturated electrolyte (about 1.0 M KOH) with a Pt wire as the working electrode. Cyclic voltammetry was run at a scan rate of about 1 mV s⁻¹, and the average of the two potentials at which the current crossed zero was taken to be the thermodynamic potential for the hydrogen electrode reactions. The electrochemical HER, OER, and HMF oxidation experiments were conducted in about 10 mL of about 1.0 M KOH solution with and without about 10 mM HMF. For two-electrode electrolysis, Ni₂P NPA/NF were employed as both anode and cathode. The potential range was cyclically scanned at a scan rate of about 2 mV s⁻¹. iR (current times internal resistance) compensation was applied in all the electrochemical experiments to account for the voltage drop between the reference and working electrodes. The stability tests of Ni₂P NPA/NF for HMF oxidation and two-electrode electrolysis were evaluated by chronoamperometry at about 1.423 V vs RHE and cell voltage of about 1.50 V, respectively, in about 10 mL mixed solution of about 1.0 M KOH with about 10 mM HMF for three successive cycles.

Quantitative product analysis: To analyse the products of HMF oxidation quantitatively and calculate the corresponding Faradaic efficiencies, about 10 pL of the electrolyte solution during chronoamperometry testing at about 1.423 V vs RHE (for the three-electrode configuration) or cell voltage of about 1.50 V (for the two-electrode configuration) were taken from the cell and diluted with about 490 pL water, which were then analysed using HPLC with an ultraviolet-visible detector set at about 265 nm and an about 4.6 mm×about 150 mm Shim-pack GWS about 5 μm C18 column. Solvent A was about 5 mM ammonium formate aqueous solution and solvent B was methanol. Separation was accomplished using an isocratic elution by using about 30% B during about 10 min run time and the flow rate was set at about 0.5 mL/min. The quantification of each analyte was determined based upon the calibration curve obtained from the standard solutions of known concentrations.

The HMF conversion (%) and the yields (%) of oxidation products were calculated using equations (1) and (2):

$$HMF conversion ( \% ) = \frac{mol of HMF consumed}{mol of initial HMF} \times 100$$

$$products yield = \frac{mol of product formed}{mol of initial HMF} \times 100$$

The Faradaic efficiency of FDCA formation was calculated using the equation (3):

$$FE ( \% ) = \frac{mol of FDCA formed}{total charge passed / ( 6 \times F )} \times 100$$

where F is the Faraday constant (96,485 C mol⁻¹).

Catalyst synthesis: Two approaches were adopted to synthesize transition metal-based catalysts for coupling 5-hydroxylmethyl furfural (HMF) or furfural oxidation with hydrogen production from water splitting.

The first one was electrodeposition. For instance, Co—P coated on copper foam (Co—P/CF) can be synthesized according to the following procedure. Prior to electrodeposition, copper foams were rinsed with water and ethanol thoroughly to remove residual organic species. Copper foam with a geometric area of about 0.25 cm² (about 4 cm² for samples of electrolysis experiments) was exposed to deposition solution (about 50 mM CoSO₄ and about 0.5 M NaH₂PO₂ in water). A platinum wire was used as the counter electrode and a Ag/AgCl (sat. KCl) electrode as the reference electrode. Nitrogen was bubbled through the electrolyte solution for at least about 20 min prior to deposition and maintained during the entire deposition process. The potential of consecutive linear scans was cycled about 15 times between about −0.3 and about −1.0 V vs Ag/AgCl at a scan rate of about 5 mV/s under stirring. After deposition, the copper foam was removed from the deposition bath and rinsed with copious water gently. The as-prepared Co—P/CF can be directly used for electrochemical experiments or stored under vacuum at room temperature for future use. A similar method can be utilized to prepare Fe—P/CF and Ni—P/CF catalysts with iron or nickel salt as the metal source, respectively.

The second approach was annealing under targeted gas atmosphere. For instance, Ni₂P coated on nickel foam (Ni₂P/NF) can be prepared via direct phosphidation of commercially available nickel foam using sodium hypophosphite monohydrate (NaH₂PO₂.H₂O) as the phosphorous source. Typically, a piece of commercial nickel foam with a size of about 0.5 cm×about 0.5 cm was placed at the center of a tube furnace, and about 3 g NaH₂PO₂.H₂O was placed at the upstream side and near to the nickel foam. After flushed with Ar for about 30 min, the center of the furnace was heated to about 300° C. with a ramping rate of about 10° C. min⁻¹ and kept at about 300° C. for about 1 h to partially convert the metallic nickel to nickel phosphide. After the system cooled down to room temperature, the final product was obtained. A similar method can be applied to prepare Ni₃S₂/NF and Ni₃N/NF with sulfur or ammonia as the nonmetal element source, respectively.

### Example 2

### Electrocatalysis of Furfural Oxidation Coupled with H₂ Evolution via Nickel-Based Electrocatalysts in Water

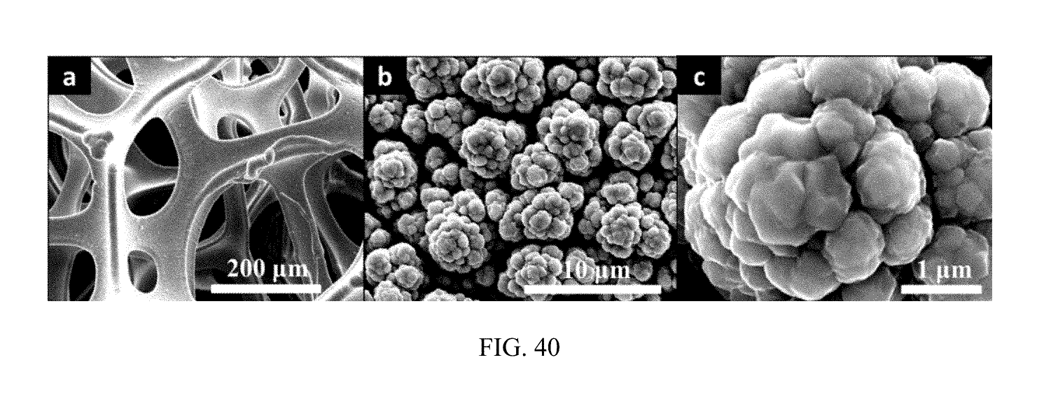

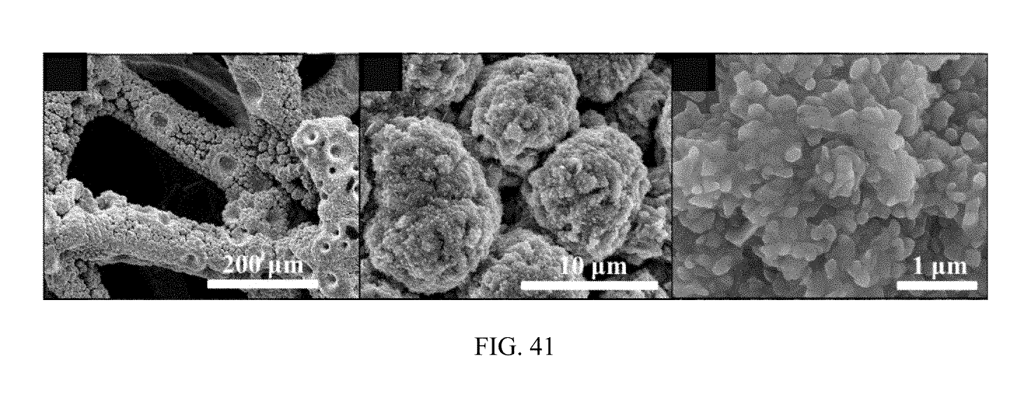

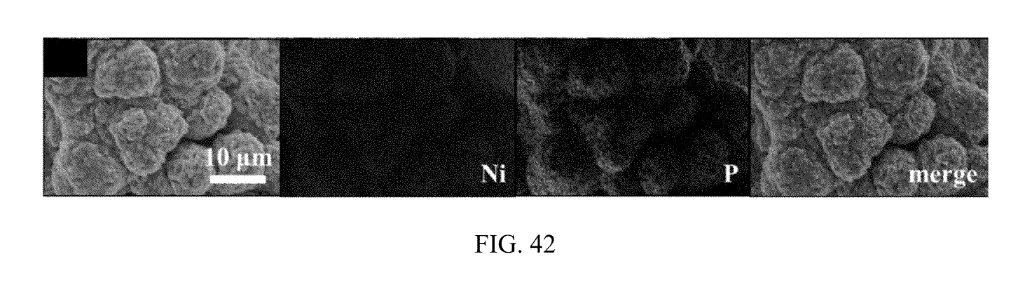

Ni₂P/Ni/NF was prepared. Commercially available nickel foam has a porous structure with smooth skeleton as shown in FIG. 40a. After electrodeposition of nickel, fine nickel particles (diameter of about 0.5—about 1μm) were grown on the nickel foam (Ni/NF, FIG. 40 b-c), in sharp contrast to the smooth surface of the pristine nickel foam (FIG. 40a). Upon low-temperature phosphidation, the metallic Ni transformed to Ni₂P (FIG. 41), which could be confirmed by their XRD patterns. The corresponding elemental mapping images of Ni and P in Ni₂P/Ni/NF demonstrated a uniform distribution of Ni and P throughout the whole sample (FIG. 42), in agreement with the successful conversion from Ni/NF to Ni₂P/Ni/NF post phosphidation. X-ray photoelectron spectroscopy (XPS) was conducted to probe the composition of Ni₂P/Ni/NF and the valence state of each element. The XPS survey spectrum exhibits all the anticipated elements including Ni and P. The high-resolution Ni 2p XPS spectrum could be deconvoluted to peaks at about 853.2, about 855.6, and about 860.9 eV, which may be assigned to Ni δ⁺ in Ni₂P, oxidized Ni species, and the Ni 2p_{3/2} satellite peak of Ni₂P, respectively. The deconvolution of the high-resolution P 2p XPS spectrum led to a prominent peak in the region of about 129—about 130 eV, which could be attributed to the phosphide signal. The other peak around about 134.4 eV was due to the oxidized phosphorus species on surface because of exposure to air prior to the XPS measurement. Overall, Ni had partial positive charge (δ⁺) while P showed partial negative charge (δ⁻) in Ni₂P/Ni/NF, which is indicative of the transfer of electron density from Ni to P.

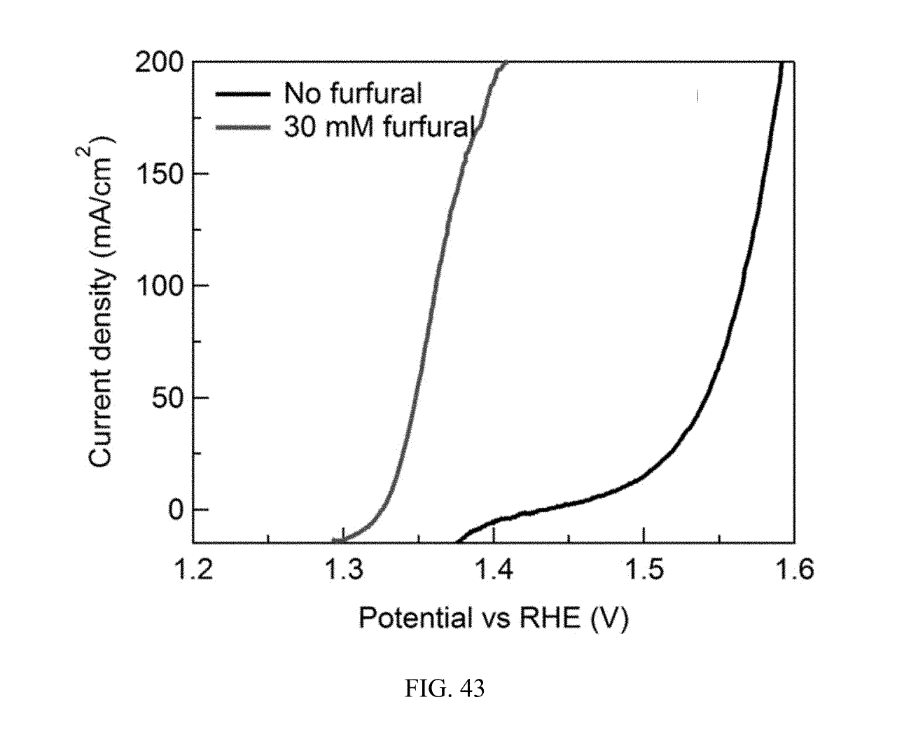

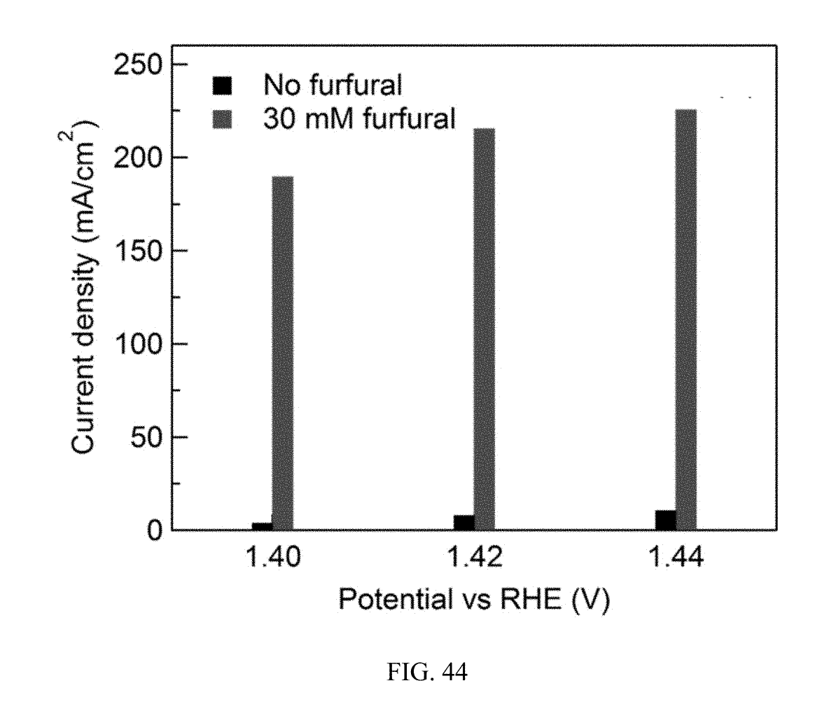

Since Ni₂P is an effective electrocatalyst for H₂ evolution in water, it was determined whether Ni₂P was able to catalyze the furfural oxidation in alkaline electrolyte (about 1.0 M KOH). All the electrochemical experiments in this example were conducted in a three-electrode configuration with a two-compartment cell separated by an anion-exchange membrane, unless otherwise noted. For most organic oxidation reactions in water, O₂ evolution could potentially be a competing reaction. Therefore, it was important to check the oxidation current in the absence of organic substrates. As shown in FIG. 43, the cyclic voltammogram of Ni₂P/NUNF in about 1.0 M KOH exhibited a catalytic oxidation current at an onset of about 1.55 V vs RHE (reversible hydrogen electrode). In order to avoid the interference of the redox feature of the nickel catalyst itself, only cathodic scan of the cyclic voltammogram was plotted in FIG. 43. Further scanning towards more positive potential produced a dramatic increase in current density accompanied with vigorous O₂ bubble formation on the electrode surface. Upon the addition of about 30 mM furfural, a catalytic current was observed at a much smaller potential. The catalytic onset cathodically shifted to about 1.34 V vs RHE, which is indicative of oxidation of furfural to 2-furoic acid (Scheme 2) relative to water oxidation. Rapid catalytic current rise was obtained when scanning towards more position potential. In fact, a current density of about 200 mA/cm² was achieved at about 1.41 V vs RHE, nearly about 180 mV smaller than that of pure water oxidation to reach the same current density. As demonstrated in FIG. 44, the current densities at about 1.40, about 1.42, and about 1.44 V vs RHE in the presence of furfural were at least about 20 times larger than those in the absence of furfural.

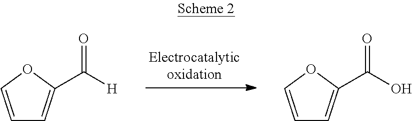 [SMILES: O=Cc1ccco1 ; O=C(O)c1ccco1]

In order to track the conversion of furfural and the yield of 2-furoic acid, a long-term chronoamperometry of furfural oxidation catalyzed by Ni₂P/NUNF was conducted at a constant potential of about 1.423 V vs RHE, as it was prior to the catalytic onset of water oxidation (FIG. 43). Since the oxidation of furfural to 2-furoic acid is a two-electron process, it was calculated that about 116 C was required to transform about 30 mM furfural (about 20 mL) completely to 2-furoic acid if a 100% Faradaic efficiency was assumed.

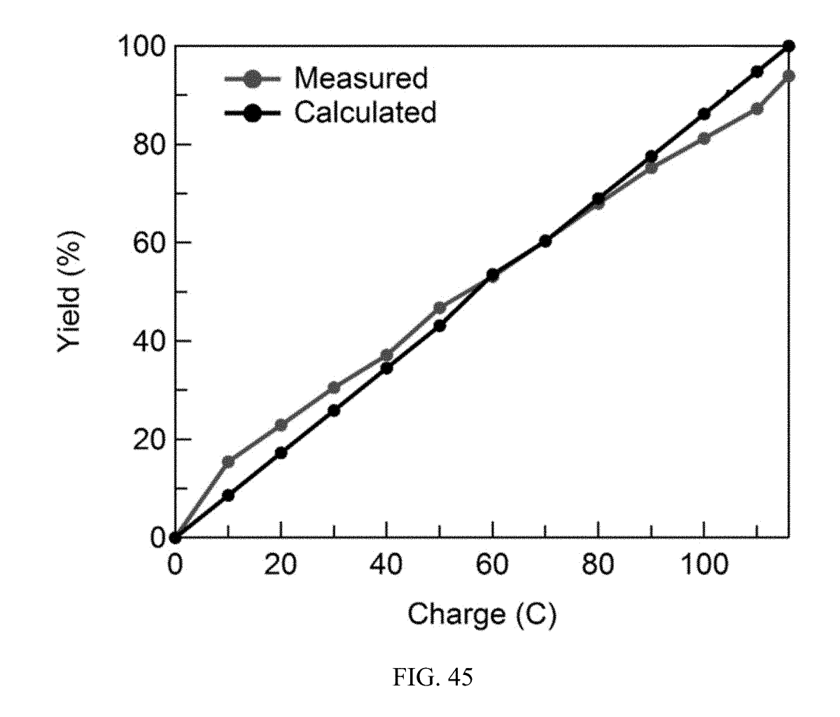

The concentration change of furfural and 2-furoic acid were quantified via high-performance liquid chromatography (HPLC) according to pre-established calibration curves. As shown in FIG. 45, the concentration of furfural decreased along with the increasing concentration of 2-furoic acid as more charge was consumed over time, suggesting the continuous conversion of furfural into 2-furoic acid during the electrocatalytic process. After about 116 C charge was consumed, nearly complete conversion of furfural (about 98%) was obtained with an about 94% yield of 2-furoic acid.

The robustness of Ni₂P/NUNF was assessed by consecutive oxidation electrolysis with the same concentration of furfural (about 30 mM) in fresh about 1.0 M KOH electrolyte and the same catalyst. The yields of 2-furoic acid of three continuous cycles were plotted, with all the yields falling in the range of about 96% to about 98%, demonstrating the remarkable stability of Ni₂P/NUNF for furfural oxidation under alkaline condition.

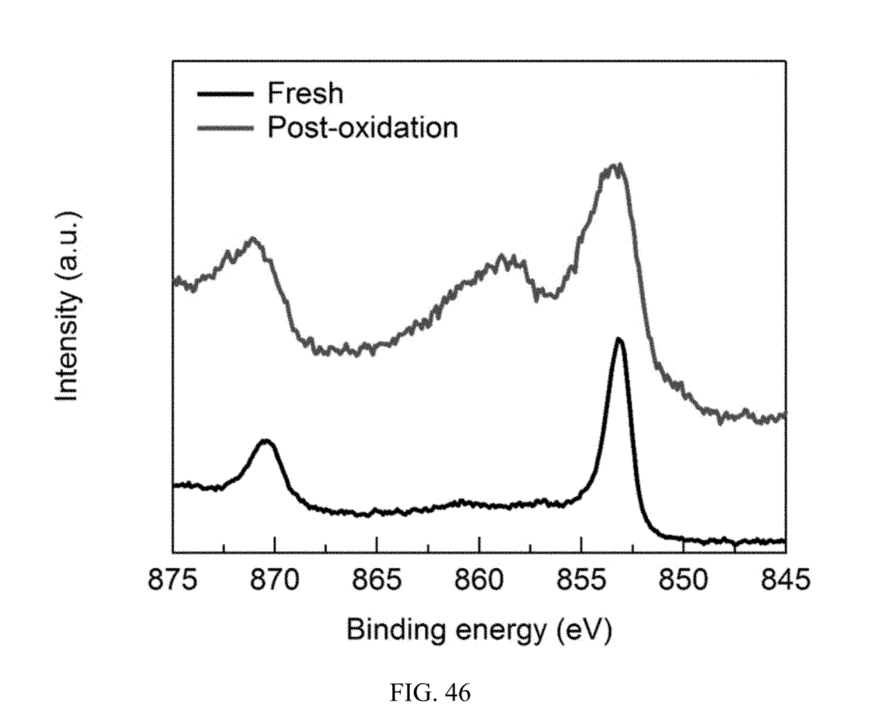

Post-electrolysis analysis was also performed for Ni₂P/NUNF to shed light on its morphology and composition change. The low-magnification SEM image showed that the post-furfural-oxidation Ni₂P/NUNF inherited the overall 3D porous structure. However, a close inspection of its high-magnification SEM images revealed the presence of featureless monoliths in addition to urchin-like microparticles, which was quite different from the fresh (FIGS. 40-42) and the post-HER samples. Elemental mapping results demonstrated that the post-furfural-oxidation Ni₂P/NUNF mainly consisted of Ni and P, plus a large concentration of O. Indeed, the high-resolution Ni 2p XPS spectrum of the post-furfural-oxidation Ni₂P/NUNF displayed a new peak at about 858.8 eV (FIG. 46 top), which could be attributed to nickel oxides formed during the electrocatalysis of furfural oxidation.

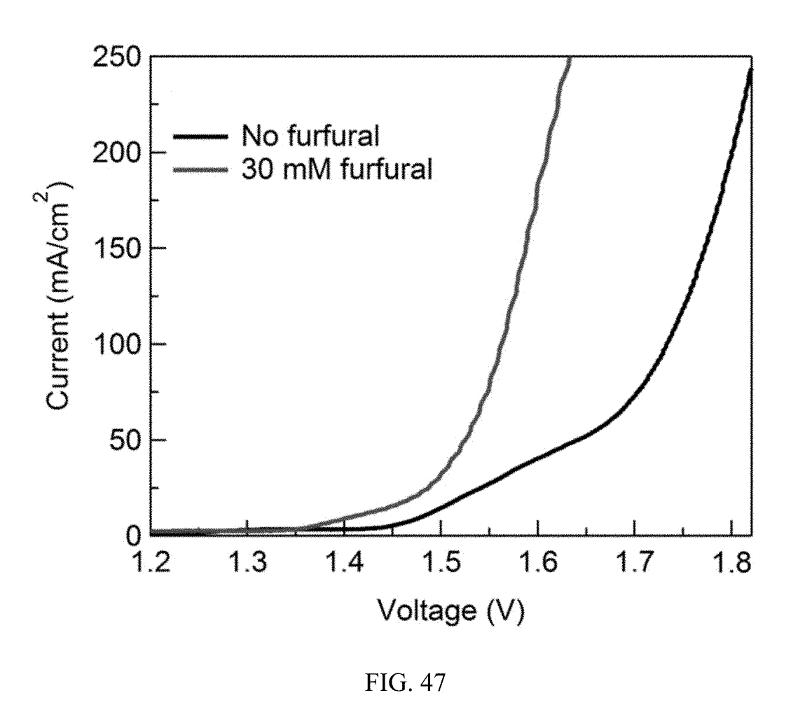

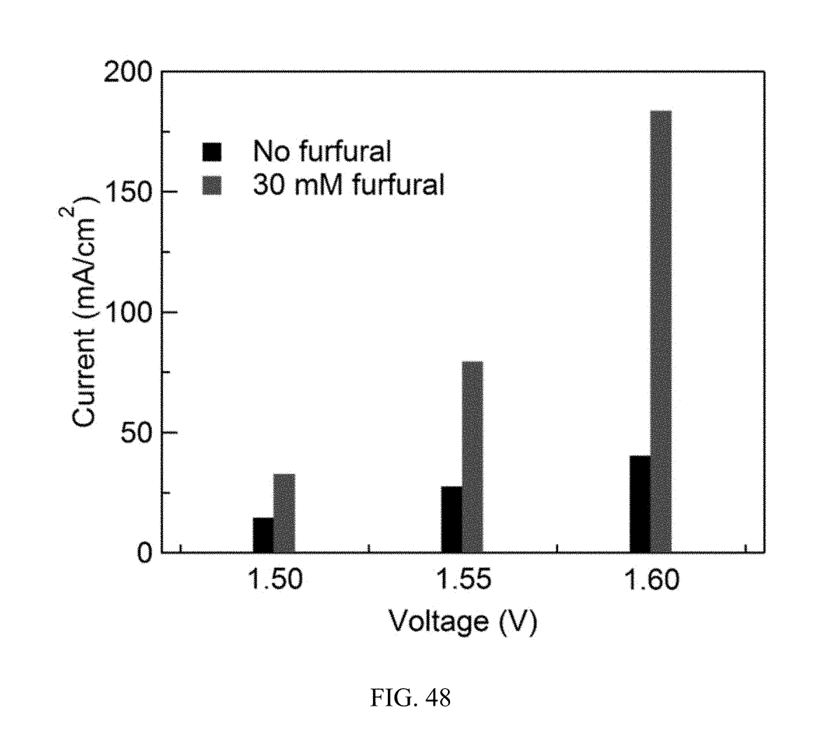

In view of these results, it was likely that Ni₂P/NUNF was able to catalyze both HER and furfural oxidation simultaneously. Hence, a two-electrode electrolyzer employing Ni₂P/NUNF as both the anode and cathode catalysts was constructed. The two compartments of this electrolyzer were separated by an anion exchange membrane and about 1.0 M KOH was used as the electrolyte. As a comparison, pure water splitting electrolysis was also assessed. In the absence of furfural in the anodic compartment, the Ni₂P/Ni₂P couple was able to catalyze overall water splitting to produce H₂ and O₂ (FIG. 47). It required a voltage of about 1.59 V to produce a catalytic current density of about 10 mA/cm². In sharp contrast, upon addition of about 30 mM furfural in the anodic compartment, the Ni₂P/NUNF catalyst couple exhibited a catalytic current at an onset potential less than about 1.4 V (FIG. 47). Only about 1.48 V was required to achieve the current density of about 10 mA/cm², about 110 mV smaller than that of sole water splitting. FIG. 48 compares the produced catalytic current densities of our Ni₂P/NUNF catalyst couple in the presence and absence of furfural at three different voltages, about 1.50, about 1.55, and about 1.60 V. The integrated HER and furfural oxidation system shows substantially higher catalytic current densities relative to those of only water splitting electrolysis, highlighting the improved return of voltage input of the former electrocatalytic coupling strategy.

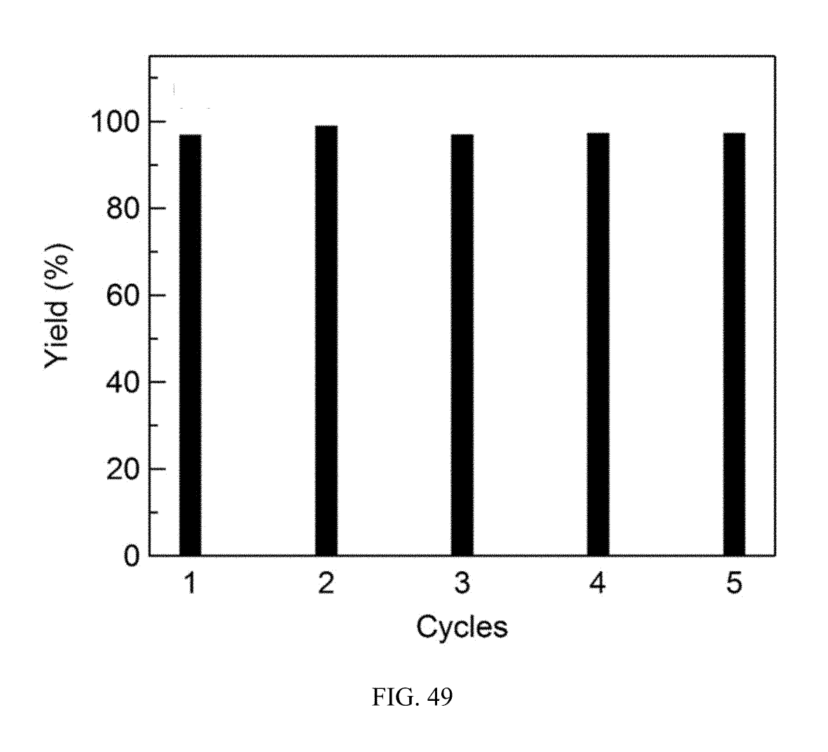

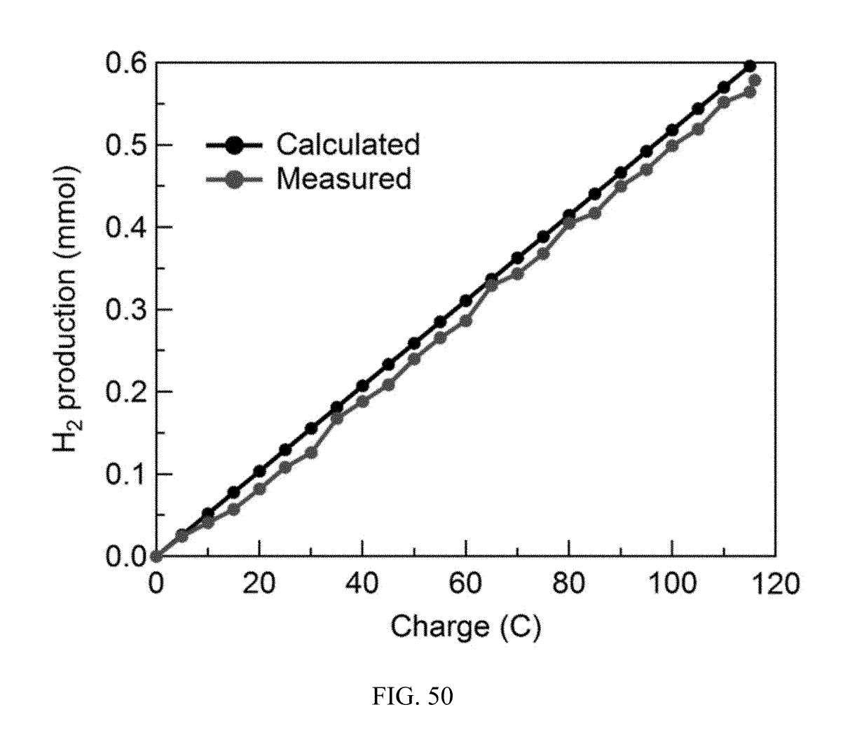

Furthermore, the robustness of the Ni₂P/NUNF catalyst couple for this integrated electrolysis was evaluated by five successive electrolysis cycles using the same catalyst couple and fresh about 1.0 M KOH electrolyte containing about 30 mM furfural for each cycle. As plotted in FIG. 49, the yields of 2-furoic acid were maintained in the range of about 97-99%, suggesting the strong robustness of the Ni₂P/NUNF catalyst couple for this integrated electrolysis. In addition, nearly unity Faradaic efficiency was also obtained for the cathodic H₂ evolution reaction (FIG. 50), as confirmed by a nearly overlap of gas chromatography-measured H₂ quantity and the calculated amount based on passed charge during electrolysis. Post-electrolysis analysis was also carried out on the N₂P/NUNF catalyst couple. The SEM and elemental mapping results of Ni₂P/NUNF as cathode and anode electrocatalysts displayed similar results as those observed for the corresponding half reactions discussed previously.

Disclosed by this example is a highly efficient electrochemical process employing Ni₂P/NUNF as a bifunctional electrocatalyst for the integrated H₂ production and furfual oxidation to 2-furoic acid. Ni₂P/NUNF solely consists of earth-abudant elements and it can be synthesized in a facile manner, suggesting low cost for large-scale manufacture. When acting as the electrocatalysts for both cathode and anode, Ni₂P/NUNF demonstrated excellent reactivty, strong robustness, and nearly unity Faradaic efficiencies for both H₂ production and 2-fuoric acid formation. High current density (i.e., about 250 mA/cm²) can be achieved at applied voltages much smaller than that of pure water splitting, manifesting its enhanced energy conversion efficiency. In addition, since furfural oxidation is only one of many potential organic oxidation reactions, such an intergrated strategy of H₂ production and organic oxiation will be able to be applied to a wide variety of organic transformations, resulting in numerous value-added products at the anode, instead of O₂.

Chemicals: Nickel chloride hexahydrate (NiCl₂.6H₂O), ammonium chloride (NH₄Cl), sodium hypophosphite monohydrate (NaH₂PO₂.H₂O), and potassium hydroxide (KOH) were all purchased from commercial vendors and used directly without any further purification. Furfural and 2-furoic acid were purchased from commercial venders and used as received. Nickel foam with purity greater than about 99.99% was purchased from a commercial vendor. Water was deionized (about 18 Ω·cm) using a Barnstead E-Pure system.

Synthesis of Ni/NF: The preparation of Ni/NF was conducted via chronopotentiometry. Prior to electrodeposition, Ni foam was sonicated in about 1.0 M HCl for about 10 min to remove residual organic species. Typically, the electrodeposition of 3D porous Ni nanoparticles on nickel foam (Ni/NF) was performed in a standard two-electrode system at about room temperature with an electrolyte consisting of about 2.0 M NH₄Cl and about 0.1 M NiCl₂. A piece of nickel foam with a size of about 0.5 cm×about 1 cm was used as the working electrode and a Pt wire as the counter electrode. The electrodeposition was carried out at a constant current of about −1.0 A/cm² for about 500 s to obtain Ni/NF samples. Many variations on these procedures will be readily apparent to one of skill in the art.

Synthesis of Ni₂P/NiNF: The resulting Ni/NF was placed at the center of a tube furnace, and about 1.0 g NaH₂PO₂.H₂O was placed at the upstream side and near Ni/NF. After flushed with Ar for about 20 min, the center of the furnace was quickly elevated to the reaction temperature of about 400° C. with a ramping rate of about 10° C./min and kept at about 400° C. for about 2 h to convert the metallic nickel to nickel phosphides. Many variations on these procedures will be readily apparent to one of skill in the art.

Physical Methods: XPS samples were affixed on a stainless steel Kratos sample bar, loaded into an instrument's load lock chamber, and evacuated to about 5×10⁻⁸ torr before they were transferred into a sample analysis chamber under ultrahigh vacuum conditions (about 10⁻¹⁰ torr). X-ray photoelectron spectra were collected using the monochromatic Al Kα source (about 1486.7 eV) at an about 300×about 700 μm spot size. High resolution regions at the binding energy of interest were taken for each sample. The samples were also sputter cleaned inside the analysis chamber with about 1 keV Ar⁺ ions for about 30 s to remove adventitious contaminants. Energy corrections for high resolution spectra were calibrated by referencing the C 1s peak of adventitious carbon to about 284.5 eV.

Electrocatalytic Measurements: Electrochemical experiments were performed with a three-electrode configuration. Aqueous Ag/AgCl reference electrodes (saturated KCl) were purchased commercially. The reference electrode in aqueous media was calibrated with ferrocenecarboxylic acid whose Fe^{3+/2+} couple is about 0.284 V vs SCE. All potentials reported in this example were converted from vs Ag/AgCl to vs RHE (reversible hydrogen electrode) by adding a value of about 0.197 30 0.059×pH. iR (current times internal resistance) compensation was applied in polarization and controlled potential electrolysis experiments to account for the voltage drop between the reference and working electrodes. The catalyst-coated NF was directly used as the working electrode. A Pt wire was used as the counter electrode. All the electrochemical measurements were conducted in about 1.0 M KOH with a two-compartment cell in which the anode and cathode compartments were separated by an anion exchange membrane (Fumasep FAA-3-PK-130) purchased commercially.

Quantitative Product Analysis: In order to analyze the product and furfural oxidation quantitatively, about 100 μL of the electrolyte solution was periodically collected from the electrolyte solution during chronoamperometry and diluted with about 900 μL water. Subsequently, the aforementioned solutions were further diluted 3 times with water. The final samples were then analyzed by HPLC at about room temperature to calculate the furfural conversion and the quantity of 2-furoic acid. The HPLC instrument was equipped with an ultraviolet-visible detector set at about 265 nm and an about 4.6 mm×about 150 mm Shim-pack GWS 5μm C18 column. The eluent solvent was a mixture of about 5 mM ammonium formate aqueous solution and methanol. Separation was accomplished using an isocratic elution by using about 50% ammonium formate aqueous solution and about 50% methanol for about 10 min with the flow rate set at about 0.5 mL mid'. The quantification of furfural and its oxidation product was calculated based on the calibration curves of standard compounds.

### Example 3

### Efficient H₂ Evolution Coupled with Oxidative Refining of Alcohols via a Hierarchically Porous Nickel Bifunctional Electrocatalyst

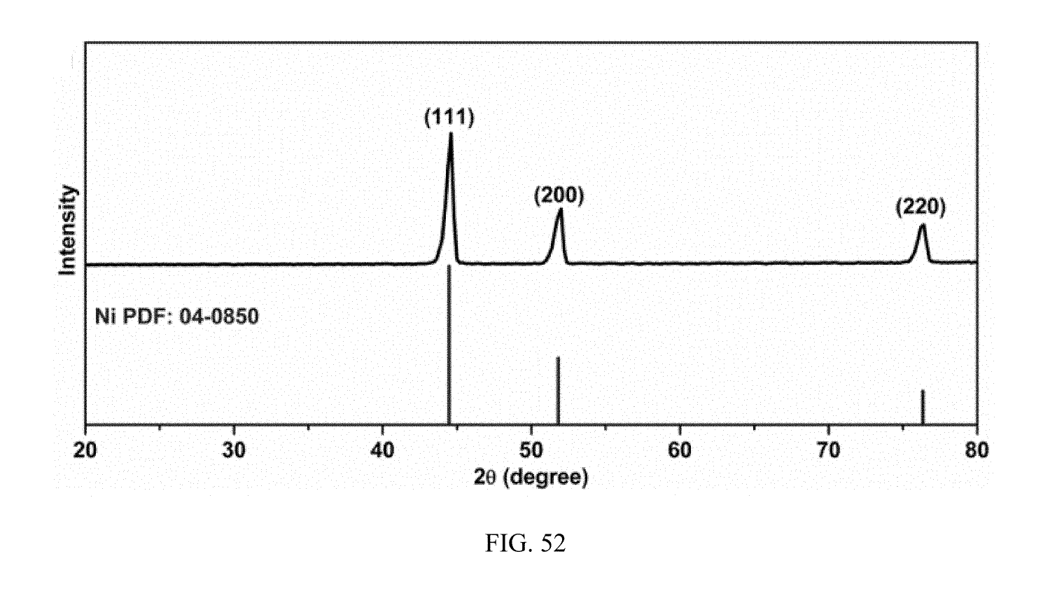

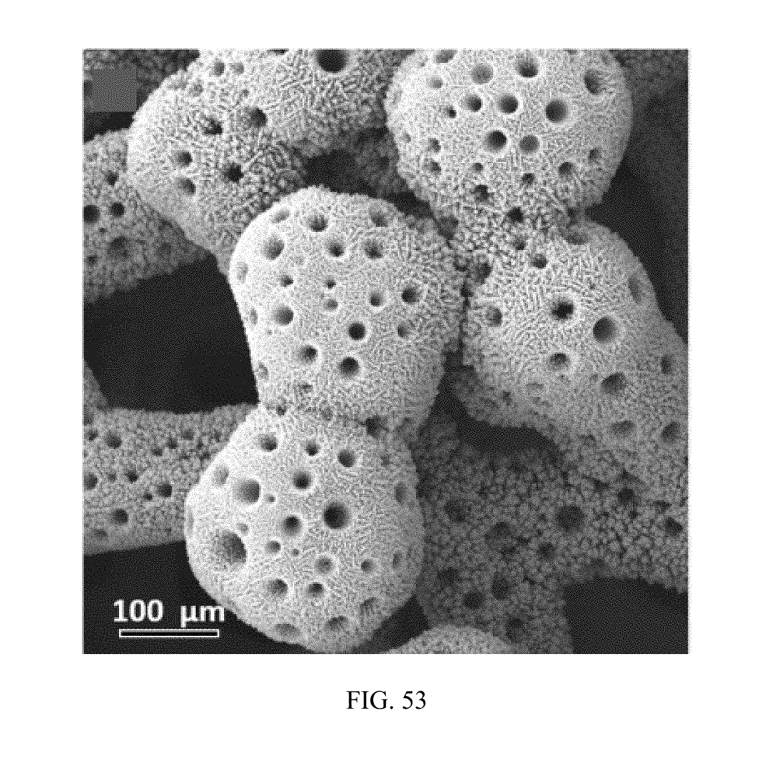

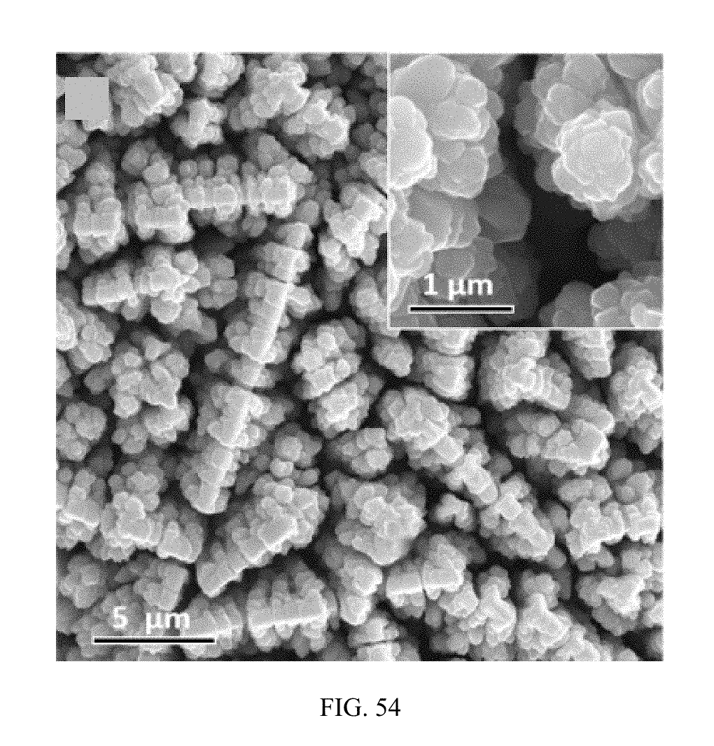

A 3-D hierarchically porous nickel framework (hp-Ni) electrocatalyst with 3D open porosity was prepared by a facile one-step self-template electrodeposition of metallic Ni framework on commercial Ni foam at about −3.0 A cm⁻² for about 500 s. The concomitant formation of H₂ bubbles during electrodeposition functioned as templates for the resulting porosity. The successful formation of metallic Ni for hp-Ni was verified by the corresponding X-ray diffraction (XRD) pattern (FIG. 52). Scanning electron microscopy (SEM) image (FIG. 53) indicated the 3D hierarchically macroporous feature of hp-Ni with pore sizes of several hundred micrometers, inherited from the pristine Ni foam. In addition, there were abundant smaller macropores with diameters of about 10 μm on the interconnected macropore walls of hp-Ni (FIG. 53), distinctively different from the featureless surface of a pristine Ni foam. A closer inspection of the smaller macropores at higher magnification showed 3D porous structure and the presence of stacked Ni nanospheres with smooth surface (FIG. 54). These distinct differences clearly confirmed the successful electrodeposition of porous Ni microspheres on Ni foam. Elemental mapping images revealed the presence of abundant Ni, plus a small amount of O due to slight surface oxidation in air. XPS analysis further confirmed the presence of metallic Ni and O, in line with the XRD and elemental mapping results. This hierarchically porous structure with 3D configuration may facilitate substrate transport and gas diffusion to boost the utilization efficiency of active sites, which are beneficial for electrocatalytic applications, among others.

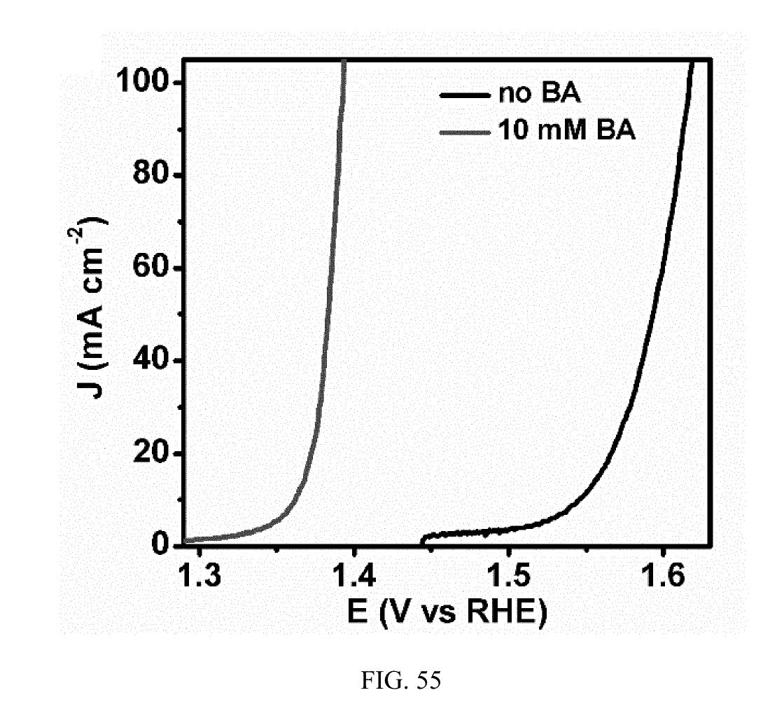

Alcohol oxidation is an important reaction in the chemical industry with applications ranging from petroleum chemical refining and biomass utilization to pharmaceutical and fine chemical synthesis, for example. The conventional methods for alcohol oxidation typically require stoichiometric chemical oxidants and expensive metal catalysts (such as Au, Pd, and Pt) under harsh conditions (e.g., high pressure and/or elevated temperature), hence it is of paramount importance to explore greener and lower-cost alternative methods for alcohol oxidation. Electrocatalytic oxidation under ambient conditions is an appealing approach as the oxidation can be solely performed by electricity without additional chemical oxidants. In this example, benzyl alcohol (BA) was chosen to evaluate the electrocatalytic activity of hp-Ni for alcohol oxidation, although other choices will be readily apparent to one of skill in the art. The target product is benzoic acid. Under alkaline condition (about 1.0 M KOH), the oxygen evolution reaction can potentially compete with organic oxidation reactions. Therefore, an ideal electrocatalyst may possess high preference towards BA oxidation rather than oxygen evolution, which guarantees the minimum Faradaic efficiency loss due to water oxidation. FIG. 55 shows linear sweep voltammetry (LSV) curves of BA oxidation and water oxidation catalyzed by hp-Ni in about 1.0 M KOH in the presence and absence of about 10 mM BA, respectively. In the absence of BA, the onset potential of hp-Ni was about 1.51 V vs. RHE and high current density beyond about 1.55 V vs. RHE was observed, implying the OER activity of hp-Ni. After adding about 10 mM BA, the hp-Ni exhibited negatively-shifted onset potential (about 1.35 V vs. RHE) and a large current density within about 1.40 V vs. RHE, indicating that the oxidation of BA on hp-Ni was significantly easier than OER. In stark contrast, the pristine nickel foam (NF) exhibited much worse activities for both OER and BA oxidation reaction, which may underscore the positive effect of 3D hierarchically porous Ni framework deposited on Ni foam for electrocatalysis. Such an improved catalytic performance of hp-Ni compared to that of NF is may be attributed to the synergetic effect of its unique 3D structure which facilitates gas diffusion and substrate transportation, together with higher electrochemically active surface area which will boost the utilization efficiency of active sites.

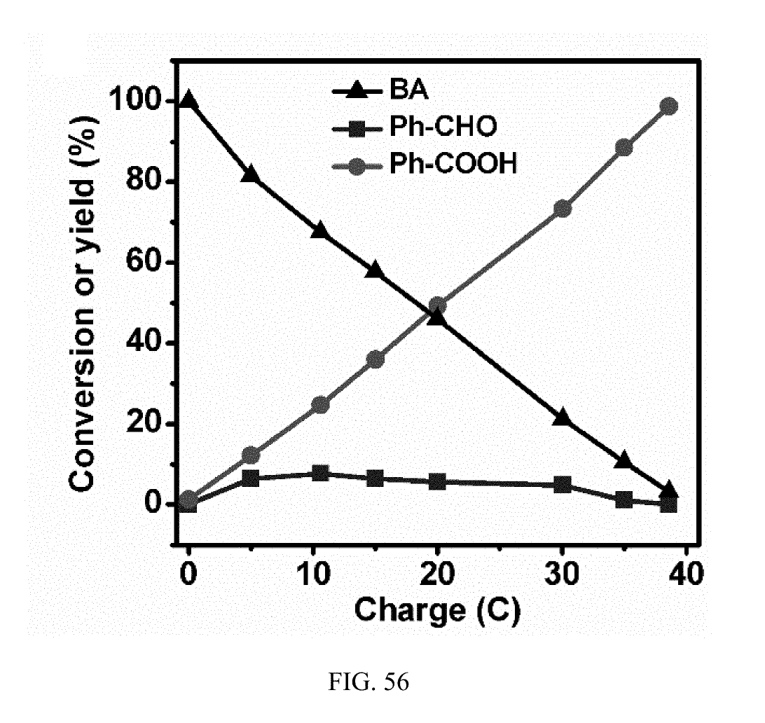

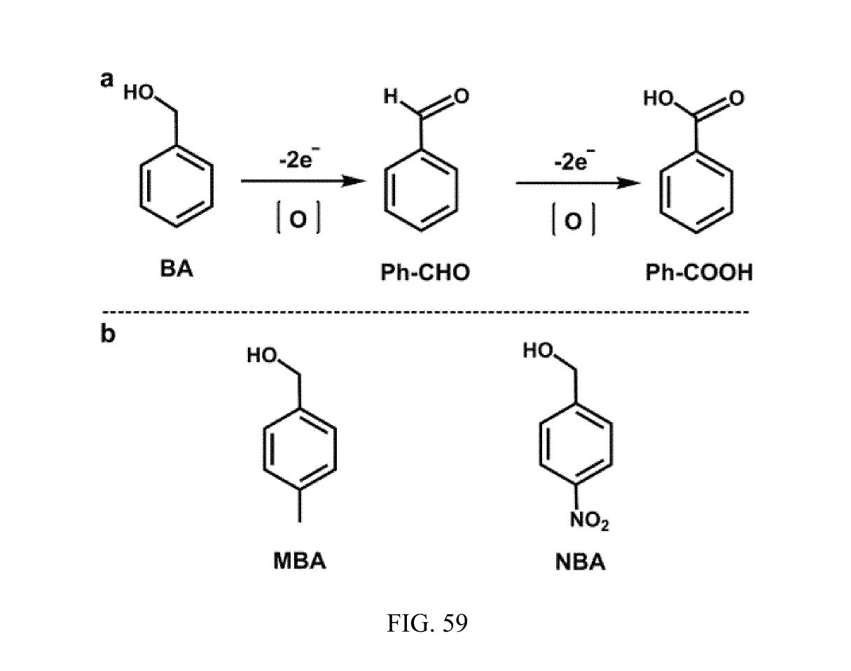

Next, long-term chronoamperometry utilizing hp-Ni as the electrocatalyst was carried out at about 1.423 V vs. RHE in about 10 mL, about 1.0 M KOH containing about 10 mM BA. The concentration evolution of BA and its oxidative products (benzyl aldehyde and benzoic acid) during the electrolysis were analyzed by HPLC. In this example, a theoretical charge of about 38 C was needed for the complete oxidation of BA to benzoic acid. As depicted in FIG. 55, no noticeable oxygen evolution could occur at about 1.423 V, hence a high Faradaic efficiency for benzyl alcohol (BA) oxidation may be anticipated. The resulting HPLC chromatograms demonstrated the increase of benzoic acid and decrease of BA over passed charges, implying the conversion of benzyl alcohol into benzoic acid (FIG. 56). After passing about 38 C, the peak of BA nearly disappeared while the benzoic acid peak reached its maximum intensity. Eventually, a Faradaic efficiency of about 98% was obtained. While the intermediate, benzaldehyde (FIG. 59a), was also detected during the chronoamperometry experiment, it remained at low concentration.

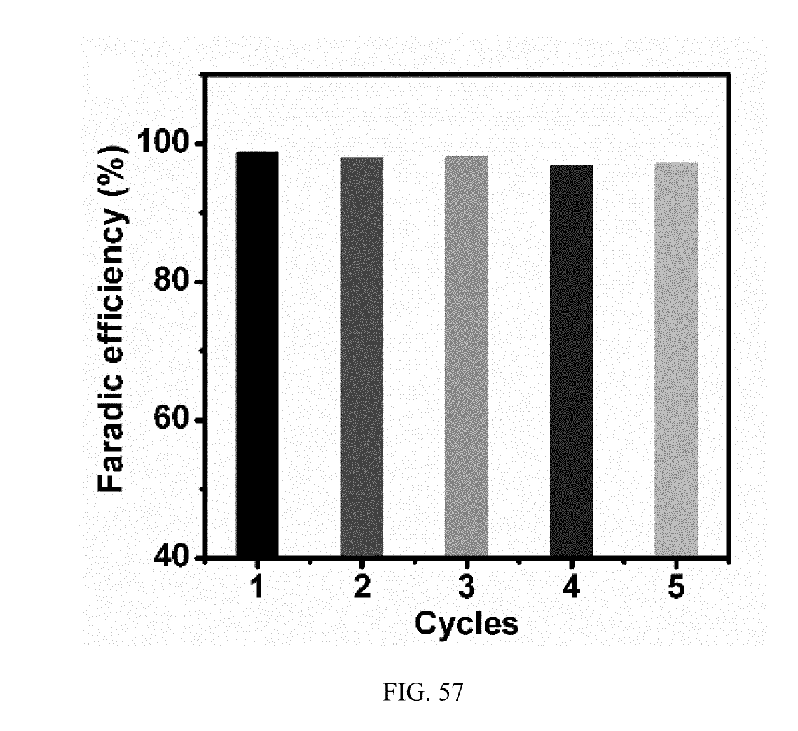

The stability of hp-Ni for BA oxidation was also estimated by repeating the above constant potential electrolysis using the same hp-Ni catalyst. As exhibited in FIG. 57, the Faradaic efficiencies of benzoic acid production for these five trials were calculated to be about 96-98%, demonstrating the robust durability of hp-Ni for BA oxidation.

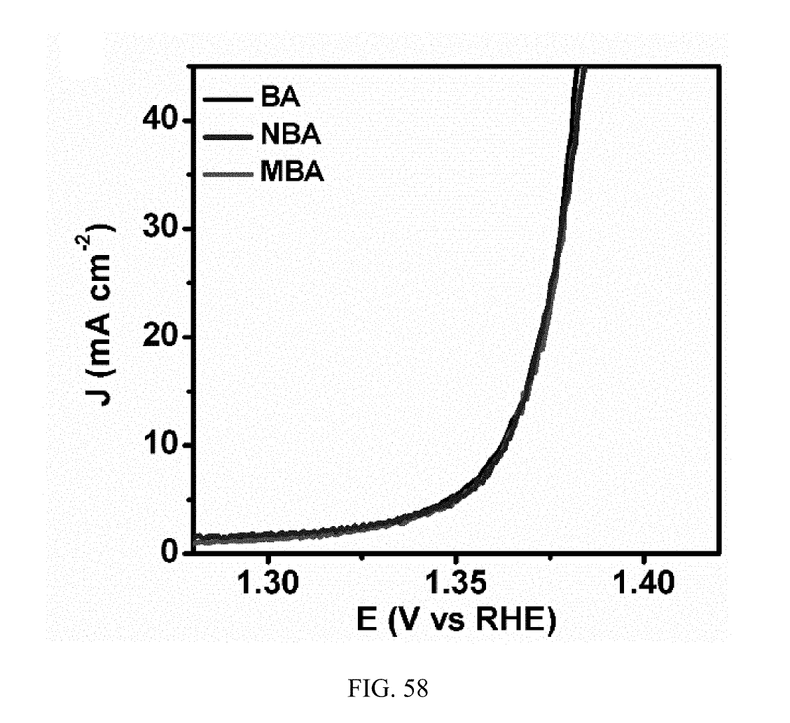

To investigate the electronic effect on the electrocatalytic activity of hp-Ni for alcohol oxidation, two derivatives of benzyl alcohol with electronic withdrawing (4-nitrobenzyl alcohol, NBA) and donating (4-methylbenzyl alcohol, MBA) substituents on the benzene ring (FIG. 59b) were subjected to similar electrocatalytic oxidation. As shown in FIG. 58, both LSV curves of these two new substrates took off at a quite similar potential compared to that of the parent benzyl alcohol. This result implies that the catalytic onset potential of these three alcohols oxidation may be primarily determined by the desirable oxidation state of the electrocatalyst, rather than their intrinsic thermodynamics, distinctive from many molecular electrocatalysts. Therefore, it may be that the optimal solid-state electrocatalysts for alcohol oxidation should require a lower potential to reach their functional oxidation states (high-valence states), which will be pursued in future studies.

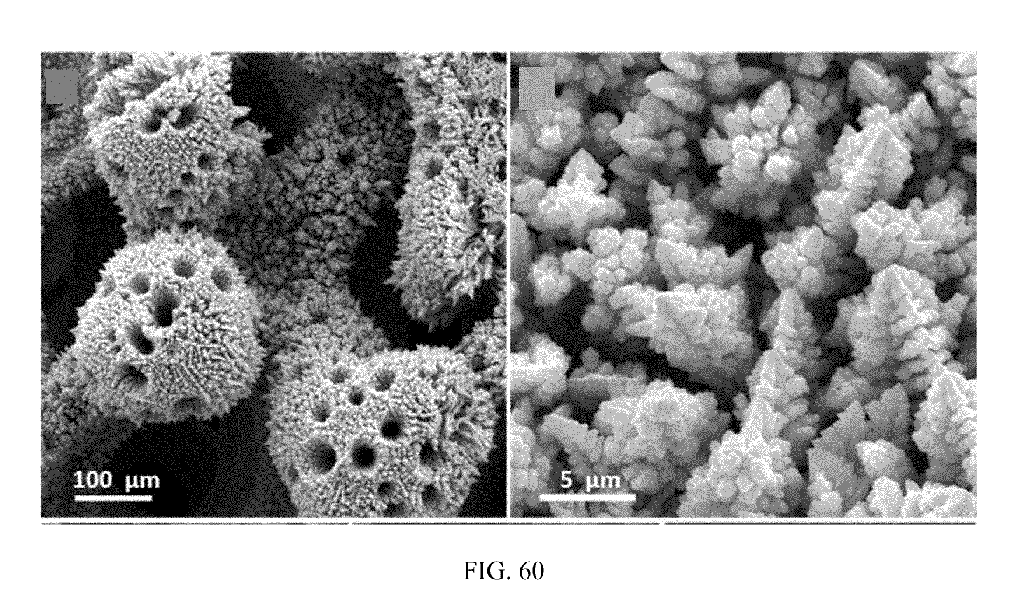

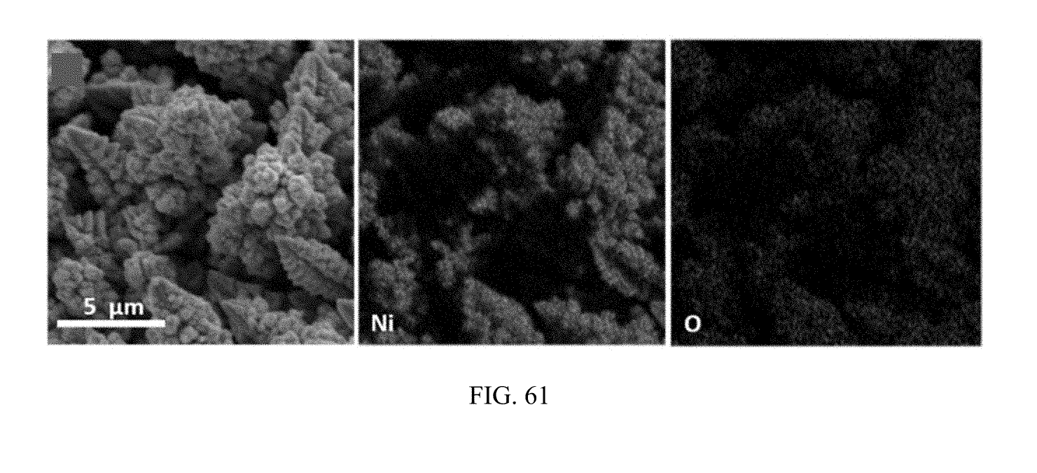

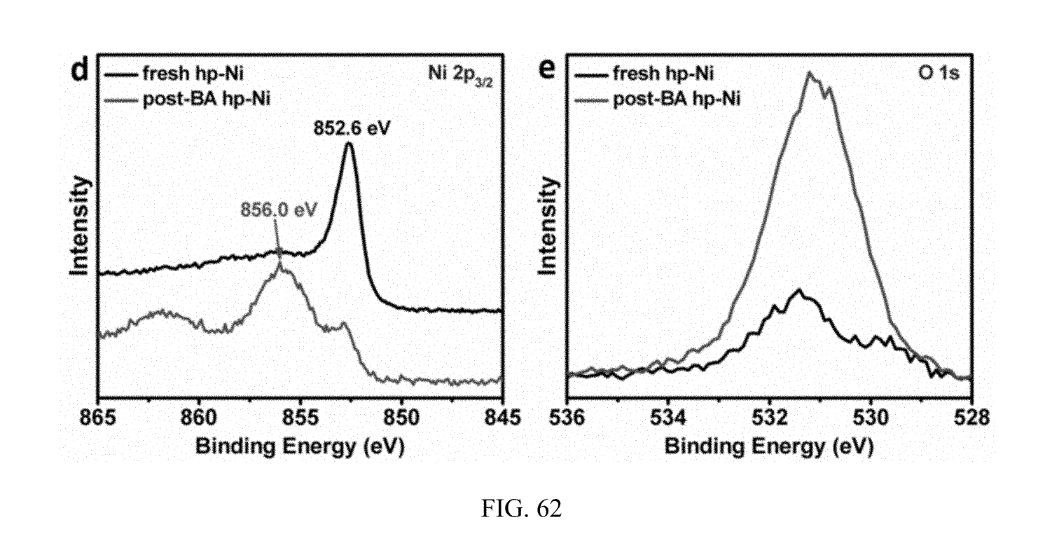

Then XRD, SEM, and XPS techniques were employed to probe the structure and composition details of the hp-Ni electrocatalyst after the above stability tests (named as post-BA hp-Ni). Although its SEM images at different magnifications (FIG. 60) and XRD pattern suggested the integrity of the whole three dimensional hierarchical porous morphology and mainly metallic Ni phase, elemental mapping results (FIG. 61) indicated that the presence of Ni and a large amount of O for post-BA hp-Ni. Energy-dispersive X-ray spectroscopy also revealed the increased O content in the post-BA hp-Ni relative to that of fresh hp-Ni, corroborating its surface oxidation during benzyl alcohol oxidation. Additionally, the high-resolution Ni 2p_{3/2} XPS spectra for the post-BA hp-Ni electrocatalyst showed a decreased peak at about 852.6 eV ascribed to metallic Ni, while an increased peak at about 856.0 eV attributed to oxidized Ni species, supporting the partial oxidation of metallic Ni (FIG. 62d). This nickel oxidation was further verified by the enhanced peak intensity of high-resolution O is XPS spectra (FIG. 62e). In view of the above, it may be that the catalytic active sites of benzyl alcohol and other related alcohols are the high-valent nickel species.

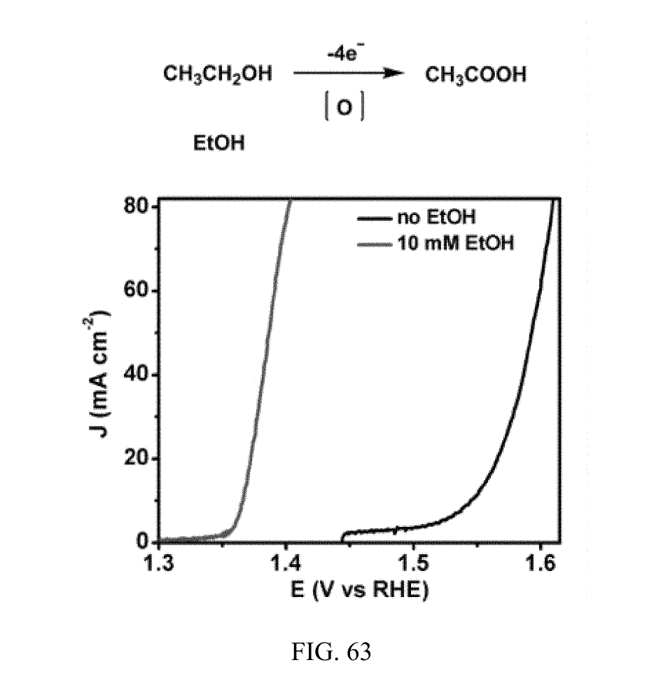

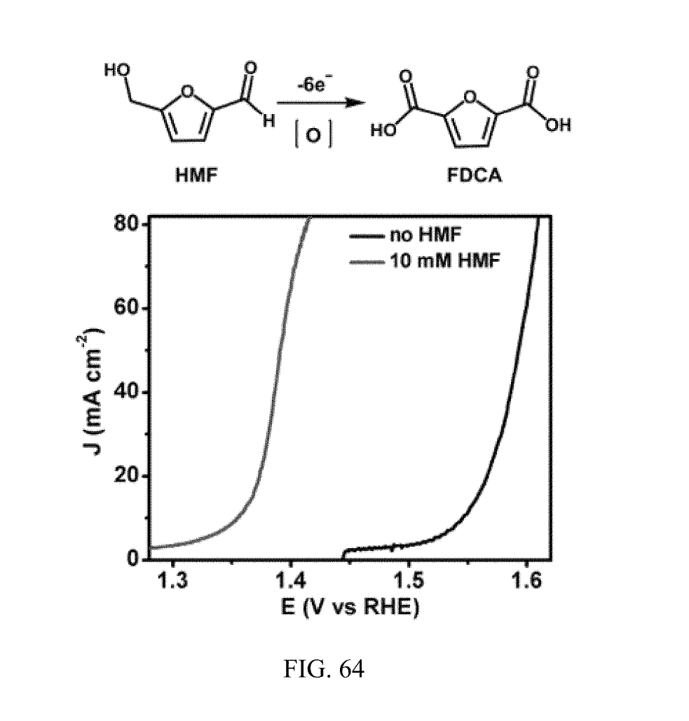

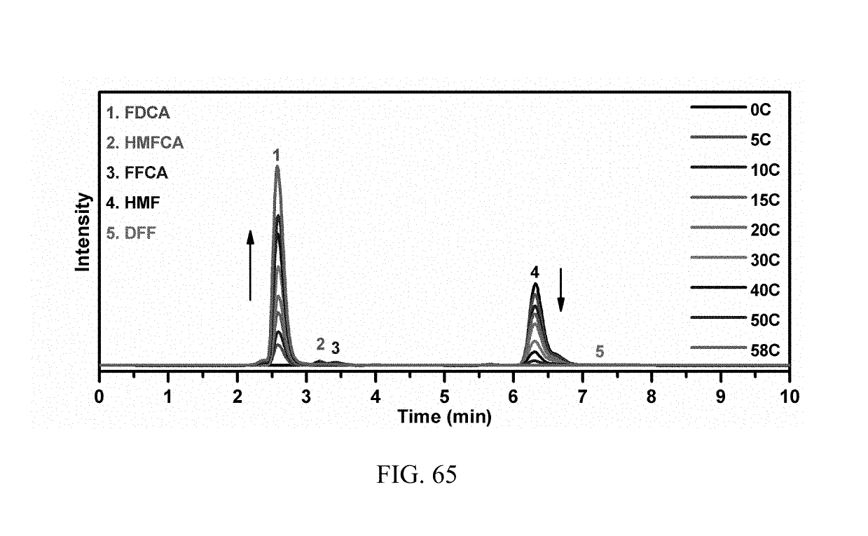

In order to demonstrate that such a strategy of HER coupled with alcohol oxidation is general and can be extended to upgrade other alcohol compounds, we further evaluated the performance of hp-Ni for the oxidative upgrading of ethanol and 5-hydroxymethylfurfural (HMF), both of which are known in the art as representative compounds for oxidative alcohol upgrading. As depicted in FIGS. 63 and 64, after adding about 10 mM ethanol and HMF, the catalytic onset potentials of hp-Ni both shifted to about 1.35 V vs. RHE and clear catalytic current density rises were viewed within about 1.40 V vs. RHE, indicative of more favorable alcohol oxidation than OER. The overpotentials to afford a benchmark current density (e.g., about 50 mA cm⁻²) for both ethanol and HMF oxidation reactions were at least about 200 mV smaller than that of OER (FIGS. 63 and 64). Chronoamperometry experiments carried out at about 1.423 V for the oxidation of ethanol and HMF further demonstrated the almost complete conversion to their corresponding value-added acid products (FIG. 63 top and FIG. 64 top). After passing the theoretical amount of charge, quantitative conversion to the desirable acids were obtained according to the corresponding ¹H NMR and HPLC results (FIG. 65).

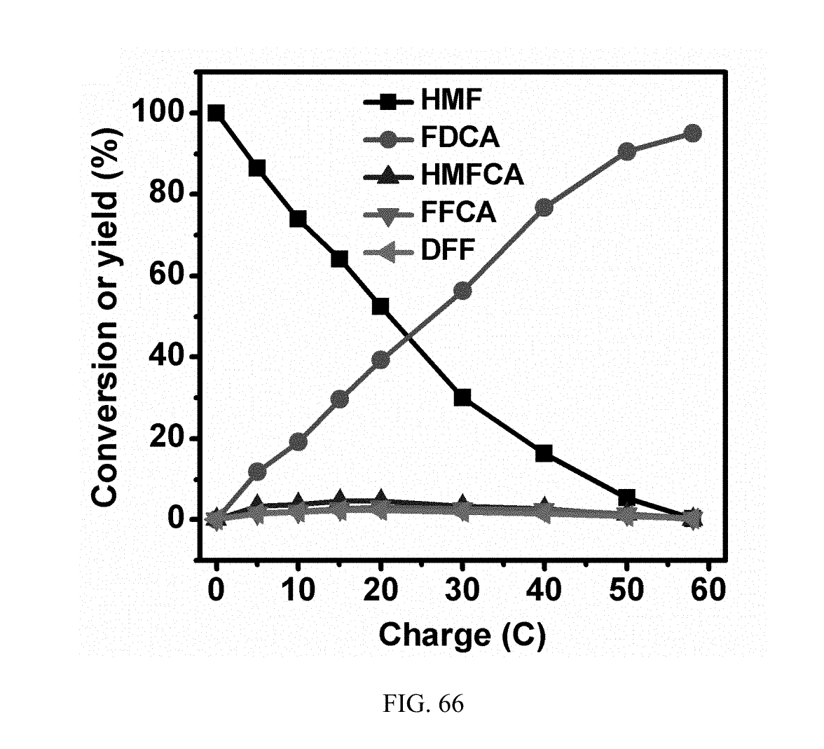

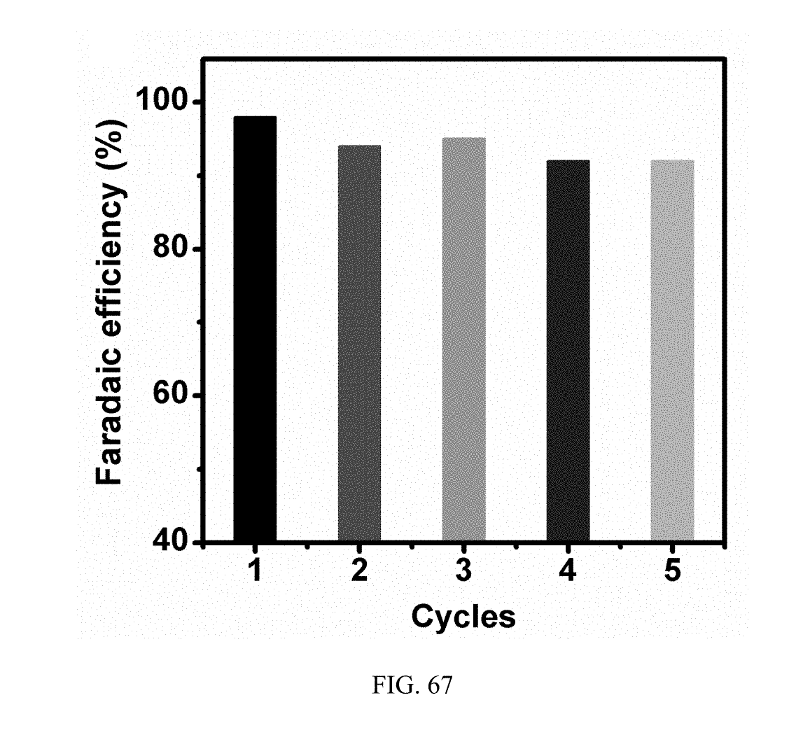

HMF, a dehydration product of C6 carbohydrates from biomass, is important in biomass refinery, as HMF is a platform chemical and can be upgraded to a wide variety of important commodity chemicals, including 2,5-bishydroxymethulfuran, 2,5-dimethylfuran, and 2,5-furandicarboxylic acid (FDCA). In particular, FDCA may be a substitute of terephthalic acid to produce polyamides, polyesters, and polyurethanes. Because of the role of HMF as a biomass-derived intermediate compound, the details of HMF oxidation to FDCA on hp-Ni was also examined through a similar chronoamperometry experiment at about 1.423 V vs. RHE in about 10 mL, about 1.0 M KOH with about 10 mM HMF. In this case, the theoretical amount of about 58 C charge was calculated for the complete oxidation of HMF to FDCA. As shown in FIG. 66, a high Faradaic efficiency for oxidation of HMF may also be expected due to the absence of water oxidation at about 1.423 V vs. RHE. The obtained HPLC chromatograms (FIG. 65) evidently exhibited the decrease of peak for HMF and rise of peak for FDCA over electrolysis time, intimating the conversion from HMF to FDCA (FIG. 66). After passing about 58 C, a Faradaic efficiency of about 98% was obtained for the FDCA production. There may be two possible paths for oxidation of HMF to FDCA: one starts from the oxidation of the aldehyde group of HMF to generate HMFCA and the other proceeds through the formation of DFF. Subsequently, both routes yield FFCA prior to the eventual formation of FDCA. The relatively higher concentration of HMFCA relative to that of DFF revealed the hp-Ni-mediated HMF oxidation most likely followed the first-step formation of HMFCA route, similar to those commonly observed aerobic oxidations. Nevertheless, the DFF route was also included, since the DFF intermediate was still identified via HPLC (FIG. 65). Moreover, the high Faradaic efficiencies (about 92-98%) of FDCA formation for five successive electrolysis cycles demonstrated the robust stability of hp-Ni for HMF oxidation as well (FIG. 67).

The hp-Ni exhibited great activity for HER. The LSV curve of hp-Ni showed a small onset potential and achieved a HER current density of about −50 mA cm⁻² at an overpotential of approximately about 219 mV in about 1.0 M KOH, comparable to or even better than those of known nonprecious HER catalysts like β-Mo₂C (greater than about 250 mV), η-MoCₓ/C (about 220 mV), CoP/CC (greater than about 300 mV), and other HER catalysts to reach the same current density. Moreover, hp-Ni exhibited robust long-term stability, as revealed by its stable overpotential of about 230 mV to reach about −50 mA cm⁻² for an about 18 h chronopotentiometry experiment.

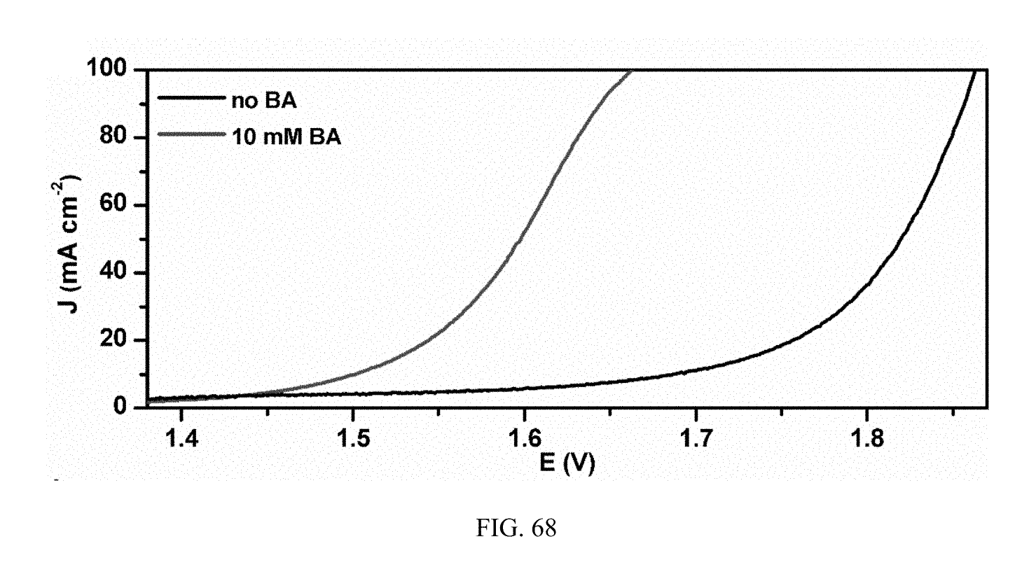

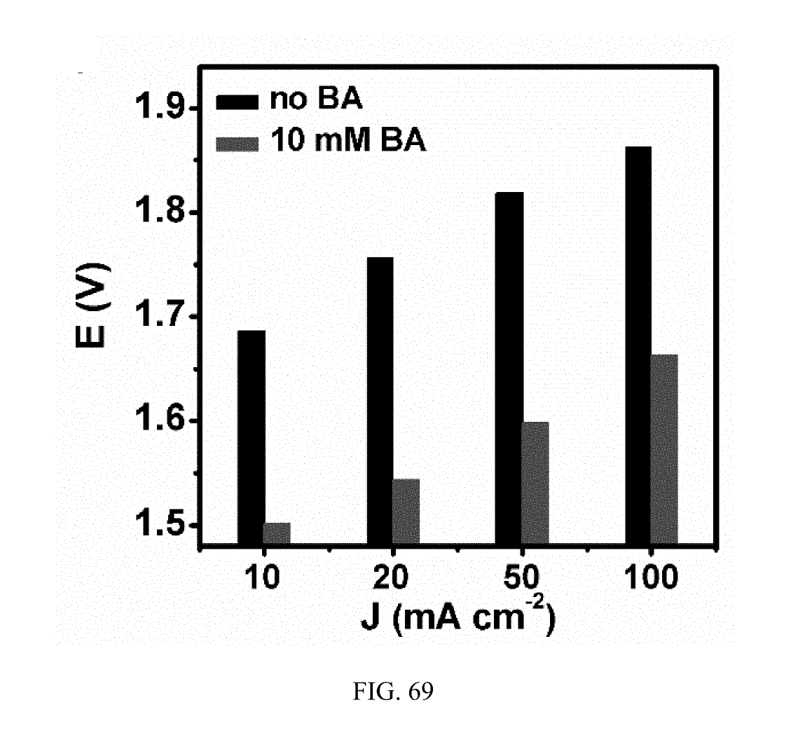

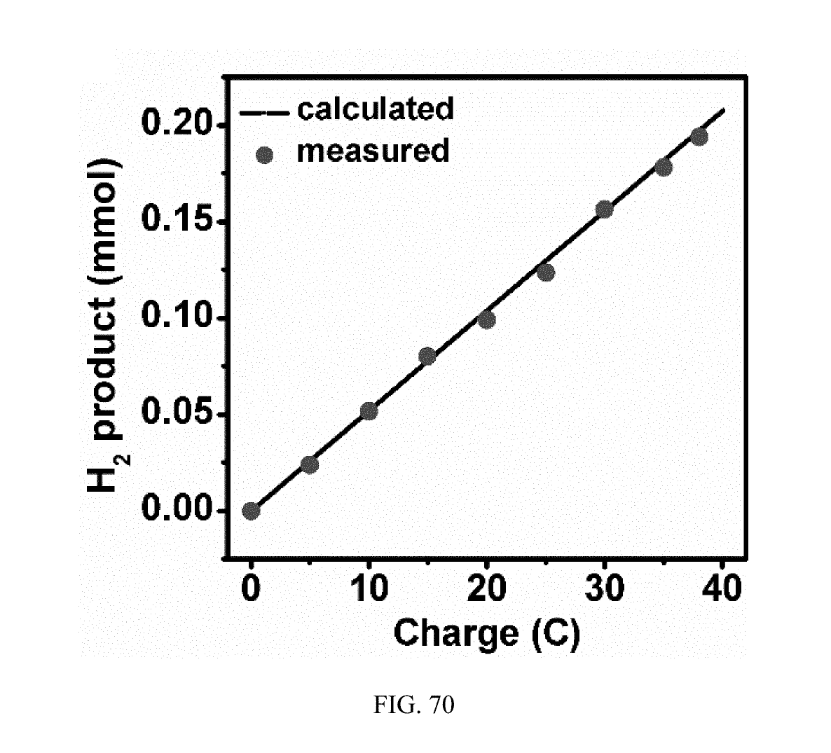

Given the markedly improved performance of hp-Ni for both alcohol oxidation and H₂ evolution as aforementioned, it is likely that hp-Ni can function as a bifunctional electrocatalyst for simultaneous production of H₂ and organic acid in a two-electrode configuration under alkaline condition. In order to validate this hypothesis, we chose benzyl alcohol as the organic substrate and an anion exchange membrane was used to separate the two electrodes. As depicted in FIG. 68, the hp-Ni catalyst couple needed a cell voltage of about 1.69 V to afford about 10 mA cm⁻² for water splitting, comparable to or even better than those of known noble metal-free bifunctional water splitting catalysts, including NiFe LDH/NF (about 1.70 V), Ni₅P₄iNF (about 1.70 V), Ni₃S₂/NF (greater than about 1.70V), and others to reach same current density. Perhaps more importantly, upon addition of about 10 mM benzyl alcohol, the cell voltages to afford about 10, about 20, about 50, and about 100 mA cm⁻² were markedly reduced to about 1.50, about 1.54, about 1.60, and about 1.66 V, respectively (FIG. 69), substantially smaller than those of pure water electrolysis. In order to quantify the generated H₂ and benzoic acid under this two-electrode setup, a durable electrolysis was performed at a cell voltage of about 1.50 V. As exhibited in FIG. 70, the produced H₂ measured by gas chromatography (GC) agreed with the theoretical amounts very well, suggesting a high Faradaic efficiency of about 100% for HER. Quantifying the resulting liquid products by HPLC also implied a Faradaic efficiency of about 97% for benzoic acid formation. If HMF was selected as the organic substrate, similar low energy input and high Faradaic efficiencies for the production of both H₂ and FDCA was obtained, demonstrating the versatility of this new-type electrolysis.

Described in this example is a general strategy for concurrent H₂ generation and alcohol oxidation catalyzed by a low-cost hp-Ni with nearly unity Faradaic efficiencies. Possibly owing to the more favourable thermodynamics of these alcohol oxidations than that of OER on hp-Ni, the electrolyzer voltage to produce benchmark current densities was reduced by about 220 mV compared to water splitting electrolysis. Additionally, value-added products were generated at both electrodes (H₂ at cathode and valuable organic acids at anode). This strategy provides an alternative approach to avoid the issues of H₂/O₂ mixing and ROS formation during traditional water electrolysis. Given the advantages of inexpensive catalyst, high energy conversion efficiency, great Faradaic efficiency, and ambient reaction condition (room temperature, atmospheric pressure, and aqueous solution), this new-type electrolysis strategy of cathodic H₂ production coupled with anodic alcohol oxidation may apply to many other oxidative organic upgrading reactions to pair with HER, maximizing energy conversion efficiency and yielding valuable products. Finally, the similar onset potential of hp-Ni for those diverse alcohol substrates with different intrinsic oxidation thermodynamics may imply that the catalytic onset is largely determined by the desirable oxidation potential of hp-Ni. Hence, rational design of catalysts requiring lower oxidation potential is anticipated to lead to electrocatalytic organic oxidation at even smaller voltage input. Further studies along this line are underway.

Chemicals: Benzyl alcohol (BA), benzoic acid, benzaldehyde, 4-nitrobenzyl alcohol (NBA), and 4-methylbenzyl alcohol (MBA) were used as received from a commercial source. Ethanol was used as received from a commercial source. 5-Hydroxymethylfurfural (HMF) and 2,5-furandicarboxylic acid (FDCA) were purchased from commercial sources. 2,5-diformylfuran (DFF) and 2-formyl-5-furancarboxylic acid (FFCA) were purchased from a commercial source. 5-Hydroxymethyl-2-furan-carboxylic acid (HMFCA) was purchased from a commercial source. Potassium hydroxide was used as received from a commercial source. Ammonium chloride was purchased from a commercial source. Nickel chloride was purchased from a commercial source. Nickel foam with purity greater than about 99.99% was purchased from a commercial source. The anion exchange membrane (Fumasep FAA-3-PK-130) was purchased from a commercial source. All chemicals in this example were used as received without purification. Water deionized (about 18 MΩ·cm) from a Barnstead E-Pure system was used in all experiments described in this example.

Synthesis of hp-Ni electrocatalyst: The hp-Ni bifunctional electrocatalyst was prepared by a facile template-free cathodic electrodeposition of 3D hierarchically porous Ni microspheres on a nickel foam (hp-Ni). Typically, the electrodeposition was performed under a standard two-electrode configuration at about room temperature with an electrolyte consisting of about 2.0 M NH₄Cl and about 0.1 M NiCl₂. A piece of commercial nickel foam with a size of about 0.5 cm×about 0.5 cm was employed as the working electrode and a Pt wire as the auxiliary electrode. The galvanostatic electrodeposition was carried out at about −3.0 A cm⁻² for about 500 s to obtain hp-Ni samples with a mass loading of about 75 mg cm⁻². Many variations on these procedures will be readily apparent to one of skill in the art.

Physical methods: XPS data energy corrections on high resolution scans were calibrated by referencing the C is peak of adventitious carbon to about 284.5 eV. Other experimental details, such as specific commercial or government instruments, software, and facilities commonly used to conduct these physical methods, are known to those in the art.

Electrocatalytic measurements: Electrochemical HER, OER, and alcohol oxidation measurements were performed under three-electrode configuration. The as-prepared hp-Ni was directly used as working electrode, a Ag/AgCl (sat. KCl ) electrode as reference electrode, and a carbon rod as counter electrode. All the reported potentials in this example were quoted with respect to reversible hydrogen electrode (RHE) through E_{RHE}=E_{Ag/AgCl}+0.059×pH+0.197 V, and overpotential for OER (η) was calculated from η=E_{RHE}-1.23 V. The electrochemical HER, OER, and alcohols oxidation experiments were conducted in about 10 mL, about 1.0 M KOH solution in the presence or absence of about 10 mM organic substrates. H₂ and O₂-saturated electrolytes were used for HER and OER measurements, respectively. For two-electrode electrolysis, the two hp-Ni were employed as bifunctional catalyst electrodes for both anode and cathode. All the potential range in this example was scanned at a scan rate of about 2 mV s⁻¹. iR (current times internal resistance) compensation was employed in all the electrochemical measurements in this example to adjust the voltage drop between reference and working electrodes. The stability tests of hp-Ni for BA and HMF oxidation were evaluated by chronoamperometry at about 1.423 V vs. RHE in about 10 μL, about 1.0 M KOH containing about 10 mM corresponding organic substrates for five consecutive cycles.

Product quantification. To analyse the products of BA and HMF oxidation quantitatively and calculate the corresponding Faradaic efficiencies, about 10 μL aliquots of the electrolyte solution during chronoamperometry testing were collected periodically from the electrolysis solution and diluted with about 490 μL water, which were then analysed using HPLC. The wavelengths of detector were set at about 230 nm for benzoic acid, about 254 nm for benzyl alcohol and benzaldehyde, and about 265 nm for HMF and its corresponding products. An eluent mixture of about 5 mM ammonium formate aqueous solution (A) and methanol (B) was used. The HPLC analysis was conducted for the BA oxidation products by A/B (v/v: 4/6), while HMF oxidation products by A/B (v/v: 7/3) within about 10 min at a flow rate of about 0.5 mL min⁻¹. The qualitative and quantitative analyses of reactants and products were conducted based on the corresponding calibration curves by applying standard solutions with known concentrations.

Gas chromatography with a Molecular Sieve 13 packed column, a HayesSep D packed column, and a thermal conductivity detector was used to quantify the generated H₂ during electrolysis. The oven temperature was set at about 80° C. and argon was used as the carrier gas.

### Example 4

### Integrating Electrocatalytic 5-hydroxymethylfurfural Oxidation and Hydrogen Production via Co—P-Derived Electrocatalysts

To develop competent and low-cost electrocatalysts, one may integrate oxidative biomass upgrading with H₂ production in a single electrolyzer. In this example, it is reported that electrodeposited Co—P can be directly utilized as the electrocatalyst for the conversion of HMF to FDCA in alkaline solution. A nearly complete HMF conversion and about 90% yield of FDCA were obtained in about 1.0 M KOH under ambient condition (about 1 atm and about room temperature). Simultaneously, Co—P was able to catalyze H₂ evolution as the cathode reaction with about 100% Faradaic efficiency as well.

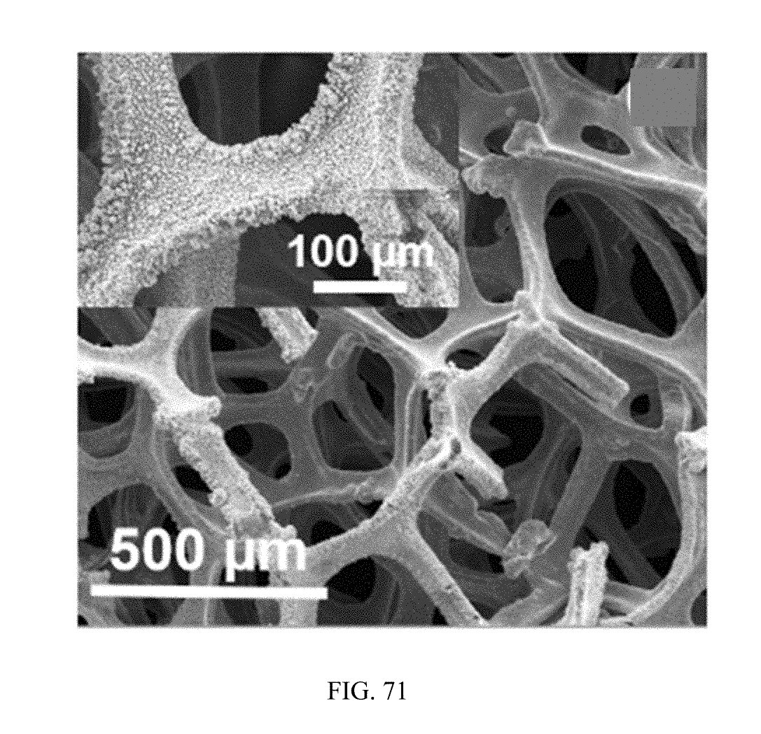

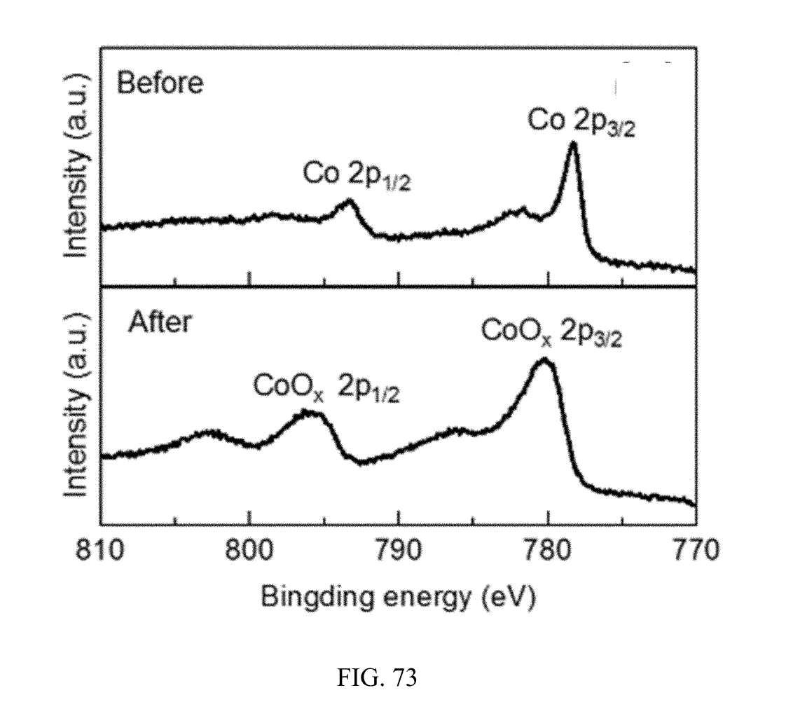

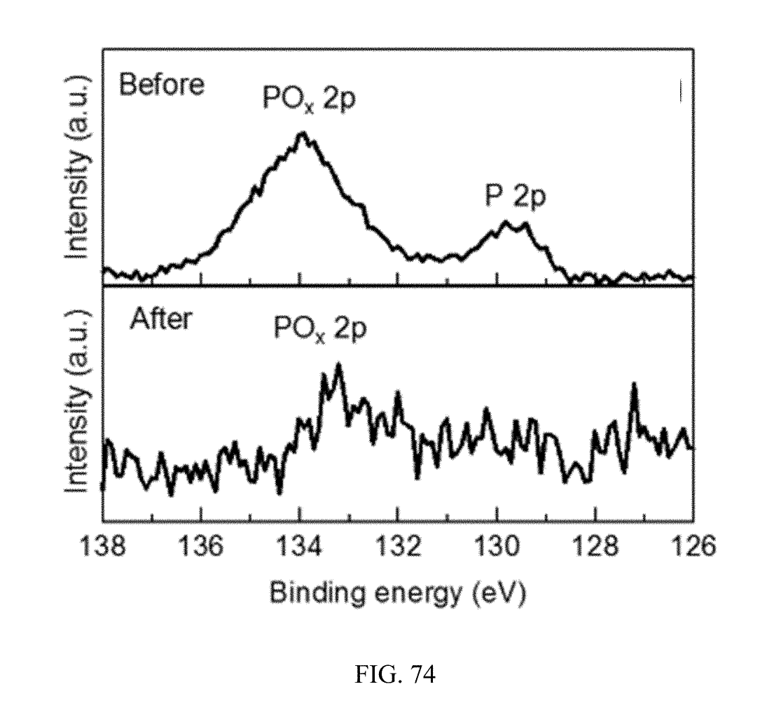

The preparation of phosphorous-doped cobalt (10% P in 90% Co) (Co—P) on copper foam (Co—P/CF) was conducted. FIG. 71 displays the scanning electron microscopy (SEM) images of the as-prepared Co—P/CF, showing a nearly complete coverage of the copper foam by Co—P particles (FIG. 71 inset). The elemental mapping images confirmed the presence of cobalt and phosphorous and their even distribution in Co—P. X-ray photoelectron spectroscopy (XPS) was carried out to probe the identity and valence state of each element in the electrodeposited catalyst. The XPS survey spectrum was generated of the as-prepared Co—P/CF, showing all the anticipated elements, including cobalt, phosphorous, and copper (from the substrate). The high-resolution Co 2p XPS spectrum (FIG. 73 top) displayed two peaks at about 778.3 and about 793.4 eV, corresponding to the Co 2p_{3/2} and 2p_{1/2} binding energies, respectively. These values are quite close to those of metallic cobalt. The high-resolution P 2p spectrum (FIG. 74 top) exhibited a dominant feature at about 133.9 eV, which may be attributable to oxidized phosphorous species due to the oxidation in air. The other peak at about 129.4 eV was assigned to the anticipated phosphide signal. The X-ray diffraction (XRD) pattern of Co—P/CF only exhibited the crystalline peaks of the copper foam plus Cu₂O, implying the amorphous nature or very small crystal size of the deposited Co—P.

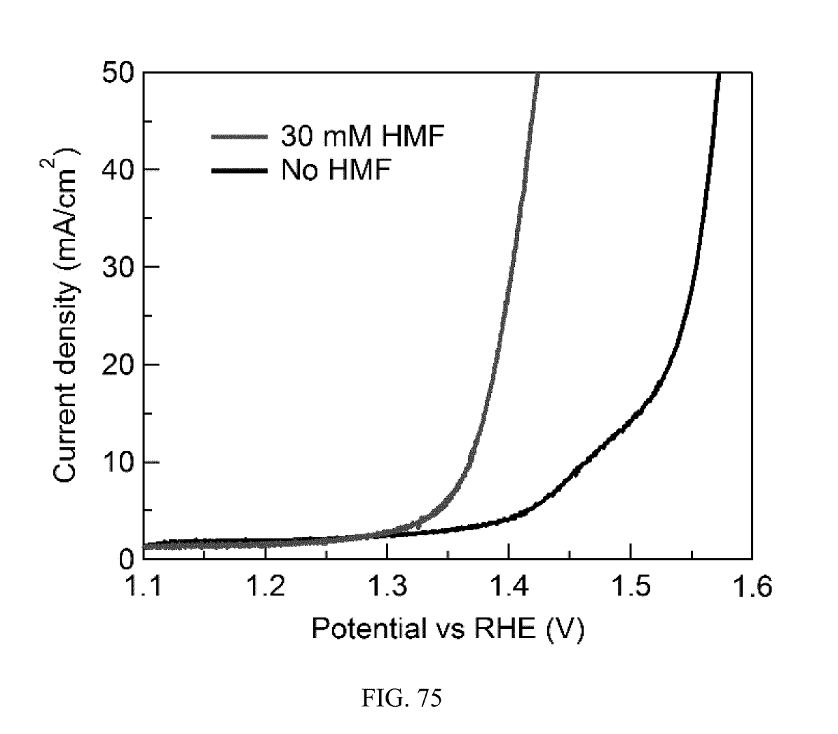

Co—P is quite active for H₂ evolution under strongly alkaline conditions, and described in this example is an evaluation of its catalytic performance for HMF oxidation in about 1.0 M KOH. All the following electrochemistry experiments in this example were conducted in a three-electrode configuration with a two-compartment cell unless otherwise noted. The most common competing reaction for HMF oxidation in aqueous media is typically water oxidation to O₂. As shown in FIG. 75, the linear sweep voltammetry (LSV) of Co—P/CF in the absence of HMF exhibited an anodic catalytic current beyond about 1.5 V vs RHE (reversible hydrogen electrode) accompanied with vigorous bubble release upon further positive scan. This catalytic current was confirmed as water oxidation to O₂. When about 30 mM HMF was added, the catalytic current onset shifted cathodically to about 1.30 V vs RHE, which implied that Co—P/CF was able to preferably catalyze HMF oxidation at potentials less positive than those required for water oxidation in alkaline media. The catalytic current increased dramatically beyond about 1.35 V and reached a catalytic current density of about 20 mA/cm² at about 1.38 V vs RHE, about 150 mV smaller than that of water oxidation (about 1.53 V vs RHE to reach about 20 mA/cm²). The blank copper foam was less active than Co—P/CF for HMF oxidation.

Scheme 3 presents two possible pathways for the oxidation of HMF to FDCA: (i) the hydroxymethyl group of HMF is first oxidized to form 2,5-diformylfuran (DFF) and subsequently its two aldehyde groups are oxidized consecutively to yield 5-formyl-2-furancarboxylic acid (FFCA) and then FDCA; (ii) the aldehyde group of HMF is first oxidized to form 5-hydroxymethyl-2-furancarboxylic acid (HMFCA), followed by the oxidation of the hydroxymethyl group to form FFCA and later FDCA. Both pathways converge at the same intermediate FFCA prior to the final product FDCA.

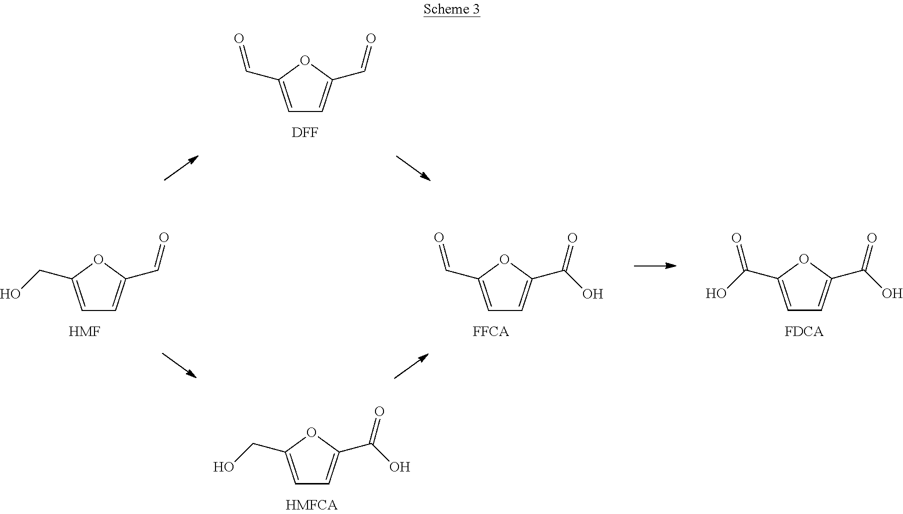

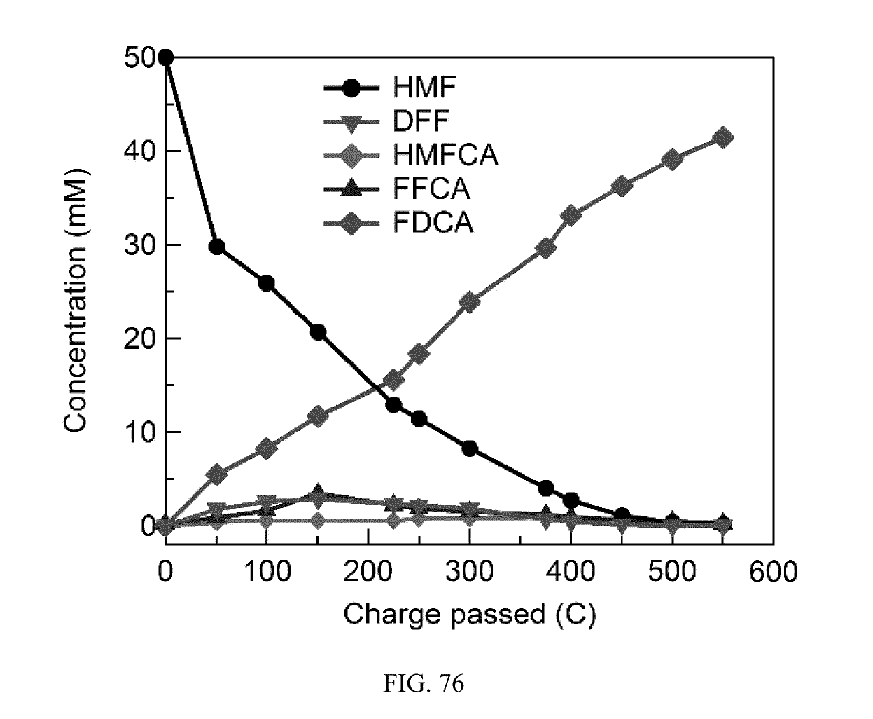

To investigate the conversion of HMF and the yields of oxidation products over time, an about 6-h controlled potential electrolysis was conducted for about 50 mM HMF in about 1.0 M KOH with an applied potential of about 1.423 V vs RHE. As shown in FIG. 75, water oxidation could not occur at this potential. Since the oxidation of HMF into FDCA is a six-electron process, it was calculated that about 560 C was required to completely convert about 50 mM HMF if the FDCA yield was 100%. The concentration change of HMF and its corresponding oxidation products were quantified with high-performance liquid chromatography (HPLC) via analyzing aliquots periodically collected from the electrolyte. FIG. 76 presents the concentration change of HMF and its oxidation intermediates/products over the about 6-h electrolysis. The conversion rate of HMF was quite fast during the first hour of electrolysis and slowed down later on, which was consistent with the decreased concentration of HMF over time. Different from TEMPO-mediated electrocatalytic HMF oxidation, wherein the intermediate FFCA was accumulated during the first half of electrolysis and the subsequent FDCA formation started after the concentration of FFCA reached its maximum, in this system FDCA was yielded right after the commencement of electrolysis. The HPLC traces of those electrolyte aliquots were collected when the consumed charge was about 0, about 100, about 200, about 300, about 400, and about 560 C. All the three intermediates, DFF, HMFCA, and FFCA, remained at low concentration throughout the entire electrolysis. In fact, FDCA exhibited as the only primary product of HMF oxidation catalyzed by Co—P/CF in about 1.0 M KOH. As shown in FIG. 76, after about 560 C charge was consumed, nearly about 90% yield of FDCA were obtained. The carbon balance of HMF oxidation was displayed. After electrolysis, acidification of the resulting electrolyte solution with H₂SO₄ to pH about equal 0 enabled the final product to be isolated as a white precipitate. The proton nuclear magnetic resonance spectrum of the precipitate in D₂O further confirmed the identity of the final product as FDCA; while the initial electrolyte only contained HMF.

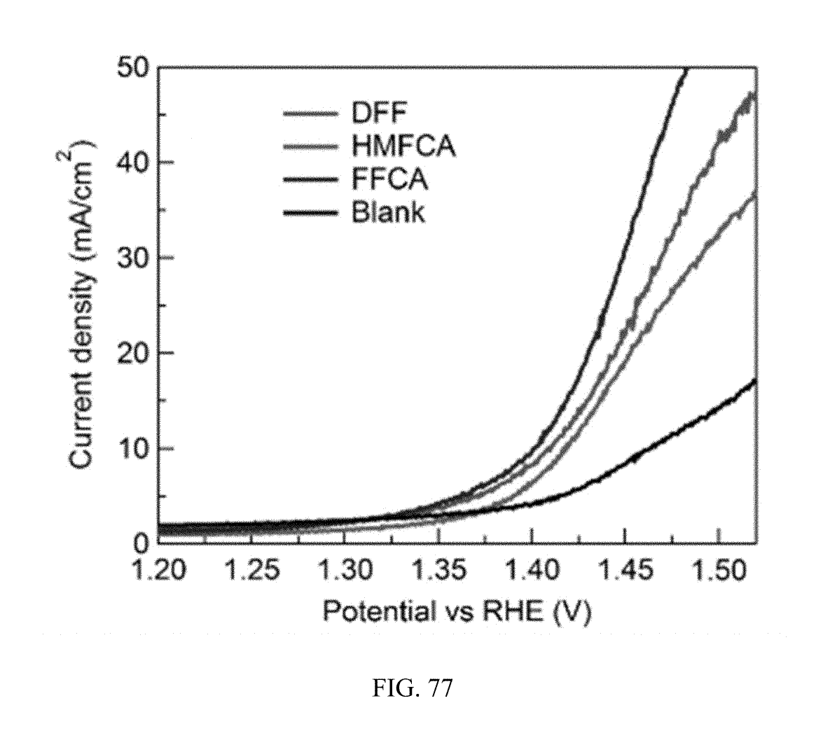

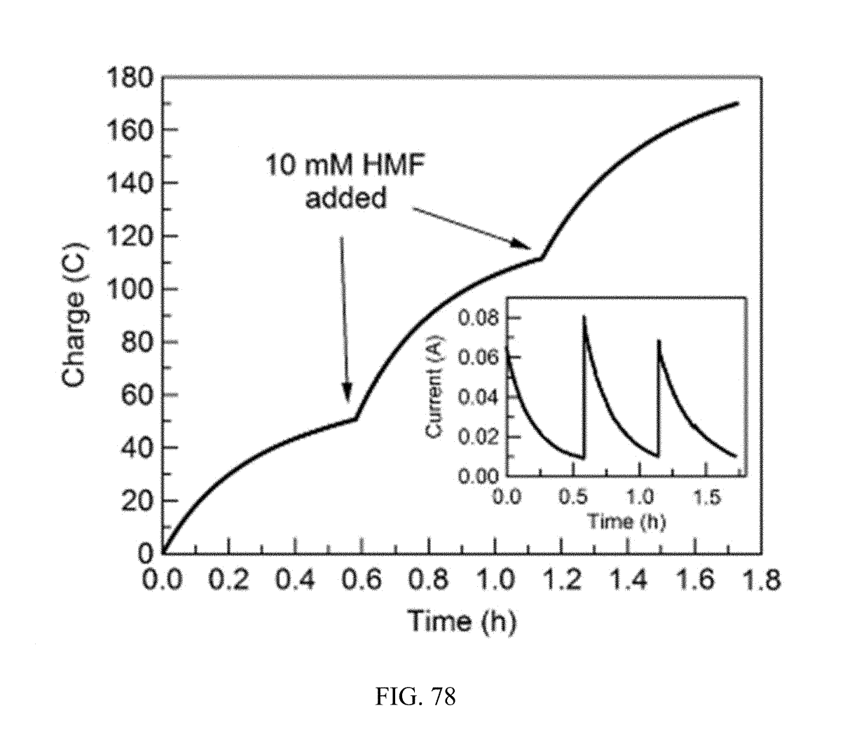

Since negligible HMFCA was detected during the HMF oxidation catalyzed by Co-P/CF (FIG. 76), it might be assigned that the Co—P-catalyzed HMF oxidation followed the DFF pathway. The DFF pathway was proposed for the TEMPO-mediated electrocatalytic HMF oxidation, however most aerobic oxidation reactions typically proceed through HMFCA as the dominant pathway. Since the FFCA concentration remained low during the conversion process, it may be that the oxidation rate of FFCA must be faster than or comparable to its formation rate (i.e., the conversion rate of DFF). FIGS. 77 and 78 plot the LSV curves of Co—P/CF in the presence of about 30 mM DFF, HMFCA, or FFCA. The LSV curve collected in the absence of any organic substrates was also included for comparison. Even though these three substrates could be preferentially oxidized by Co—P/CF relative to water oxidation, it was apparent that Co-P exhibited different activities towards their oxidation. Based on the catalytic onset and current density, the activity order was FFCA>DFF>HMFCA. Therefore, as FFCA could be oxidized at a rate faster than that of DFF, we understood that the concentration of FFCA remained low during the entire HMF oxidation process.

In order to evaluate the robustness of Co—P/CF for HMF oxidation, a three-cycle electrolysis experiment was performed at about 1.423 V vs RHE starting with about 10 mM HMF (FIG. 78). After about half an hour, the catalytic current decreased to background capacitance current due to the nearly complete consumption of HMF (FIG. 78 inset). However, upon addition of another about 10 mM HMF, the catalytic current resumed, implying the excellent stability of Co—P/CF for electrocatalytic HMF oxidation. Such a catalytic activity resumption could be further confirmed from the third addition of about 10 mM HMF (FIG. 78).

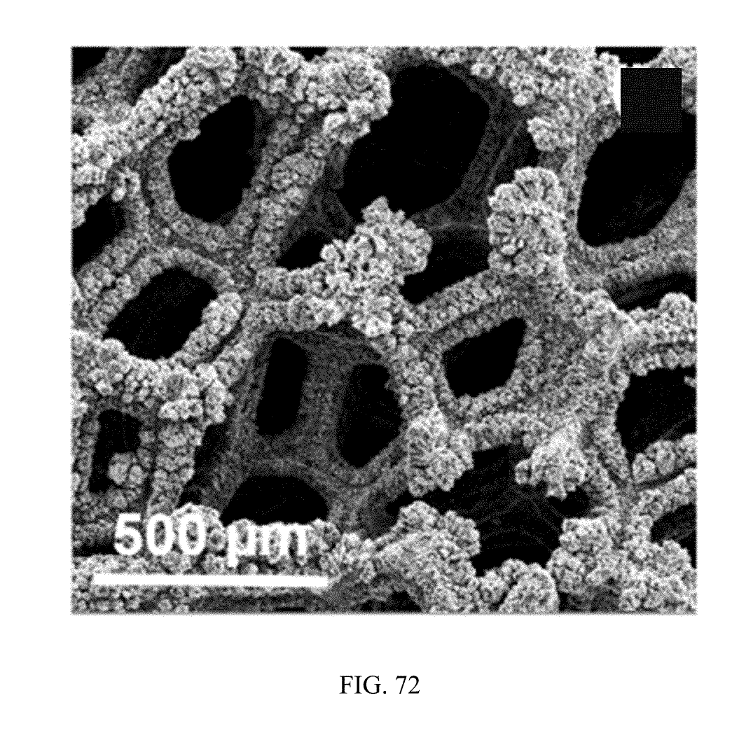

Post-electrolysis analysis was also conducted on the Co—P/CF after the chronoamperometry experiment at about 1.423 V for about 6 h. In contrast to the as-prepared sample (FIG. 71), post-electrolysis Co—P/CF contained large aggregates and nanoparticles on the copper foam (FIG. 72). Nevertheless, elemental mapping analysis indicated the even distribution of cobalt, albeit the phosphorous amount was largely diminished. Similar to the reported Co—P after a long-term electrolysis for O₂ evolution, it may be that both cobalt and phosphorous would be oxidized on the catalyst surface and a large amount of oxygen would be observed. Indeed, the high-resolution Co 2p XPS spectrum of the post-HMF-oxidation Co—P/CF displayed two peaks at about 780.7 and about 796.3 eV (FIG. 73 bottom), which could be attributed to cobalt oxides/hydroxides. Similarly, a peak at about 133.9 eV in the high-resolution P 2p XPS spectrum could be assigned to oxidized phosphorous species (FIG. 74 bottom). No apparent crystalline peaks of CoOₓ were detected in its XRD pattern, suggesting an amorphous nature.

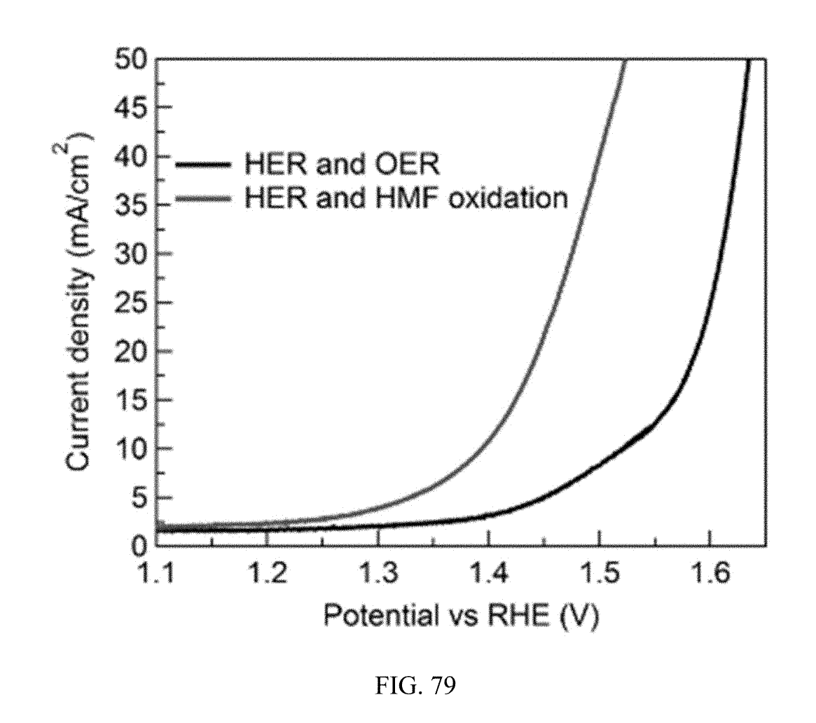

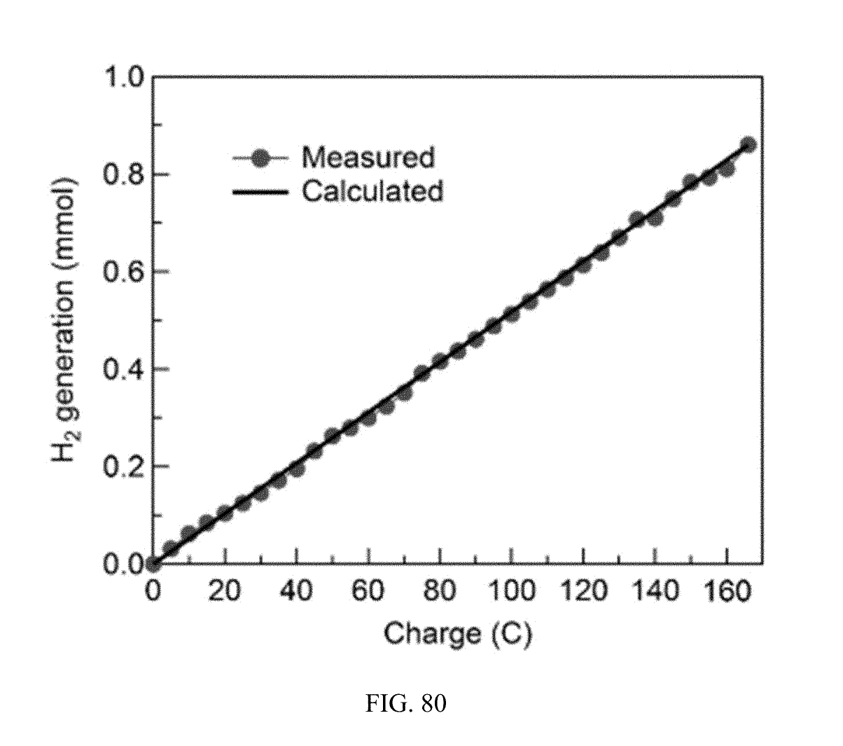

Based on the HMF oxidation results aforementioned, an electrochemical cell in two-electrode configuration was constructed with HMF oxidation as the anode reaction and H₂ production as the cathode reaction. In this electrolyzer, Co—P/CF was utilized as the catalysts for both anode and cathode (Co—P/Co—P catalyst couple). The linear sweep voltammogram of the integrated HMF oxidation and H₂ evolution catalyzed by Co—P/Co—P was shown in FIG. 79, which required only about 1.44 V to reach a current density of about 20 mA/cm² in about 1.0 M KOH with about 50 mM HMF. In contrast, in the absence of HMF, the Co—P/Co—P coupled was able to catalyze overall water splitting to produce H₂ and O₂, but required an about 150 mV higher potential (about 1.59 V) to arrive at about 20 mA/cm². The generated H₂ accompanying HMF oxidation was quantified by gas chromatography (GC). FIG. 80 plots the GC-measured H₂ quantification overlying with the calculated H₂ amount assuming that all the passed charge was used to produce H₂. The nearly overlap of these H₂ amounts demonstrated an about 100% Faradaic efficiency for H₂ evolution. Characterization of the cathode Co—P/CF post H₂ production revealed its similarity to the as-prepared sample. Taken together, these results demonstrate that Co—P/CF was competent for both HMF oxidation and H₂ evolution and the integration of these two reactions in one electrolyzer was more energetically efficient and kinetically favorable than overall water splitting

This example demonstrates that electrodeposited Co—P on copper foam was an excellent electrocatalyst for the oxidative upgrading of HMF to FDCA with about 90% yield in alkaline media. In addition, Co—P was able to catalyze H₂ evolution as the cathode reaction. By employing a Co—P/Co—P catalyst couple for the simultaneous production of FDCA and H₂, an integrated bio-refinery electrolyzer was able to reach about 20 mA/cm² at about 1.44 V, which was about 150 mV smaller than that of overall water splitting. HMF is only one of many attractive biomass intermediates. This strategy of integrating electrocatalytic biomass upgrading with H₂ evolution may help develop advanced catalysts for the production of a wide variety of valuable bio-products, boosting energy conversion efficiencies of electrolyzers and producing sustainable and non-fossil-based carbon-containing compounds.

### Example 5

### A General Strategy for Decoupled Hydrogen Protection from Water Splitting by Integrating Oxidative Biomass Valorization

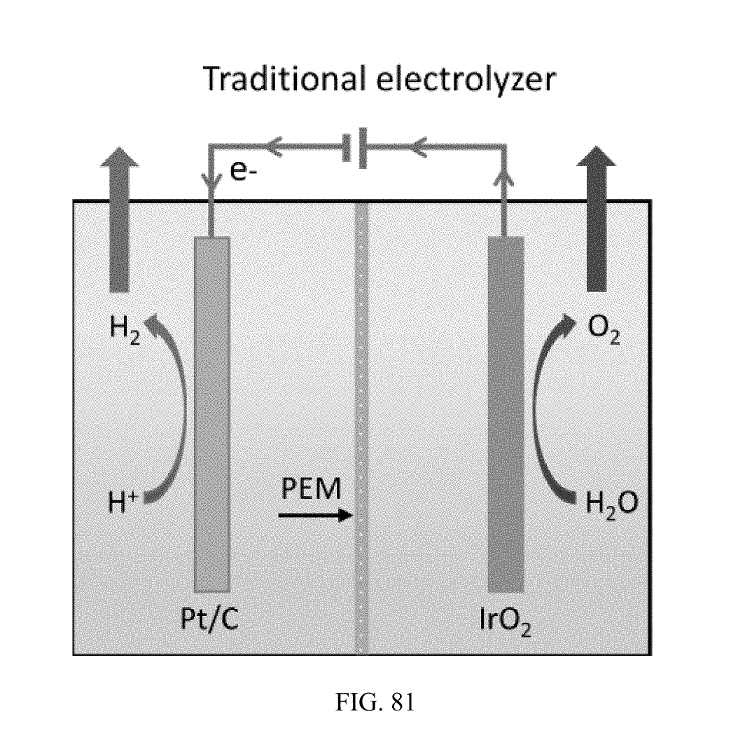

Room-temperature water electrolysis can be conducted under acidic conditions with PEM and catalyzed by Pt and IrO₂ (or RuO₂) catalysts for H₂ and O₂ evolution reactions (HER and OER), respectively (FIG. 81). However, during water electrolysis, H₂ and O₂ are produced simultaneously, which might lead to the formation of explosive H₂/O₂ mixture (gas crossover) even when a “gas impermeable” PEM is employed. Meanwhile, the coexistence of H₂, O₂, and water splitting catalysts may yield reactive oxygen species (ROS) that can degrade PEM and thus result in premature device failure, making PEM electrolyzer expensive for large-scale employment. Furthermore, the overall reaction rate is often limited by the anodic OER rate because of its more sluggish kinetics. Therefore, much higher overpotential is typically required for OER to match up the rate of HER, lowering the overall energy conversion efficiency.

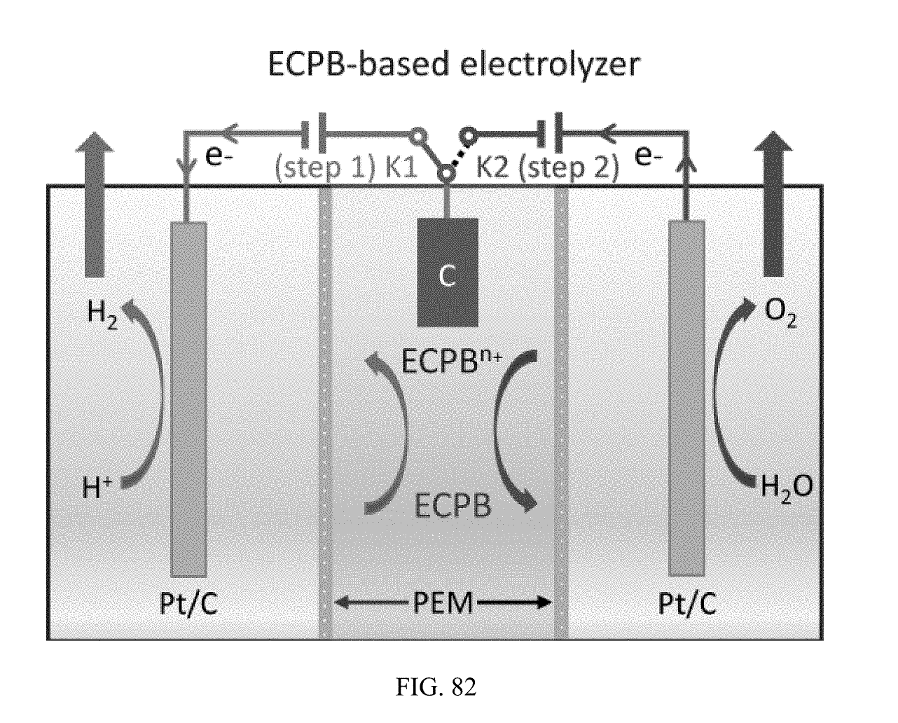

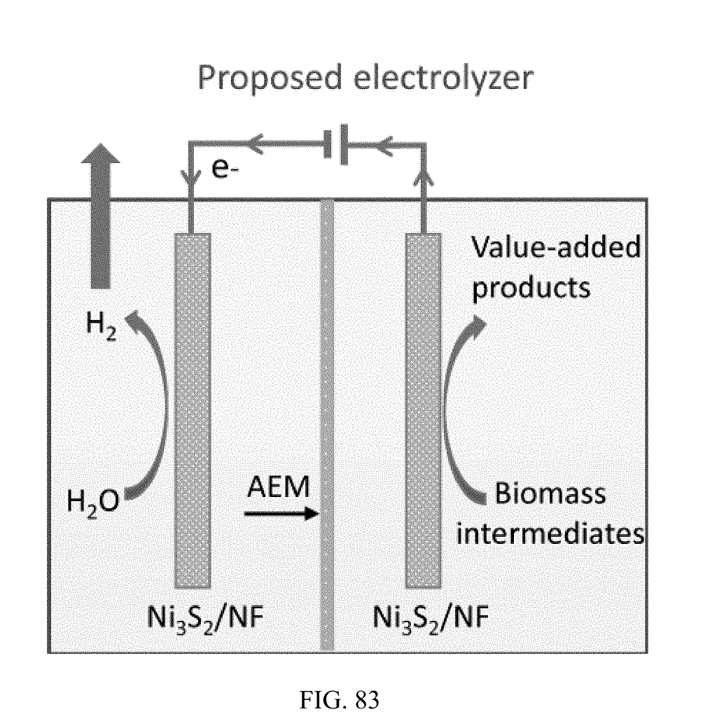

Electron-coupled proton buffers (ECPBs) may be utilized to split conventional PEM-based water electrolysis into two separate steps using redox mediators like silicotungstic acid, phosphomolybdate, and quinone derivatives (FIG. 82). Coupled with the appropriate redox processes of ECPBs, H₂ and O₂ can be generated in different time and space. For example, proton reduction occurs at a Pt cathode to produce H₂ while ECPB is oxidized to ECPBⁿ⁺ at a carbon electrode (FIG. 82 first step), wherein no O₂ is formed. Subsequently, water oxidation takes place at another Pt anode to produce O₂ and ECP13¹¹⁺is reduced back to the original ECPB on the same carbon electrode to complete the water electrolysis process (FIG. 82 second step). This strategy not only prevents gas mixing in the headspace of high-pressure water electrolyzers but also allows greater flexibility regarding membranes and electrodes. However, the requirement of double-membranes and three-compartment configuration complicates device construction and would increase the manufacture cost. Furthermore, it still requires high overpotential to catalyze OER, whose product, O₂, is not of high value. In addition, most earth-abundant OER electrocatalysts cannot survive the strongly acidic environment of the ECPB-containing electrolytes. As schematically shown in FIG. 83, a two-compartment configuration with an anion exchange membrane (AEM) is presented, wherein an alternative organic oxidation rather than OER occurs in the anodic compartment for the production of value-added and nongaseous products. With the appropriate selection of organic substrates and electrocatalysts, this strategy is able to reduce the voltage input for H₂ production, generate high-value products at anode, increase energy conversion efficiency, and circumvent H₂/O₂ mixing and ROS formation in traditional water electrolysis.

Oxidative biomass upgrading may be important in converting biomass-derived feedstocks to many value-added chemicals. Those oxygenated compounds can be primary building blocks to produce a diverse array of large-scale commodities, polymers, and pharmaceuticals. As disclosed in this example, five exemplary biomass intermediates, ethanol, benzyl alcohol, furfural, furfuryl alcohol, and 5-hydroxymethylfurfural (HMF), represented typical organic substrates for electrocatalytic oxidative upgrading integrated with decoupled H₂ production from water splitting. This strategy avoids the issues of H₂/O₂ mixing and ROS formation and produces valuable products at both electrodes with higher energy conversion efficiency than that of sole water splitting. Conversion of biomass into fuels and chemicals, generally called “biorefinery technology”, is an alternative to petroleum refining. For instance, HMF is the dehydration product of C6 carbohydrates and can act as a platform precursor for the synthesis of a wide variety of commodity fine chemicals, plastics, pharmaceuticals, and liquid fuels. For example, 2,5-furandicarboxylic acid (FDCA), one of the value-added products of HMF oxidation, can be used as an alternative monomer of terephthalic acid to produce polyamides, polyesters, and polyurethanes. Electrocatalytic oxidation represents a more sustainable alternative as the conversion can be driven by electricity and no chemical oxidants are needed. But the usage of expensive metal electrodes and redox mediator (e.g., TEMPO) would result in a high cost for the whole process.

In this example is demonstrated the electrocatalytic oxidation of five representative biomass compounds to value-added products catalyzed by hierarchically porous Ni₃S₂/Ni foam (Ni₃S₂/NF) under alkaline condition. Accompanying the biomass oxidation at the anode, H₂ production can take place at the cathode, which is also catalyzed by Ni₃S₂/NF due to its bifunctionality. In this scenario (FIG. 83), H₂ and value-added organic products are produced simultaneously and no O₂ is generated at the anode. Moreover, the Ni₃S₂/NF electrocatalyst can be prepared by a one-step sulfurization of commercial nickel foam. In certain embodiments, this strategy has at least four advantages: (1) the electrolyzer of integrated biomass oxidation and HER is able to deliver a large current density (e.g., about 100 mA cm-2) at a voltage about 200 mV smaller than that of pure water splitting electrolysis, hence increasing the energy conversion efficiency; (2) the oxidation products are more valuable than the starting organic substrates and significantly more useful than O₂, therefore maximizing the investment return; (3) organic acid oxidation products will stay in the electrolyte phase and no gas mixing or ROS issues will emerge; and (4) alkaline electrolyte makes it possible to employ many nonprecious electrocatalysts. This concept may be extendable to combine HER with many other organic oxidation reactions catalyzed by diverse inexpensive bifunctional electrocatalysts for multiple energy-related applications.

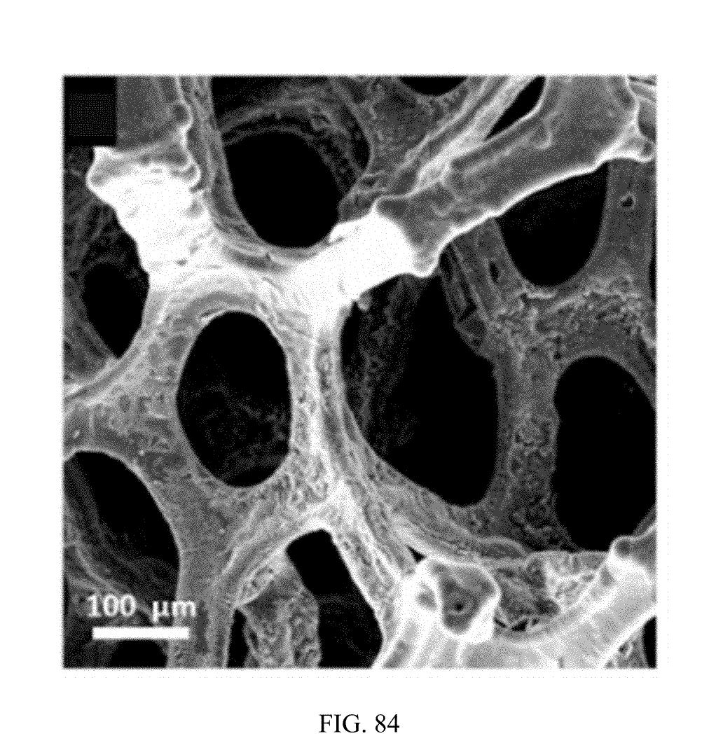

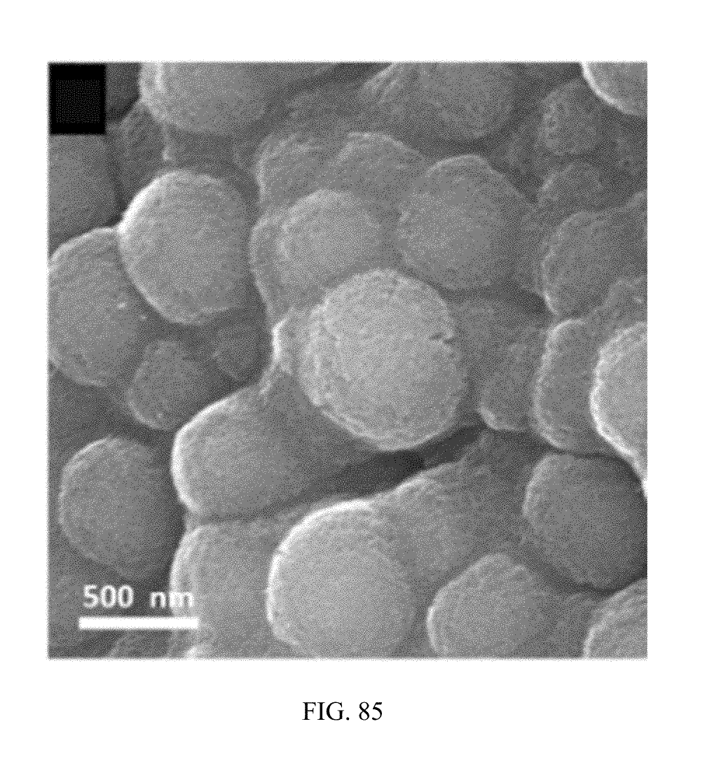

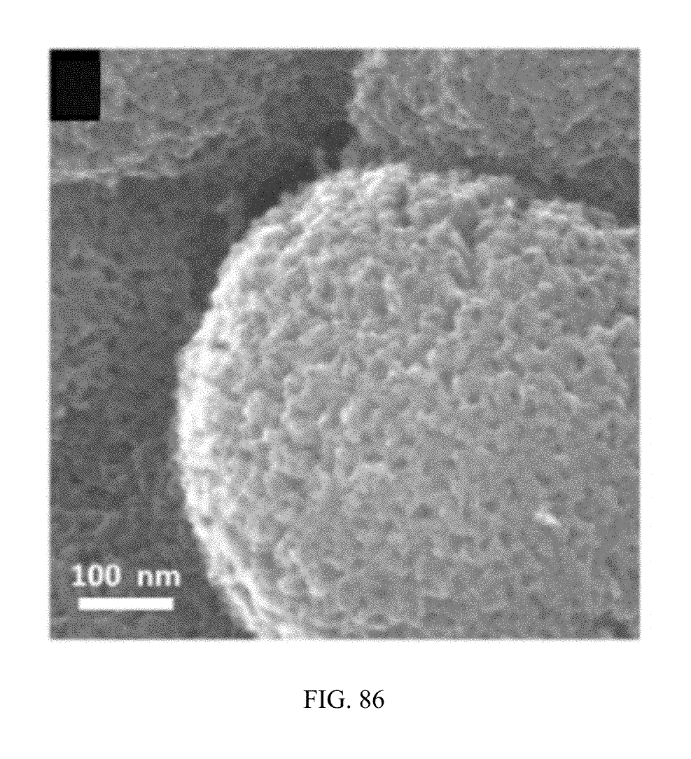

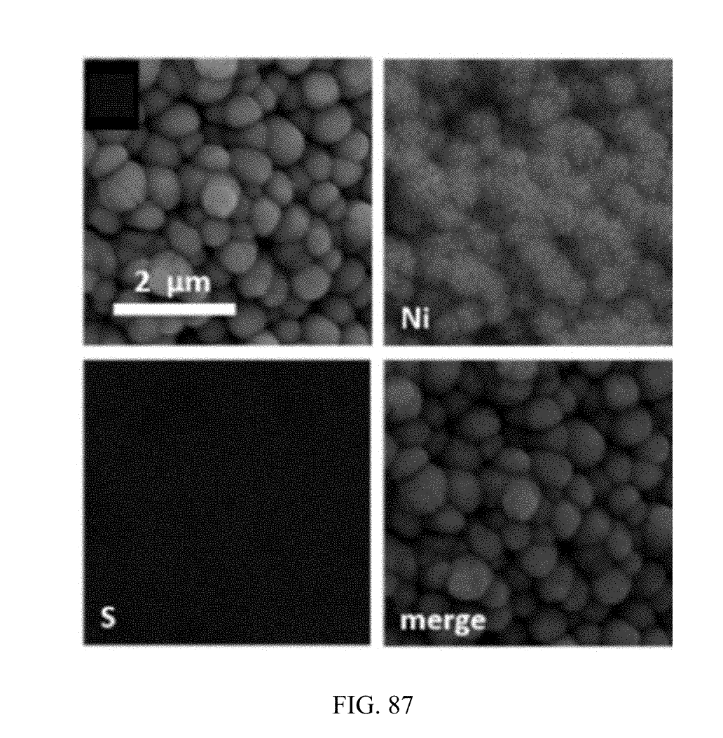

In order to develop bifunctional electrocatalysts for both anode and cathode reactions, Ni₃S₂/NF was chosen as a readily prepared and low-cost exemplary electrocatalyst. After sulfurization, the XRD pattern of the resulting foam confirmed the partial transformation of nickel foam to Ni₃S₂. Low-magnification SEM images of Ni₃S₂/NF indicated an interconnected, macroporous 3D framework (FIG. 84), similar to that of the pristine nickel foam. In sharp contrast to the featureless morphology of nickel foam, high-magnified SEM images of Ni₃S₂/NF revealed an interesting structure composed of stacked nanoparticles (FIG. 85). A closer inspection of these nanoparticles in a high-resolution SEM image exhibited the presence of numerous mesopores on the surface of Ni₃S₂/NF (FIG. 86). Such a unique hierarchically porous nanoarchitecture may facilitate the accessibility of catalytically active sites and benefit mass transport, thereby promoting the electrochemical activity. Elemental mapping analysis of Ni₃S₂/NF showed the uniform distribution of Ni and S (FIG. 87), corroborating the successful chemical conversion of Ni into Ni₃S₂. In line with the elemental mapping results, XPS analysis further verified the presence of Ni and S in Ni₃S₂/NF. High-resolution Ni 2₁)_{3/2} spectrum was deconvoluted into three sub-peaks at binding energies of about 852.9, about 855.8, and about 861.0 eV, assignable to Ni^{δ+} in Ni₃S₂, oxidized Ni species, and Ni 2₁)_{3/2} satellite peak, respectively. Similarly, the high-resolution S 2p XPS spectrum could be fitted by three sub-peaks at about 161.9, about 162.8, and about 165.6 eV, corresponding to S 2p_{3/2}, S 2p_{1/2}, and oxidized sulfur species, respectively. Collectively, all of these results support the successful formation of Ni₃S₂/NF. The oxidized Ni and S species in Ni₃S₂/NF could be ascribed to superficial oxidation due to air contact.

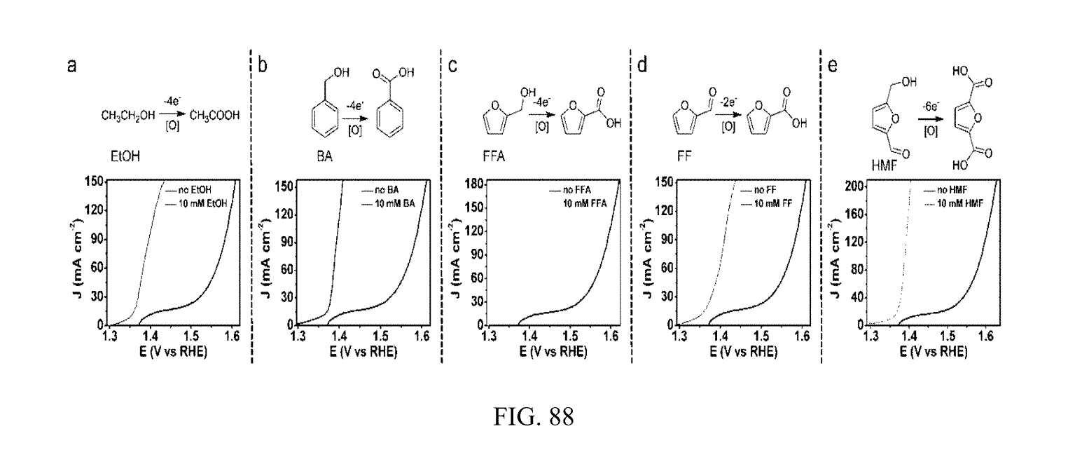

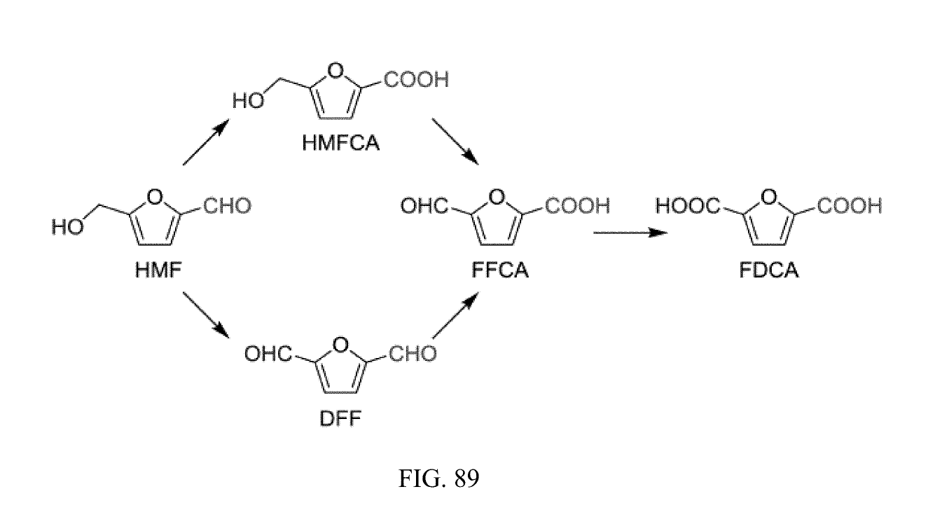

The electrocatalytic oxidation of the five representative biomass substrates, ethanol (EtOH), benzyl alcohol (BA), furfural (FF), furfuryl alcohol (FFA), and HMF, were then investigated using Ni₃S₂/NF as the electrocatalyst in about 1.0 M KOH. For comparison purposes, water oxidation in the absence of any organic compounds was also conducted with Ni₃S₂/NF under the same conditions. An initial electrochemical activation phenomenon was observed for Ni₃S₂/NF under anodic treatment. Therefore, all the following LSV curves in this example were collected after the cessation of each catalyst activation in pure about 1.0 M KOH without organic substrates. As shown in FIG. 88, Ni₃S₂/NF exhibited an onset potential of about 1.50 V vs RHE and high catalytic current density beyond about 1.55 V vs RHE for OER, implying its excellent water oxidation activity. After introducing about 10 mM biomass substrates, the onset potentials all shifted to about 1.35 V vs RHE and rapid current density rises were observed within about 1.40 V vs RHE, indicative of more favorable biomass oxidation than OER. The corresponding oxidation reactions were shown on top of the LSV curves. To achieve benchmark current densities of about 50, about 100, and about 150 mA cm⁻², the overpotentials for these biomass oxidation reactions were at least about 160 mV smaller than that of OER (FIG. 88), highlighting the better energy conversion efficiency. Remarkably, the required potential to afford about 100 mA cm⁻² for HNIF oxidation was significantly reduced by about 200 mV. Chronoamperometry experiments carried out at about 1.423 V for the oxidation of the five organic substrates demonstrated almost complete conversion to their corresponding high-value products after passing the theoretical amount of charge, as revealed by the HPLC and ¹H NMR results (FIGS. 89-92). Nearly unity Faradaic efficiencies were achieved for those biomass oxidations. The pristine nickel foam showed much inferior performance for both OER and biomass oxidation, perhaps highlighting the importance of sulfurization.

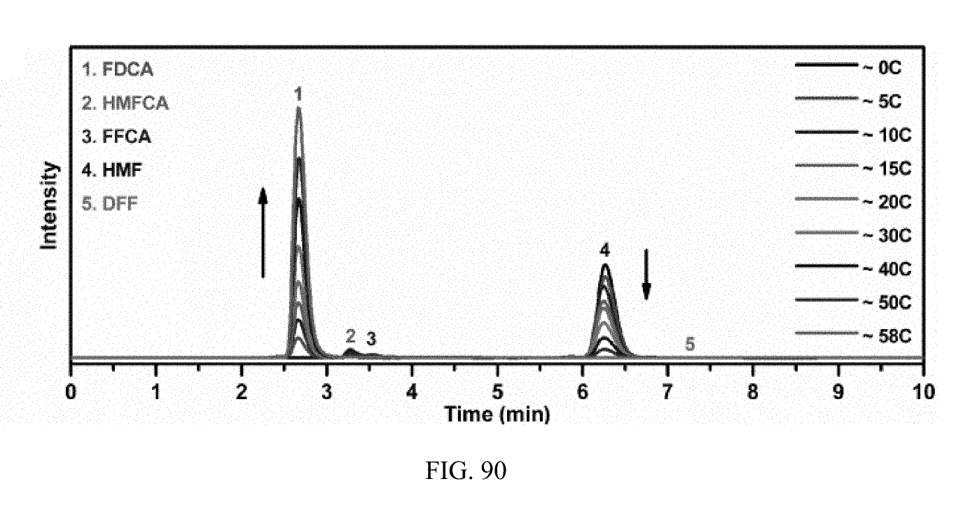

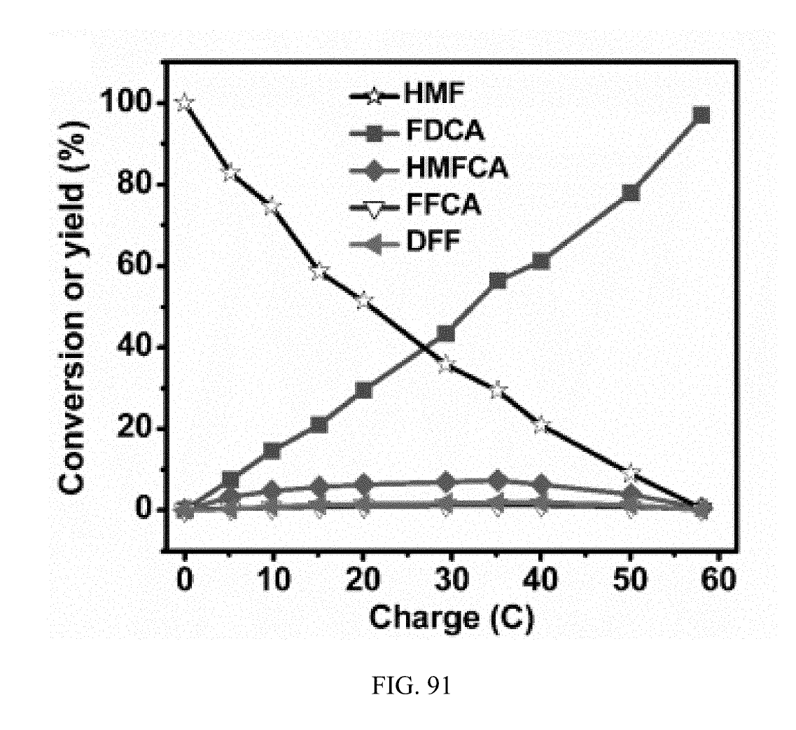

In order to gain more insights into the electrocatalytic oxidation of these organic substrates, HMF oxidation was chosen as an exemplary case study. Generally, aerobic oxidation of HMF follows two pathways. One is through an initial alcohol oxidation to form DFF as the intermediate (FIG. 89); while the other is from an initial aldehyde oxidation to yield HMFCA first (FIG. 89). Both pathways converge at the formation of FFCA prior to FDCA. In order to identify and quantify the oxidation intermediates and final product of HMF oxidation as well as calculate the corresponding Faradaic efficiencies, Ni ₃S₂/NF-catalyzed HMF oxidation was conducted in about 10 mL about 1.0 M KOH with about 10 mM HMF at an applied potential of about 1.423 V vs RHE. About 58 C charge was calculated to convert all of the HMF to FDCA if a 100% Faradaic efficiency could be achieved. In fact, as implied in FIG. 88e, no appreciable water oxidation could occur at about 1.423 V, and hence a high Faradaic efficiency for HMF oxidation was likely. FIG. 90 showed the HPLC chromatograms of HMF and its oxidation intermediates (HMFCA, DFF, and FFCA) and product (FDCA) during electrolysis. It exhibits the decrease of HMF and rise of FDCA over time, suggesting the conversion of HMF to FDCA. After passing charge of about 58 C, the HPLC trace of HMF disappeared while that of FDCA rose to the maximum, which signified the complete conversion of HMF. The conversion of HMF and the yields of its oxidation intermediates as well as the final product FDCA during the electrolysis were plotted in FIG. 91, resulting in Faradaic efficiencies of about 100% and about 98% for HMF conversion and FDCA production, respectively.

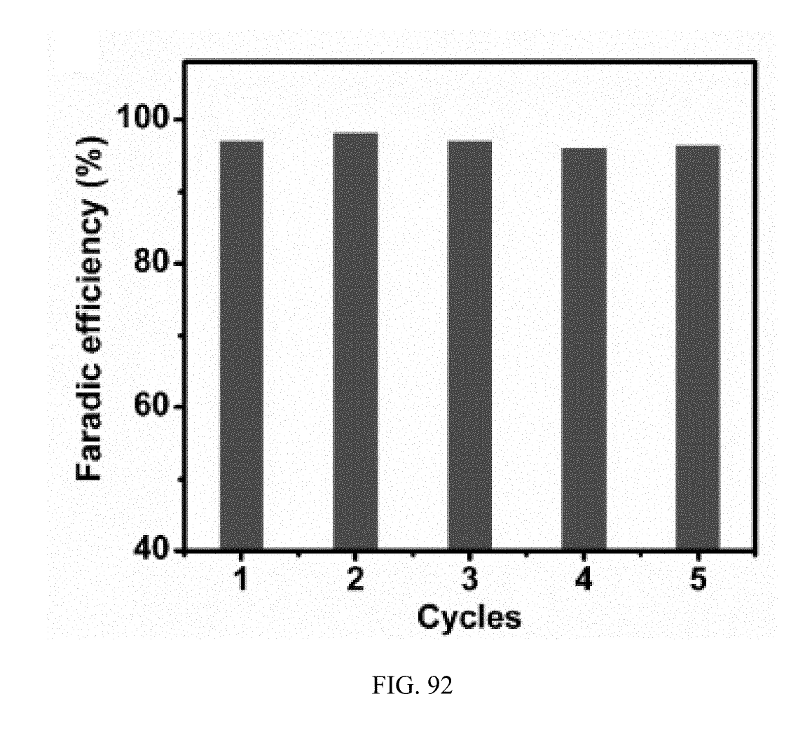

Five successive cycles of the above constant potential electrolysis utilizing the same Ni₃S₂/NF were performed to evaluate its durability toward HMF oxidation. As shown in FIG. 92, the calculated Faradaic efficiencies for FDCA formation were in the range of about 96-99%, illustrating the robust stability of Ni₃S₂/NF for HMF oxidation.

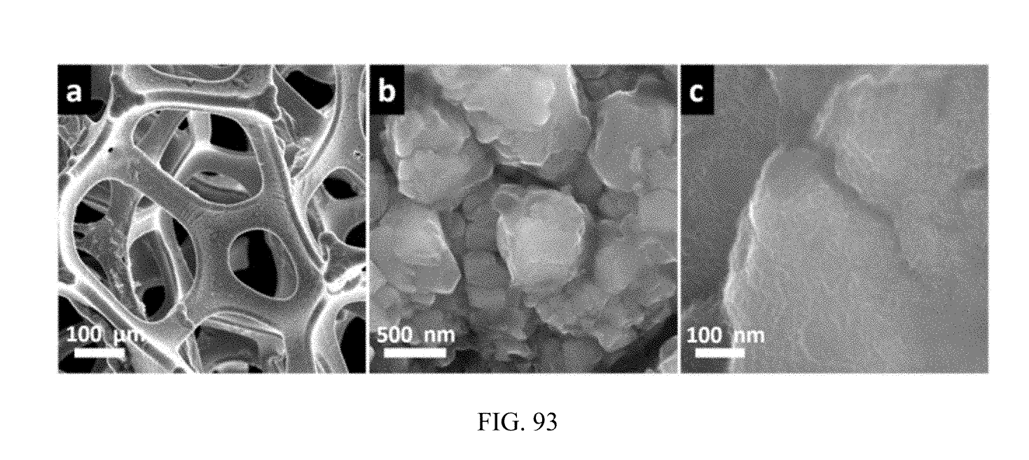

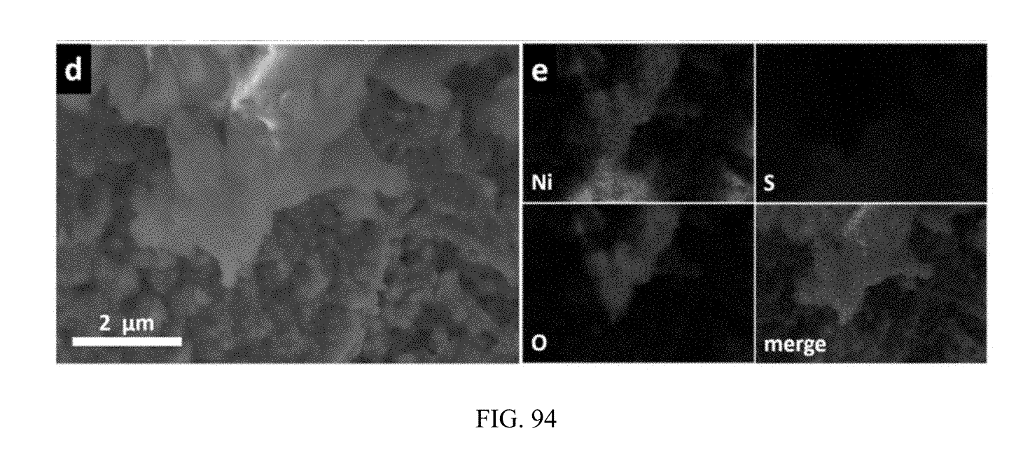

XRD, SEM, and XPS were employed to interrogate the structure and composition details of the Ni₃S₂/NF electrocatalyst after the stability testing (named as post-HMF Ni₃S₂/NF). Although the low-magnified SEM image (FIG. 93a) and XRD pattern of post-HMF Ni₃S₂/NF indicated the maintenance of the overall 3D hierarchically porous configuration and primary Ni₃S₂ phase, respectively, a close inspection of its high-magnified SEM images (FIG. 93b-c and FIG. 94d) revealed the presence of featureless monoliths, different from the fresh sample (FIG. 86). Elemental mapping images (FIG. 94e) indicated that the post-HMF Ni₃S₂/NF mainly consisted of Ni and S, plus a large concentration of 0 over the newly formed monoliths. On the other hand, the high-resolution Ni 2p XPS spectrum of the post-HMF Ni₃S₂/NF demonstrated the disappearance of the peak at about 852.9 eV (assignable to Ni^{δ+} in Ni₃S₂) and an increase of the peak at about 855.8 eV (corresponding to oxidized Ni species), suggesting the oxidation of Ni₃ S₂. This oxidation phenomenon was also supported by the increased intensity of the peak ascribed to oxidized S species in the high-resolution S 2p XPS spectrum. Collectively, the real catalytic active sites of Ni₃S₂/NF for HMF oxidation reaction might be attributed to oxidized Ni species (nickel oxides/hydroxides and oxyhydroxides), which might also be the true active species in bifunctional electrocatalysts of overall water splitting for OER.

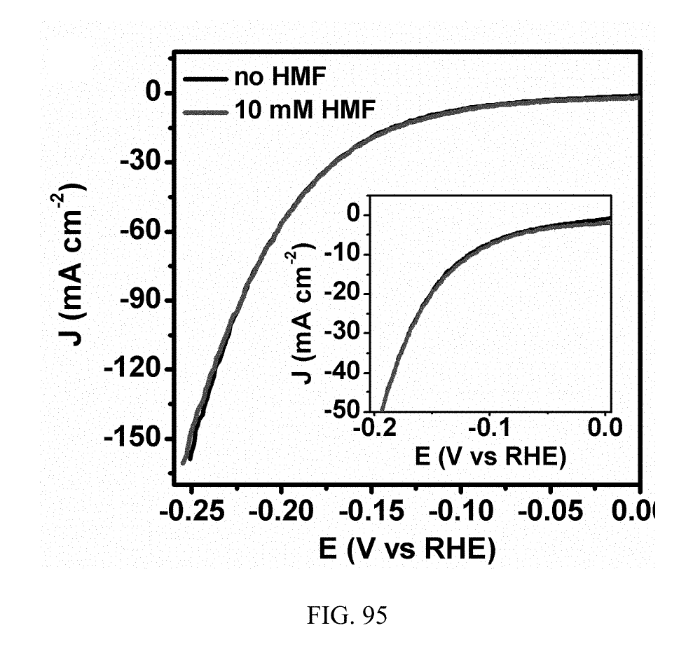

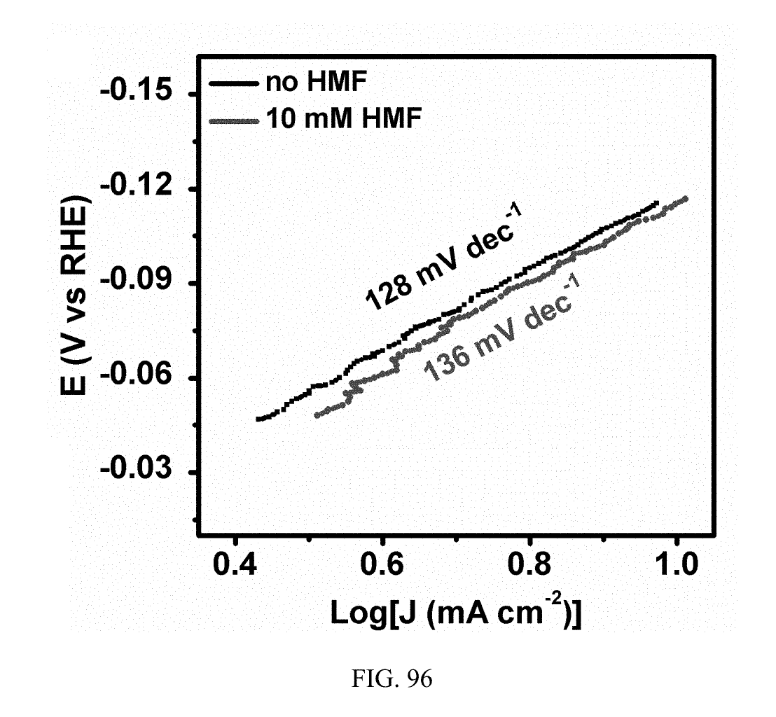

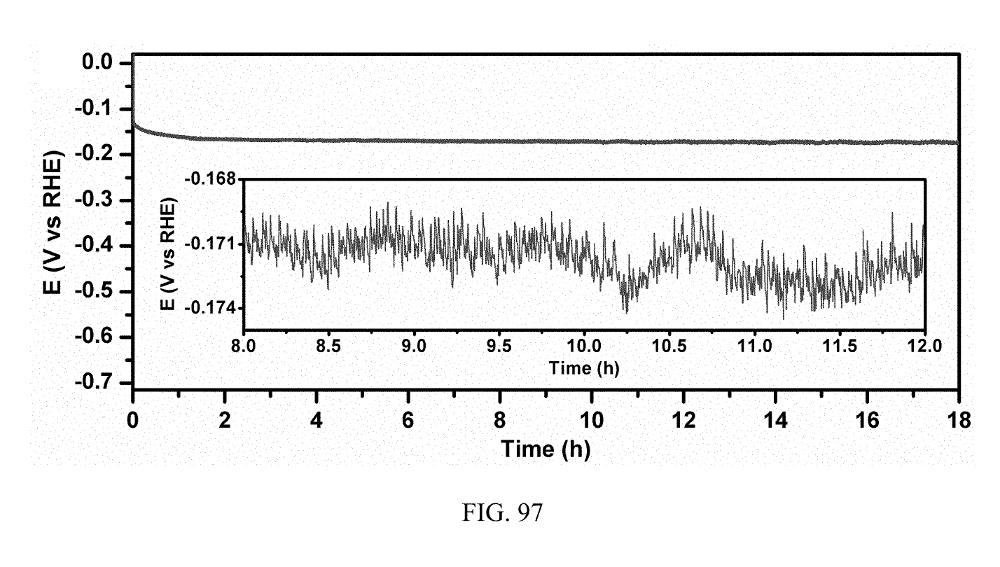

Even though a two-compartment configuration with an anion exchange membrane (AEM) was employed, there still exists a certain possibility that organic species in the anode compartment would penetrate through the membrane and migrate into the cathode compartment. In order to successfully couple HER and biomass oxidation with maximum Faradaic efficiency, the electrocatalyst at the cathode should exhibit high preference for HER and strong tolerance to the presence of the selected biomass intermediates. Therefore, the impact of HMF on the HER activity of Ni₃S₂/NF under the harshest condition was evaluated (assuming all the HMF was present in the cathode compartment). As demonstrated in FIG. 95, the HER polarization curves of Ni₃S₂/NF in about 1.0 M KOH in the absence and presence of about 10 mM HMF almost overlapped and the calculated Tafel slope only increased from about 128 to about 136 mV dec⁻¹ (FIG. 96). In addition, an about 18 h chronopotentiometry experiment conducted at a current density of about −10 mA cm⁻² in about 1.0 M KOH with about 10 mM HMF showed that the required potential increased by less than about 40 mV (FIG. 97). The fluctuation of an expanded chronopotentiometric curve also implied the formation and release of H₂ bubbles on the catalyst surface (FIG. 97 inset). The Ni₃S₂/NF required an overpotential of only about −116 mV to reach about 10 mA cm⁻², which is lower than those of nonprecious HER electrocatalysts including MoCₓ/C (about −151 mV), high-index faceted Ni₃ S₂ nanosheets arrays (about −223 mV), and NiFe LDH/NF (about −200 mV), implying its excellent electrocatalytic HER activity. Similar resistance of its HER performance to the other four organic compounds was also confirmed from the analogous HER electrolysis conducted in the presence of those organic compounds.

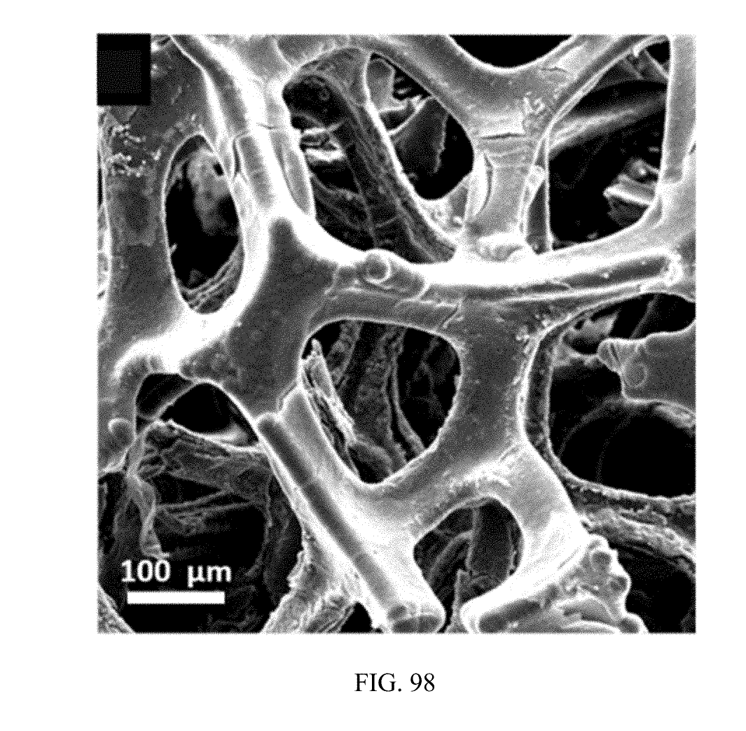

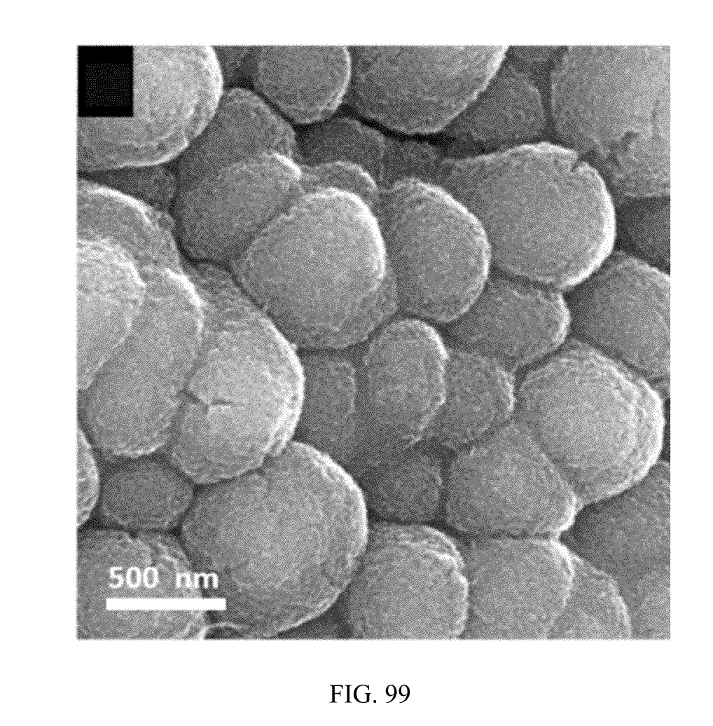

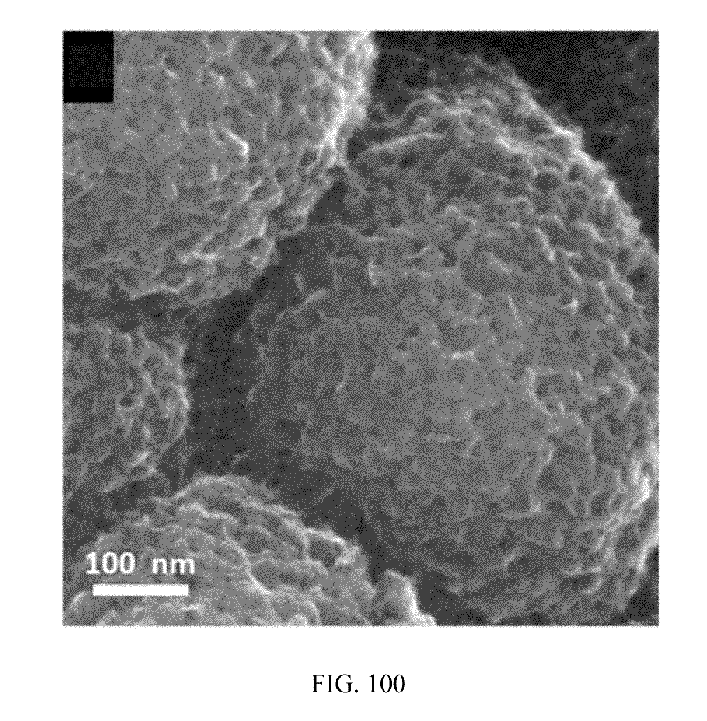

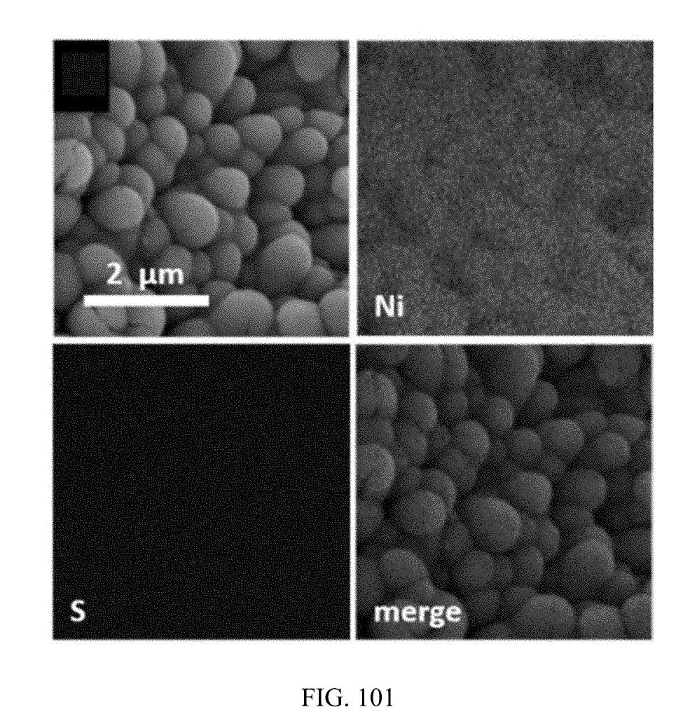

Post HER electrolysis characterization was also conducted to probe the morphology and composition details of Ni₃S₂/NF after the about 18 h HER stability test in about 1.0 M KOH with about 10 mM HMF (denoted as post-HER with HMF Ni₃S₂/NF). The low-magnification SEM image in FIG. 98 demonstrated that the post-HER with HMF Ni₃S₂/NF still maintained the overall 3D hierarchically porous structure. High-magnification SEM images (FIGS. 99 and 100) suggested that its morphology of porous nanoparticles was similar to that of the fresh Ni₃S₂/NF (FIG. 86). The corresponding elemental mapping results indicated the retained uniform spatial distribution of Ni and S in the post-HER with HMF Ni₃S₂/NF (FIG. 101). The XRD pattern demonstrated the presence of Ni₃S₂ composition, nearly identical to that of the as-prepared catalyst. In addition, the similarity of high-resolution Ni and S XPS spectra of the fresh and post-HER with HMF Ni₃S₂/NF samples also confirmed the retention of the electrocatalysts in terms of composition and oxidation state, corroborating its superior robustness for HER electrocatalysis and excellent tolerance towards HMF.

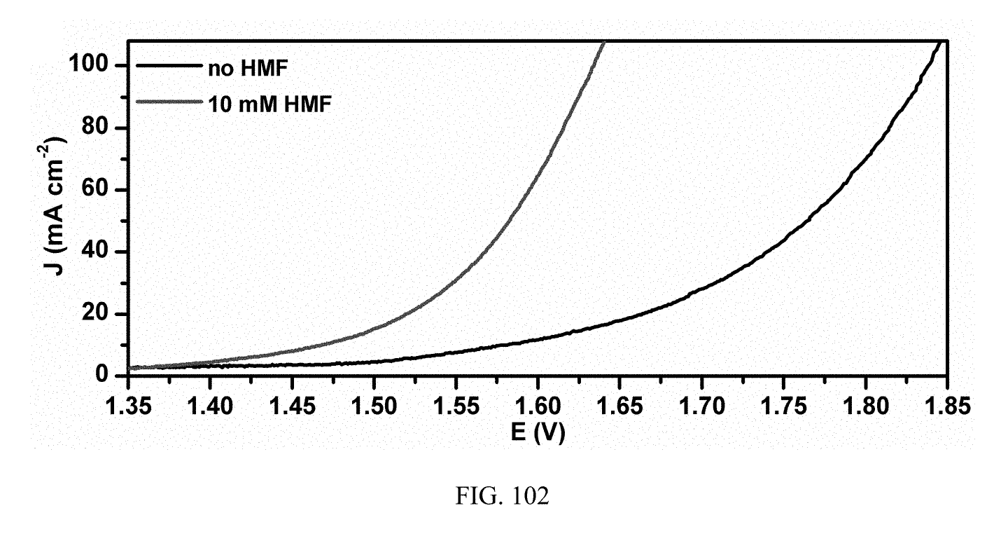

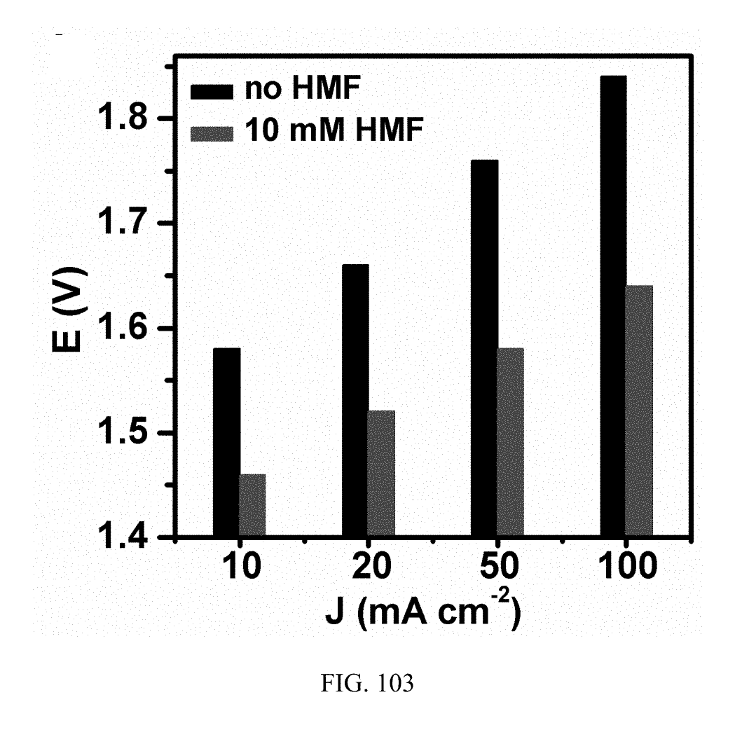

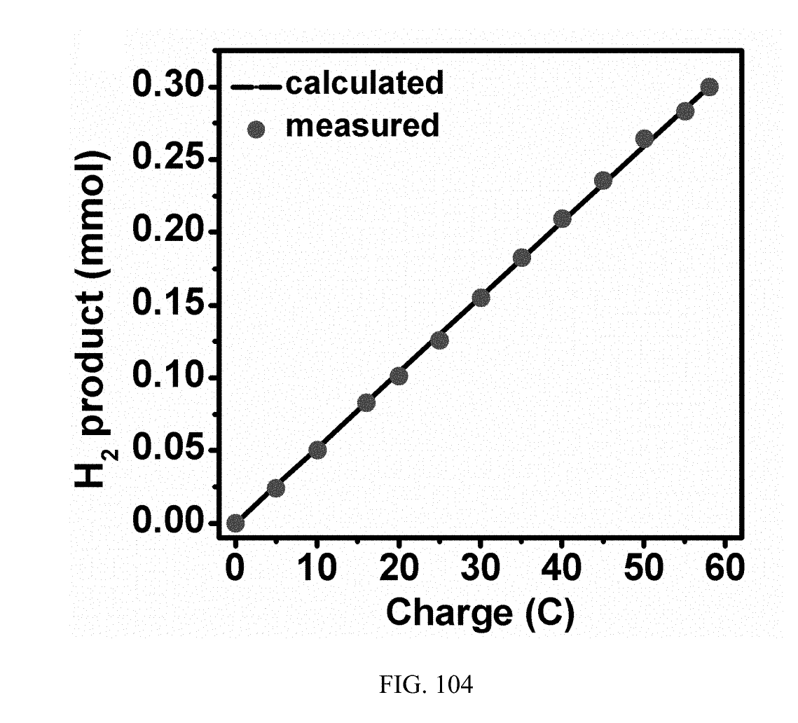

Collectively, all the aforementioned results demonstrated that the Ni₃S₂/NF is able to catalyze the oxidation of biomass intermediates and H₂ evolution under alkaline condition simultaneously. Hence, a two-electrode electrolyzer employing a Ni₃S₂/NF electrocatalyst couple for both anode and cathode was constructed. As shown in FIG. 102, the LSV curve of the Ni₃S₂/NF catalyst couple in about 1.0 M KOH displayed an onset around about 1.55 V for overall water splitting in the absence of HMF. In order to achieve a catalytic current density of about 10, about 20, about 50, and about 100 mV cm⁻², cell voltages of about 1.58, about 1.66, about 1.76, and about 1.84 V, respectively, may be required. In fact, these voltages are smaller than those of many bifunctional electrocatalysts for overall water splitting, such as Co—P (about 1.64 V for about 10 mA cm⁻²), NiFe LDH/NF (about 1.70 V for about 10 mA cm⁻²), CoOₓ@CN (about 1.90 V for about 50 mA cm⁻²), and Ni₅P₄/NF (about 1.70 V for about 10 mA cm⁻²). Upon the addition of about 10 mM HMF, the catalytic onset potential was reduced to about 1.40 V and the cell voltages were further lowered to about 1.46, about 1.52, about 1.58, and about 1.64 V to achieve about 10, about 20, about 50, and about 100 mA cm⁻², respectively (FIG. 103), implying much better energy conversion efficiency of Ni₃S₂/NF-catal yzed HER and HMF oxidation relative to water splitting (save about 200 mV to deliver about 100 mV cm⁻²). To quantify the produced H₂ and FDCA under this two-electrode configuration, a long-term electrolysis at a constant cell voltage of about 1.50 V vs RHE was executed to pass the charge of about 58 C. As shown in FIG. 104, the generated H₂ quantified by gas chromatography (GC) matched the theoretically calculated amount very well. Analysis of the resulting electrolyte by HPLC also resulted in an about 98% Faradaic efficiency for the FDCA production.

In this example is demonstrated a general strategy for decoupled H₂ generation from water splitting by combining oxidative biomass upgrading to value-added products with a low-cost and hierarchically porous Ni₃S₂/NF bifunctional electrocatalyst. In the current exemplary case, H₂ evolution occurs at the cathode catalyzed by Ni₃S₂/NF; while simultaneously oxidative upgrading of biomass intermediates is catalyzed by the Ni₃S₂/NF-derived catalyst at the anode to more valuable bioproducts. Both reactions take place with Faradaic efficiencies close to 100%. Owing to the more favorable thermodynamics of these biomass oxidations than that of OER, the cell voltage to reach benchmark current densities (e.g., about 100 mA cm⁻²) for H₂ production is reduced by about 200 mV relative to that of sole water splitting. Additionally, more valuable bioproducts (rather than O₂) are generated at the anode. Because of no O₂ production, such a new type electrolyzer could circumvent the potential H₂/O₂ mixing and ROS formation, beneficial to the long lifespan of an electrolyzer and reduce the maintenance cost. In addition, the alkaline electrolyte enables the possibility of employing nonprecious electrocatalysts. All of the above advantages render this integrating strategy very appealing to combine HER with many other organic oxidation reactions for multiple energy-related applications. Despite the different thermodynamic changes of the oxidation reactions of those five biomass intermediates to their corresponding products, the electrocatalytic currents took off at very similar potentials (about 1.35 V vs RHE, FIG. 88), which may indicate that the overpotential requirement was largely determined by the oxidation of the electrocatalyst to its functional oxidation state.

Chemicals: 5-hydroxymethylfurfural (HMF), furfuryl alcohol (FFA), potassium hydroxide (KOH), and sulfur were purchased commercially. 2,5-Furandicarboxylic acid (FDCA) was purchased commercially. 2,5-Diformylfuran (DFF) and 2-formyl-5-furancarboxylic acid (FFCA) were purchased commercially. 5-hydroxymethyl-2-furan-carboxylic acid (HMFCA) was purchased commercially. Ethanol (EtOH) was purchased commercially. Benzyl alcohol (BA) and furfural (FF) were purchased commercially. Nickel foam with purity greater than about 99.99% was purchased commercially. All chemicals in this example were used as received without any further purification. Deionized water (about 18 MΩ·cm) from a Barnstead E-Pure system was used in all experiments in this example.

Synthesis of Ni₃S₂/NF: In a typical preparation, a piece of nickel foam (NF) with the size of about 0.5 cm×about 0.5 cm was placed at the center of a tube furnace and about 0.5 g sulfur was placed at the upstream side of the furnace at a carefully adjusted location. After flushing with Ar for about 15 min, the temperature of the furnace was quickly elevated to the reaction temperature of about 280° C. with a ramping rate of about 10° C. min⁻¹ and kept at about 280° C. for about 10 min to partially convert the metallic nickel to nickel sulfides. After cooling down to room temperature, the desired Ni₃S₂/NF was obtained.

Physical Methods: XPS samples were affixed on a stainless steel Kratos sample bar, loaded into an instrument's load lock chamber, and evacuated to about 5×10⁻⁸ torr before transferred into a sample analysis chamber under ultrahigh vacuum condition (about 10⁻¹⁰ torr). XPS spectra were collected using the monochromatic Al K_{α} source (about 1486.7 eV) at an about 300 μm×about 700 μm spot size. Low-resolution survey and high-resolution region scans at the binding energy of interest were collected for each sample. To minimize charging, all samples were flooded with low-energy electrons and ions from the instrument's built-in charge neutralizer. The samples were also sputter cleaned inside the analysis chamber with about 1 keV Ar⁺ ions for about 30 seconds to remove adventitious contaminants and surface oxides. XPS data were analyzed using CasaXPS and the energy correction on high-resolution scans was calibrated by referencing the C is peak of adventitious carbon to about 284.5 eV.

Electrocatalytic Experiments: Electrochemical HER, OER, and biomass oxidation measurements were performed with a three-electrode configuration. The as-prepared Ni₃S₂/NF was directly used as the working electrode, a Ag/AgCl (sat. KCl) electrode as the reference electrode, and a carbon rod as the counter electrode. All the potentials reported in this example were quoted with respect to the reversible hydrogen electrode (RHE) through RHE calibration. The calibration was performed in high-purity H₂ saturated electrolyte (about 1.0 M KOH) with a Pt wire as the working electrode. Cyclic voltammetry (CV) was conducted at a scan rate of about 1 mV s¹ and the average of the two potentials at which the current crossed zero was taken to be the thermodynamic potential for the hydrogen electrode reaction. The electrochemical HER, OER, and biomass oxidation experiments were conducted in about 10 mL about 1.0 M KOH solution with and without organic substrates. For two-electrode electrolysis, Ni₃ S₂/NF was employed as the catalyst for both anode and cathode. The linear sweep voltammetry (LSV) with the two-electrode configuration was scanned at a scan rate of about 2 mV s⁻¹. iR (current times internal resistance) compensation was applied in all the electrochemical experiments in this example to account for the voltage drop between the reference and working electrodes. The stability test of Ni₃S₂/NF for biomass oxidation was evaluated by chronoamperometry at about 1.423 V vs RHE in about 10 mL about 1.0 M KOH with about 10 mM organic substrates for five successive trials.

Product Quantification: To analyze the products of HMF oxidation quantitatively and calculate the corresponding Faradaic efficiencies, about 10 νL of the electrolyte solution during chronoamperometry at about 1.423 V vs RHE (for three-electrode configuration) or at the cell voltage of about 1.5 V (for two-electrode configuration) was withdrawn from the electrolyte solution and diluted with about 490 νL water, which was then analyzed using high-performance liquid chromatography (HPLC) at about room temperature. The HPLC was equipped with an ultraviolet-visible detector set at about 265 nm and an about 4.6 mm×about 150 mm Shim-pack GWS 5 μm C 18 column. A mixture of eluting solvents (A and B) was utilized. Solvent A was about 5 mM ammonium formate aqueous solution and solvent B was methanol. Separation and quantification were accomplished using an isocratic elution of about 70% A and about 30% B for about 10 min run time and the flow rate was set at about 0.5 mL mid'. The identification and quantification of the products were determined from the calibration curves by applying standard solutions with known concentrations of commercially purchased pure reactants, intermediates, and final products. The ¹H NMR spectra were collected on a 500 MHz NMR.

The conversion (%) of organic substrates and the yield (%) of oxidation products were calculated based on the following two equations:

$$Conversion ( \% ) = \frac{mole of substrate consumed}{mole of initial substrate} \times 100 \%$$

$$Yield ( \% ) = \frac{mole of product formed}{mole of initial substrate} \times 100 \%$$

The Faradaic efficiency (FE) of product formation was calculated using the following equation:

$$FE ( \% ) = \frac{mole of product formed}{total charge passed / ( n \times F )} \times 100 \%$$

where n is the number of electron transfer for each product formation and F is the Faraday constant (about 96,485 C mol⁻¹).

What is claimed is:

1. A system for producing hydrogen gas and upgrading a biomass reactant, the system comprising:
an anode compartment comprising
an anode, and
an anode solution comprising water, a first electrolyte, and an alcohol or an aldehyde derived from a lignocellulosic biomass; and
a cathode compartment comprising
a cathode, and
a cathode solution comprising water and a second electrolyte;
wherein the anode and the cathode each independently comprise a catalyst loaded onto a conductive substrate, the catalyst having a metal component and a non-metal component, wherein the metal component is selected from the group consisting of cobalt, nickel, iron, copper, manganese and a combination thereof; and the non-metal component is selected from the group consisting of phosphorous, sulfur, nitrogen, oxygen and a combination thereof.

2. The system of claim 1, wherein the catalyst is selected from the group consisting of cobalt phosphide, nickel phosphide, cobalt sulfide, nickel sulfide, nickel nitride, cobalt oxide, nickel oxide, and a combination thereof.

3. The system of claim 1, wherein the conductive substrate is selected from the group consisting of copper, nickel, stainless steel, glassy carbon, nickel foam, stainless steel foam, titanium, fluorine-doped tin oxide, indium-doped tin oxide, and a combination thereof.

4. The system of claim 1, wherein the cathode and the anode each include the same catalyst and conductive substrate.

5. The system of claim 1, wherein the first and second electrolyte each independently comprise potassium hydroxide, sodium hydroxide, sodium perchlorate, borate buffer, phosphate buffer, or a combination thereof.

6. The system of claim 1, wherein the first and second electrolyte are each independently present at a concentration of from about 0.1 M to about 5 M.

7. The system of claim 1, wherein the alcohol or the aldehyde derived from a lignocellulosic biomass is present at a concentration of from about 1 mM to about 100 mM.

8. The system of claim 1, wherein the alcohol or the aldehyde derived from a lignocellulosic biomass is selected from the group consisting of 5-hydroxymethylfurfural (HMF), 3-hydroxypropionic acid, glycerol, sorbitol, xylitol, lactic acid, ethanol, butanol, benzyl alcohol, furfural, arabinitol, xylose, methanol, cinnamaldehyde, and a combination thereof.

9. The system of claim 8, wherein the alcohol or the aldehyde derived from a lignocellulosic biomass is selected from the group consisting of HMF, ethanol, benzyl alcohol, furfural and a combination thereof.

10. The system of claim 1, further comprising a reference electrode.

11. The system of claim 1, further comprising a separator between the anode compartment and the cathode compartment.

12. A method for producing hydrogen gas and upgrading a biomass reactant, the method comprising:
applying a voltage to the cathode and the anode of the system of claim 1,
whereupon applying the voltage, the alcohol or the aldehyde derived from a lignocellulosic biomass is oxidized in the anode compartment to form an aldehyde or a carboxylic acid biomass product and H⁺is reduced in the cathode compartment to provide hydrogen gas.

13. The method of claim 12, wherein the applied voltage is less than or equal to 2 V.

14. The method of claim 12, wherein the applied voltage provides a current density of from about 10 mA/cm² to about 100 mA/cm².

15. The method of claim 12, wherein the Faradaic efficiency of providing the hydrogen gas is greater than or equal to 95%.

16. The method of claim 12, wherein the Faradaic efficiency of providing the aldehyde or the carboxylic acid biomass product is greater than or equal to 95%.

17. The method of claim 12, wherein the anode and cathode solution each independently have a pH of from about 5 to about 9.

18. The method of claim 12, wherein the anode and cathode solution each independently have a temperature of from about 15° C. to about 30° C.

19. The method of claim 12, wherein the alcohol or the aldehyde derived from a lignocellulosic biomass and the aldehyde or the carboxylic acid biomass product are selected from one of the following:
the alcohol or the aldehyde derived from a lignocellulosic biomass is HMF and the aldehyde or the carboxylic acid biomass product is 2,5-furandicarboxylic acid (FDCA);
the alcohol or the aldehyde derived from a lignocellulosic biomass is ethanol and the aldehyde or the carboxylic acid biomass product is acetic acid;
the alcohol or the aldehyde derived from a lignocellulosic biomass is benzyl alcohol and the aldehyde or the carboxylic acid biomass product is benzoic acid;
the alcohol or the aldehyde derived from a lignocellulosic biomass is furfural and the aldehyde or the carboxylic acid biomass product is furoic acid; and
a combination thereof.

20. The method of claim 12, further comprising separating the hydrogen gas from the cathode compartment and the aldehyde or the carboxylic acid biomass product from the anode compartment.
